SEQUENCE LISTING (3 sequences)

SEQ ID NO: 1 - DNA, 43 bp, Homo sapiens
  source 1..43: mol_type = genomic DNA
ttttatatattygygcgtatgtggcgtatgtttttatgtggta

SEQ ID NO: 2 - DNA, 42 bp, Homo sapiens
  source 1..42: mol_type = genomic DNA
tcgggtagaagtgttttaaattattatgtatttaagaaatgt

SEQ ID NO: 3 - DNA, 43 bp, Homo sapiens
  source 1..43: mol_type = genomic DNA
gaaggggtttcgtcgttagtcgttagaggtygagtggtagggt

(19) United States
(12) Patent Application Publication
(10) Pub. No.: US 2026/0253674 A1
(43) Pub. Date: Aug. 27, 2026

(54) METHODS, SYSTEMS AND ASSOCIATED COMPUTER PROGRAM PRODUCTS FOR DISCRIMINATING TYPE OF BIOLOGICAL SAMPLE OF AN ORGANISM USING EPIGENETIC MODIFICATION INFORMATION

(71) Applicant: POMORSKI UNIWERSYTET MEDYCZNY W SZCZECINIE, Szczecin (PL)

(72) Inventors: Tomasz Wojdacz, Szczecin (PL); Jan Binkowski, Szczecin (PL); Olga Taryma-Lesniak, Szczecin (PL)

(21) Appl. No.: 19/163,034

(22) PCT Filed: Mar. 7, 2024
(86) PCT No.: PCT/IB2024/052219

§ 371 Date: Sep. 8, 2025

(30) Foreign Application Priority Data
Mar. 7, 2023 (PL) P.443998
Mar. 7, 2023 (PL) P.443999
Mar. 7, 2023 (PL) P.444000
Mar. 7, 2023 (PL) P.444001

(51) Int. Cl. G16B 40/00; G16B 50/10; G16B 50/50

(52) U.S. Cl. CPC G16B 40/00; G16B 50/10; G16B 50/50

(57) ABSTRACT

The subject of the invention is a computer implemented method for constructing profile of a tissue or a cell, the profile being based on pairs of adjacent epigenetically modified bases, based on epigenetic modification information derived from nucleic acid containing epigenetically modified bases-contained in a sample relating to said tissue or a cell, the method comprising the following steps: a) providing digital data on values of epigenetic modification level measurements for epigenetically modified bases contained in said nucleic acid in said sample relating to said tissue or said cell b) determining a set of pairs of adjacent epigenetically modified bases in said nucleic acid contained in said sample relating to said tissue or said cell, each pair of adjacent epigenetically modified bases consisting of two adjacent epigenetically modified bases localized within one nucleic acid molecule so as to generate a map of adjacent epigenetically modified bases containing genomic coordinates of each identified pair of adjacent epigenetically modified bases; c) compressing information on epigenetic modification level value, iteratively for each pair of adjacent epigenetically modified bases among the determined set of pairs of adjacent epigenetically modified bases, by converting two values of epigenetic modification level associated with two epigenetically modified bases in each pair, into one single numerical value, said single value representing compressed epigenetic modification information for a pair of adjacent epigenetically modified bases and being indicative of epigenetic modification status in each pair of adjacent epigenetically modified bases; d) saving genomic coordinates of adjacent epigenetically modified bases for each pair in said determined set of pairs and compressed epigenetic modification information in the form of one single numerical value for said each pair of adjacent epigenetically modified bases in said sample, so as to generate a profile based on pairs of adjacent epigenetically modified bases of said tissue or said cell.

### FIELD OF THE INVENTION

The invention relates to the field of epigenetics, especially to a method for analyzing specifically pre-processed epigenetic modification information contained in a raw epigenetic modification data. In particular, the methods for discriminating type of a biological cell based on analysis of methylation information pre-processed according to the invention have broad utility, for example, in identifying sample types, or for distinguishing between and among sample types, including different diseased cells or tissues. The invention also relates to methods for constructing tools for disease detection, i.e. a trained model for classification of cell or tissue type as well as a library of discriminative methylation profiles for a specific type of cell or tissue.

### BACKGROUND

Detection of abnormal cells is necessary in the process of the assessment of predisposition to the disease, disease diagnosis, treatment personalization and monitoring as well as post treatment surveillance. The detection of the abnormal cell can be based on any physical component of the cell, for example genetic material, epigenetic modifications of the genetic material or any of the biochemical substances that are produced by cells. In particular, because of relative specificity of epigenetic modifications for a type of cells, assessment of these modifications attracts more and more interest.

One type of epigenetic modification which has been explored intensively is DNA methylation. Covalent addition of methyl group to cytosine, referred to as DNA methylation, plays a key role in the regulation of gene expression (see Chan, M. F., Liang, G. and Jones, P. A. (2000) Relationship between transcription and DNA methylation. Curr Top Microbiol Immunol, 249, 75-86.).

Several studies have shown that the average methylation level varies within different genome regions and in particular can be different for a specific CpG site between different tissue types. Thus, further studies have been conducted to find practical methods to use the methylation information in diagnostics or clinical practice.

Methods for discriminating a type of a biological cell based on analysis of methylation data obtained from a wide variety of DNA samples taken from individuals are known in the art. In those methods, methylation status is measured for a single CpG site or a genomic region containing a number CpG sites and compared between different cells types, tissues or biological samples with the principles

of cases—control design. This allows to identify single CpG sites and/or regions with different methylation between compared cases and controls. The identified methylation differences allow to distinguish compared cases and controls as well as make a conclusion regarding the consequences of methylation differences by person skilled in the art (see Campagna M. P., et al. (2021) Epigenome-wide association studies: current knowledge, strategies and recommendations. Clin Epigenetics, 13(1):214.

US patent application US 2006/0183128 A1 discloses a method for generating a genome-wide epigenomic map, comprising a correlation between methylation variable CpG positions (MVP) and genomic DNA sample types. However, MVP are those CpG positions that show a variable quantitative level of methylation between sample types, and particularly, within the major histocompatibility complex (MHC). Particular genomic regions of interest (ROI) provide preferred marker sequences that comprise multiple, and preferably proximate MVP, and that have novel utility for distinguishing sample types. The epigenetic maps have broad utility, for example, in identifying sample types, or for distinguishing between and among sample types. In a preferred embodiment, the epigenomic map is based on methylation variable regions (MVP) within the major histocompatibility complex (MHC), and has utility, for example, in identifying the cell or tissue source of a genomic DNA sample, or for distinguishing one or more particular cell or tissue types among other cell or tissue types. Analysis of epigenetic characteristics of one, or of a set of nucleic acid sequences, in the context of an inventive epigenomic map, allows for the determination of an origin of the nucleic acids.

Moreover, US patent application US 2020/0407802 A1 relates to methods for measuring replication-associated genomic DNA methylation loss, using a Solo-WCGW DNA sequence motif (n(x)WCpGWn(x); wherein WIA or T, n=A or G or C or T and excludes any CG dinucleotides, and x>9) to filter the methylation data, more specifically for identification of common PMDs, partially methylated domains, shared between normal tissue types, or specific to individual normal or diseased tissue types.

Furthermore, a deconvolution models trained to generate source of origin predictions for early detection of cancer in subjects are also known in the art. For example, US patent application US 2020/0239965 A1 discloses a method and system for determining one or more sources of a cell free deoxyribonucleic acid (cfDNA) test sample from a test subject. The cfDNA test sample contains a plurality of deoxyribonucleic acid (DNA) molecules with numerous CpG sites that may be methylated or unmethylated. A trained deconvolution model comprises a plurality of methylation parameters, including a methylation level at each CpG site for each source, and a function relating a sample vector as input and a source of origin prediction as output. The method generates a test sample vector comprising a site methylation metric relating to DNA molecules from the test sample that are methylated at that CpG site. The method inputs the test sample vector into the trained deconvolution model to generate a source of origin prediction indicating a predicted DNA molecule contribution of each source.

Also, the publication of international application WO 2017/106481 provides a method for distinguishing an aberrant methylation level for DNA from a first cell type, including steps of (a) providing a test data set that includes (i) methylation states for a plurality of sites from test genomic DNA from at least one test organism, and (ii) coverage at each of the sites for detection of the methylation states; (b) providing methylation states for the plurality of sites in reference genomic DNA from one or more reference individual organisms, (c) determining, for each of the sites, the methylation difference between the test genomic DNA and the reference genomic DNA, thereby providing a normalized methylation difference for each site; and (d) weighting the normalized methylation difference for each site by the coverage at each of the sites, thereby determining an aggregate coverage-weighted normalized methylation difference score. Also provided herein are sensitive methods for using genomic DNA methylation levels to distinguish cancer cells from normal cells and to classify different cancer types according to their tissues of origin.

In the US patent application US 2020/0131582 A1 there are disclosed methods and systems which use sequencing reads for detecting and quantifying the presence of a tissue type or a disease type in cell-free DNA prepared from blood samples. A new way to differentiate disease-specific cfDNA reads from normal cfDNA reads was proposed. When the methylation levels of all CpG sites in a given

read (denoted a-value) are averaged, there is a striking difference (0 and 1) between the abnormally methylated cfDNAs and the normal cfDNAs (atumor=0% and anormal=100%). In other words, given the pervasive nature of DNA methylation, the joint methylation patterns of multiple adjacent CpG sites can easily distinguish cancer-specific cfDNA reads from normal cfDNA reads. Inspired by the a-value, it was realized that the key to exploiting pervasive methylation is to estimate whether the joint probability of all CpG sites in a read follows the DNA methylation signature of a disease.

However, still said known methods show drawbacks, e.g. they provide complex methylation signatures what leads to models over-fitting and due to amount of required data reduce potential utility in clinical application. Moreover, these methods are prone to be affected by unwanted technical variance for example—batch-effect.

### OBJECT OF THE INVENTION

The object of the invention is to propose an alternative method to a known problem which is discrimination of type of a biological cell or tissue using the epigenetic modification information contained in its DNA material in a credible and efficient manner. Moreover, the object of the invention is to propose a method for discrimination of type of a biological cell or tissue which would be less time consuming and more credible. Finally, the object of the invention is to propose a method of discriminating type of a biological cell or tissue that could be easily used in practice for detection and classification of different healthy and pathological cells or tissues.

This disclosure discloses different embodiments of methods, apparatuses, computer products for screening and identifying the tissue-of-origin of cells using any type of samples drawn from patients.

### SUMMARY OF THE INVENTION

According to a first aspect, the invention provides a computer implemented method for constructing profile of a tissue or a cell, the profile being based on pairs of adjacent epigenetically modified bases, based on epigenetic modification information derived from nucleic acid containing epigenetically modified bases-contained in a sample relating to said tissue or a cell, the method comprising the following steps:
a) providing digital data on values of epigenetic modification level measurements for epigenetically modified bases contained in said nucleic acid in said sample relating to said tissue or said cell
b) determining a set of pairs of adjacent epigenetically modified bases in said nucleic acid contained in said sample relating to said tissue or said cell, each pair of adjacent epigenetically modified bases consisting of two adjacent epigenetically modified bases localized within one nucleic acid molecule so as to generate a map of adjacent epigenetically modified bases containing genomic coordinates of each identified pair of adjacent epigenetically modified bases;
c) compressing information on epigenetic modification level value, iteratively for each pair of adjacent epigenetically modified bases among the determined set of pairs of adjacent epigenetically modified bases, by converting two values of epigenetic modification level associated with two epigenetically modified bases in each pair, into one single numerical value, said single value representing compressed epigenetic modification information for a pair of adjacent epigenetically modified bases and being indicative of epigenetic modification status in each pair of adjacent epigenetically modified bases;
d) saving genomic coordinates of adjacent epigenetically modified bases for each pair in said determined set of pairs and compressed epigenetic modification information in the form of one single numerical value for said each pair of adjacent epigenetically modified bases in said sample so as to generate as a profile based on pairs of adjacent epigenetically modified bases of said tissue or said cell.

Advantageously, the epigenetic modification status is one among at least co-epigenetic modification status and non co-epigenetic modification status in a pair of adjacent epigenetically modified bases.

Advantageously, epigenetic modification comprises one among methylation, hydroxymethylation, formylation or carboxylation, and the epigenetically modified base of the nucleic acid sequence is respectively a methylated base, a hydroxymethylated base, a formylated base, or a carboxylic acid containing base or a derivative thereof.

Advantageously, the step c) involves calculating epigenetic modification value difference between two epigenetically modified bases in each pair of said adjacent epigenetically modified bases.

Advantageously, the step b) comprises first a step of annotating said nucleic acid containing epigenetically modified base to a reference genome thereby determining genomic coordinates of each epigenetically modified base so as to generate a map of all epigenetically modified bases.

Advantageously, each said pair of adjacent epigenetically modified bases is further localized at a distance less than a pre-defined threshold distance.

Advantageously, the pre-defined threshold distance is less than 50 bp.

Advantageously, the step a) comprises providing said sample, extracting nucleic acid fragments from said sample, converting said extracted nucleic acid fragments, assessing epigenetic modification levels in said converted isolated nucleic acid so as to receive a plurality of values of epigenetic modification level measurements for said sample thereby providing digital data on values of epigenetic modification level measurements for epigenetically modified bases.

Advantageously, the step a) comprises downloading data from publicly available databases.

According to a second aspect, the invention provides a computer implemented method for constructing parametrized profile based on pairs of adjacent epigenetically modified bases of a known type of tissue or cell based on epigenetic modification information derived from epigenetically modified base-containing nucleic acid contained in a group of at least one sample, each sample relating to the same known type of said tissue or said cell, the method comprising:
a) for each sample in said group of at least one sample providing digital data on epigenetic modification level measurements for epigenetically modified bases contained in said nucleic acid in said sample relating to said tissue or said cell of a known type
b) determining a set of pairs of adjacent epigenetically modified bases in said nucleic acid contained in each sample in said group of at least one sample relating to said tissue or said cell of a known type, each pair of adjacent epigenetically modified bases consisting of two adjacent epigenetically modified bases localized within one nucleic acid molecule so as to generate a map of adjacent epigenetically modified bases containing genomic coordinates;
then c) for said group of at least one sample iteratively for each pair of adjacent epigenetically modified bases among the determined set of pairs of adjacent epigenetically modified bases, compressing information on epigenetic modification level value, by fitting a parametrized mathematical model to said digital data on values of epigenetic modification level measurements associated with each pair of adjacent epigenetically modified bases so as to determine one single model parameter, said single model parameter representing compressed epigenetic modification information for a pair of adjacent epigenetically modified bases and being indicative of epigenetic modification status in each pair of adjacent epigenetically modified bases for said group of at least one sample;
e) for said group of at least one sample saving genomic coordinates of adjacent epigenetically modified bases for each pair in said determined set of pairs and compressed epigenetic modification information in the form of one model parameter for said each pair of adjacent epigenetically modified bases in said group of at least one sample
   so as to generate said parametrized profile based on pairs of adjacent epigenetically modified bases of a known type of tissue or cell.

Advantageously, the epigenetic modification status is one among at least co-epigenetic modification status and non co-epigenetic modification status in a pair of adjacent epigenetically modified bases.

Advantageously, the step c) comprises:
for each pair of adjacent epigenetically modified bases separately, estimating a regression model such that:

$$MetLevel ∼ intercept + {β}_{i} * POS$$

$$POS = \{ \begin{matrix}0 & if CpG = first \\ 1 & if CpG = successive\end{matrix}$$

wherein the intersection point (intercept) and βi are model parameters, the parameter POS being an exogenous variable taking values of 0 if the epigenetically modified base in a pair is a first one, or 1 if the epigenetically modified base in a pair is a successive one.

Advantageously, if βi≠0 the non co-epigenetic modification status is further one among negative sign non co-epigenetic modification status for βi<0 and positive sign non co-epigenetic modification status for βi>0.

Advantageously, the statical significance assessment of the value of the βi parameter is performed using Student's test to calculate and save the value of empirical probability (p-value).

Advantageously, the step c) comprises
a) determining, separately for each first and each successive epigenetically modified base in each same pair of adjacent epigenetically modified bases across said group of at least one sample, a confidence interval for the mean epigenetic modification level such that:

$$P ( \overline{X} - {t}_{\text{?}} \frac{S}{\sqrt{n}} < μ < \overline{X} + {t}_{\text{?}} \frac{S}{\sqrt{n}} ) = 1 - \text{?} ,$$

$$\text{?} \text{indicates text missing or illegible when filed}$$

wherein alfa is a predefined parameter, advantageously equal to 0.05, n—the number of samples in the set of samples, S—standard deviation from the epigenetic modification level in said group of samples for each epigenetically modified base in the pair, t 1-alfa/2 the quantile of order 1-alfa/2 of student's t-distribution with n−1 degrees of freedom.
b) determining a model parameter being the distance βi between the confidence interval calculated for the first epigenetically modified base and the confidence interval calculated for the successive epigenetically modified base in the pair using a selected measure of distance.

Advantageously, wherein the selected measure of distance βi is:
the Chebyshev measure:

$${D}_{Chebyshev} ( x , y ) := {max}_{i} ( \vert {x}_{i} - {y}_{i} \vert )$$

Advantageously, the step c) comprises
a) determining, separately for each first and each successive epigenetically modified base in each same pair of adjacent epigenetically modified bases across said group of samples, mean or median level of epigenetic modification
b) determining a model parameter βi being the absolute value of difference between mean levels of epigenetic modification of the epigenetically modified bases for the first epigenetically modified base and the successive epigenetically modified base.

Advantageously, it further comprises testing significance of said calculated difference between mean levels of epigenetic modification of the epigenetically modified bases using statistical tests such as t-test, ANOVA, Mann-Whitney U or Kruskal-Wallis to calculate and save the value of empirical probability (p-value) for each pair of adjacent epigenetically modified bases.

According to another aspect, the invention provides a computer implemented method for constructing a discriminative map of cells or tissues types based on pairs of adjacent epigenetically modified bases of a known cell type or tissue type within a specific application context, the method comprising:
a) providing at least two parametrized profiles based on pairs of adjacent epigenetically modified bases for at least two different known types of cells or tissues generated with the method according to any of claim 10 to 18, wherein the number and the known types of cells or tissues for which the parametrized profiles are provided giving a specific application context.
b) determining and comparing the epigenetic modification status contained in the model parameters for each pair of adjacent epigenetically modified bases present in all of said at least two parametrized profiles
c) extracting each pair to be a part of said discriminative map of cells or tissues types under construction if the epigenetic modification status in at least one parametrized profile for said pair is different from the epigenetic modification information status in at least one among all other parametrized profiles.
d) saving genomic coordinates for extracted pairs of adjacent epigenetically modified bases so as to generate said discriminative map of cells or tissues types for a specific application context.

Advantageously, in the step b) it is determined that
if βi is approximately equal to 0, then the epigenetic modification level of both epigenetically modified bases in the pair is the same and the epigenetic modification status for said pair is co-epigenetic modification status or that
if the absolute value of the parameter βi>0, then the epigenetic modification level of both epigenetically modified bases in the pair is different and the epigenetic modification status for said pair is non co-epigenetic modification status.

Advantageously, in the step b) if the parameter p-value is available and the p-value is higher than a statistical significance threshold then it is determined that βi is equal to 0 then the epigenetic modification level of both epigenetically modified bases in the pair is the same and the epigenetic modification status for said pair is co-epigenetic modification status.

Advantageously, in the step b) the non co-epigenetic modification status is determined for |βi|>p threshold value, wherein the p threshold value being a value in the range from 0 to 1 excluding 0 and 1.

Advantageously, in the step c) a pair of adjacent epigenetically modified bases is extracted to be a part of said discriminative map of cells or tissues types under construction if the epigenetic modification status in one parametrized profile for said pair is a non co-methylation status and the epigenetic modification status in all other parametrized profiles is a co-methylation status.

Yet, according to another aspect, the invention provides a computer implemented method for constructing cell or tissue type discriminative profile based on pairs of adjacent epigenetically modified bases of nucleic acid contained in a sample relating to a certain type of tissue or cell within a specific application context, the method comprising:
a) for said sample relating to a certain type of tissue or cell providing its profile based on pairs of adjacent epigenetically modified bases, said profile being generated with the use of the method according to any of claim 1 to 9,
b) providing a discriminative map of cells or tissues types based on pairs of adjacent epigenetically modified bases, said map being generated for said specific application context with the use of the method according to claim 19,
c) saving, only for pairs of adjacent epigenetically modified bases contained in said discriminative map of cell or tissues types,
   genomic coordinates of adjacent epigenetically modified bases and—compressed epigenetic modification information in the form of one single numerical value relating to said adjacent epigenetically modified bases, so as to generate said cell or tissue type discriminative profile based on pairs of adjacent epigenetically modified bases for said sample relating to a certain type of tissue or cell within said specific application context.

Yet, according to another aspect, the invention provides a computer implemented method for providing a model for cell or tissue type classifying, the method comprising:
a) providing a training data set by
   providing a first set of cell or tissue type discriminative profiles based on pairs of adjacent epigenetically modified bases within a specific application context for the first known type of the tissue or cell along with associated labels indicating the first known type of tissue or cell
   providing at least a second set of cell or tissue type discriminative profile based on pairs of adjacent epigenetically modified bases within a specific application context for at least the second known type of the tissue or cell along with associated labels indicating the at least second known type of tissue or cell, the first and at least the second set of cell or tissue type discriminative profiles being obtained by the method according to claim 24;
b) training the classification model with said training data set so as it is configured to classify the type of a cell or tissue relating to an input cell or tissue type discriminative profile.

Yet, according to another aspect, the invention provides a computer implemented method of classifying a tissue or cell of origin contained in a test sample, the method comprising:
a) for said test sample providing a discriminative profile based on pairs of adjacent epigenetically modified bases within a specific application context, said discriminative profile for said test sample being received by the method according to claim 24;
b) inputting said discriminative profile based on pairs of adjacent epigenetically modified bases for said test sample into a trained classification model;
c) receiving a result of the classification that indicates the tissue or cell of origin contained in said test sample;

Yet, according to another aspect, the invention provides a computer implemented method of constructing reference discriminative profile of a known cell or tissue type based on pairs of adjacent epigenetically modified bases within a specific application context, the method comprises:
a) providing a set of discriminative profiles based on pairs of adjacent epigenetically modified bases within said specific application context obtained by the method according to claim 24 for a group of at least one sample of the same known type of the tissue or cell;
b) for each pair of adjacent epigenetically modified bases in said discriminative profiles, for all samples in said group of at least one sample of the same cell or tissue type, calculating one among a mean value or median resulting from a set of compressed epigenetic modification information in the form of one single numerical value relating to said each pair for said all samples in said group of at least one samples;
c) saving genomic coordinates of each pair of adjacent epigenetically modified bases and said calculated mean value or median of the compressed epigenetic modification information in the form of one single numerical value for said each pair so as to generate a reference discriminative profile of a known cell or tissue type based on pairs of adjacent epigenetically modified bases within a specific application context.

Yet, according to another aspect, the invention provides a computer implemented method for generating a library of reference discriminative profiles of cell or tissue types based on pairs of adjacent epigenetically modified bases of known types of tissue or cell for specific application, the method comprising:
a) providing a first reference discriminative profile of cell or tissue types based on pairs of adjacent epigenetically modified bases within a specific application context for a first known type of cell or tissue; b) providing at least a second reference discriminative profile of cell or tissue types based on pairs of adjacent epigenetically modified bases within a specific application context for at least a second known type of cell or tissue, said first and at least second reference discriminative profiles of cell or tissue types being generated with the method according to claim 27;
c) storing said at least two reference discriminative profiles of cell or tissue types based on pairs of adjacent epigenetically modified bases so as to generate said library of reference discriminative profiles.

Yet, according to another aspect, the invention provides a computer implemented method for determining a tissue or cell of origin contained in a test sample, the method comprising:
a) providing a library of reference discriminative profiles of cell or tissue types based on pairs of adjacent epigenetically modified bases, the library being obtained by the method according to claim 28; b) providing a querying tool;
c) providing for said test sample a discriminative profile based on pairs of adjacent epigenetically modified bases, said discriminative profile being received by the method according to claim 24
d) querying said library of reference discriminative profiles with the use of the querying tool and said discriminative profile of said test sample; and
e) receiving a result of the query that indicates the tissue or cell of origin contained in said test sample.

Yet, according to another aspect, the invention provides a computer implemented method for constructing reduced cell or tissue type discriminative profile based on pairs of adjacent epigenetically modified bases of known types of tissue or cell within a specific application context:
a) providing the classification model received by the method according to claim 25;
b) providing a training data set being a first and at least a second set of cell or tissue type discriminative profiles based on pairs of adjacent epigenetically modified bases for at least two different types of cell or tissue obtained by the method according to claim 24 along with associated labels indicating the known type of tissue or cell;
c) eliminating unnecessary pairs of adjacent epigenetically modified bases in the training data set by further training said classification model with the use of a feature selection method;
d) saving reduced discriminative map;
e) saving said further reduced trained model.

Yet, according to another aspect, the invention provides a computer implemented method for predicting a sample composition of a test sample, the method comprising:
a) providing a library of reference discriminative profiles of cell or tissue types based on pairs of adjacent epigenetically modified bases, the library being obtained by the method according to claim 28;
b) providing a deconvolution tool;
c) providing for said test sample a discriminative profile based on pairs of adjacent epigenetically modified bases, said discriminative profile being received by the method according to claim 24
d) performing deconvolution with the use of the deconvolution tool based on data contained in the library of reference discriminative profiles and contained in said discriminative profile of the test sample; and
e) receiving a result of the deconvolution that indicates the composition of said test sample.

Yet, according to another aspect, the invention provides a computer-readable storage medium having instructions encoded thereon which when executed by a processor, cause the processor to perform the method according to claim 1 to 9 or the method according to claim 10 to 18 or the method according to any of claim from 19 to 31.

Yet, according to another aspect, the invention provides a computer program product comprising a computer-readable medium having computer program logic recorded thereon arranged to put into effect any method according to claim 1 to 9 or the method according to claim 10 to 18 or the method according to any of claim from 19 to 31.

### Advantages of the Invention

The present invention provides high-precision approach that facilitates accurate prediction and assessment of the risk of multiple cancers, and is free from the drawbacks that are known from the state of the art. Some of the advantages of the invention will be further characterized.

The invention provides an easier, more credible and less time-consuming method for discriminating a type of biological cell thanks to an alternative processing and analysis of epigenetic modification information, namely thanks to analysis of relative epigenetic modification level differences contained in two adjacent CpG sites. The authors of the invention proposed an unexpected way of encoding the status of mutual relation of the methylation level for two CpG sites in a pair in a compressed and interpretable form, namely as one single numerical value, although the status in a pair can be easily interpreted also with the use of a typical graph presentation with the use of the raw data. Said one single numerical value can be a result of any mathematical conversion of two methylation level values that is interpretable for that purpose. Advantageously, calculation of the difference between said two methylation level in a pair is an example of a mathematical conversion useful for said interpretation purpose.

Analysis of epigenetic modification value difference within specific CpGs pairs, which are the smallest possible regions of CpG sites, allows to catch simple and compressed methylation pattern encoded in CpG pair-based methylation statuses of co-methylation and non co-methylation, specific for cell- or tissue-type, which is very distinctive and discriminative. The invention, allows to reduce number of predictors necessary to perform many types of cancer tissue classification to less than few hundreds.

It also allows to compare DNA epigenetic modification data corresponding to a diversity of genomic DNA sources and conditions (e.g., corresponding to different isolation methods, different efficiencies of bisulfite pretreatment of the DNA, different amplification/PCR conditions). In particular, by processing and analyzing methylation data for consecutive pairs of adjacent CpG sites the methods according to the invention allow reliably detect and classify different healthy and pathological cells or tissues based on analysis of DNAs, in particular cfDNAs.

The conversion of epigenetic modification data into the form of a set of relative values of the epigenetic modification levels between CpG sites in each pair of adjacent CpGs allows to obtain clear and easily interpretable epigenetic modification signatures including potential methylation based markers, suitable for easy and efficient comparison with other such epigenetic modification signatures. The conversion of epigenetic modification data into the form of a set of relative values of the epigenetic modification level between CpG sites in each pair of adjacent CpGs results in a change of scale. In particular, delta methylation level (difference of epigenetic modification levels in a pair of adjacent CpG sites) is a single value between −1 and 1 which additionally allows to interpret a direction of changes in methylation levels within a pair of CpG sites.

Additionally, by preforming an automated selection of methylations markers, the present invention makes it possible to further reduce the number of methylation markers for the purpose of laboratory application.

Moreover, thanks to the simplicity of methylation signatures, a more precise estimation of cell types proportion (deconvolution) in a biological sample is obtained. When comparing efficiency of deconvolution algorithms with respect to different known reference atlases, the CpG pairs-based reference discriminative profiles according to present invention allow more precise cell proportion estimation (deconvolution).

The methylation signatures according to the invention consist of a set of two types of statuses, namely co-epigenetic or non co-epigenetic modification in a CpG pair. The received signatures are suitable for interpretation even directly from raw data. As a consequence, it is possible to generate discriminative CpG pair-based profiles for different tissues and cells which do not require complex processing of data relating to test samples in order to asses them. Moreover, as mentioned earlier, said discriminative CpG pair-based profiles for different tissues and cells are favorable for training automatic classifiers.

It should be emphasized that assaying for the co-methylation or non co-methylation may help to overcome two major technological limitations that methylation screening technologies suffer from.

Firstly, information about non co-methylation or co-methylation status of CpG pair is derived from comparison of methylation levels within one biological replicate. This overcomes the challenges of batch effects. The batch effects confound methylation levels at the consecutive CpG sites equally, thus can be to large extent neglected in the analysis of the methylation of non co-methylated and co-methylated CpG sites.

Secondly, targeting non co-methylation or co-methylation may significantly reduce the lengths of the genomic region for which information about methylation of CpG sites is required to identify the origin of the DNA template, thus problems with measurements quality are irrelevant to the methods according to the invention. This is especially important when identifying clinically or diagnostically relevant epigenetic modification changes, in particular methylation changes, in highly degraded material such as FFPE (Formalin Fixed Paraffin Embedded) tissues or templates extracted from liquid biopsies.

A better understanding of the nature and advantages of embodiments of the present invention may be gained with reference to the following detailed description and the accompanying drawings.

### BRIEF DESCRIPTION OF THE DRAWING

The enclosed drawings should illustrate embodiments of the present invention and convey a further understanding thereof. In connection with the description, they serve as explanation of concepts and principles of the invention. Other embodiments and many of the stated advantages can be derived in relation to the drawings. The elements of the drawings are not necessarily to scale towards each other. Identical, functionally equivalent and acting equal features and components are denoted in the figures of the drawings with the same reference numbers, unless noted otherwise.

FIGS. 1a-1c show known co-methylation and non co-methylation phenomena across certain regions in human cells;

FIGS. 2a-2b show two types of co-methylation status that can be observed with new resolution in CpG pairs;

FIGS. 3a-3c present three types of non co-methylation status that can be observed with new resolution in CpG pairs;

FIG. 4a shows schematic illustration of the identification procedure of all pairs of CpG spaced less than a certain arbitrary threshold value, for example, 50 bp in the genome;

FIG. 4b presents schematic illustration of the difference and compatibility in methylation status in pairs between different types of samples as well as the concept of choosing pairs for new methylation signatures;

FIG. 5 shows a heat map of correlation between the methylation levels of CpGs placed in specific distance, estimated based on 10 000 randomly selected CpG sites;

FIGS. 6a-6b show clustered heat maps of methylation status of adjacent CpG sites in 112 healthy blood samples;

FIGS. 7a-7d present the Sanger sequencing chromatograms for selected 4 CpG pairs in exemplary sample of DNA extracted from whole blood cell.

FIGS. 8a-8c present raw data from quantitative measurements for the same selected 4 CpG pairs in the same exemplary sample of DNA extracted from whole blood;

FIG. 9 illustrates a diagram of absolute methylation level difference change per year in pairs of CpGs in the set of CpG pairs measured for 112 healthy blood samples;

FIG. 10 shows a heat map of difference value of methylation levels in identified pairs of CpG sites in different types of blood cells;

FIGS. 11a-11f are plots of methylation levels for each CpG in selected CpG pairs for said different types of blood cells for several samples; said plots corresponding to chosen boxes of the heat map of methylation status shown in FIG. 10;

FIG. 12 shows a Jaccard similarity index matrix for blood cell fractions representing an overlap of the CpG pairs with non co-methylation status between each two fraction types;

FIG. 13 illustrates a heat map of difference value of methylation levels in identified CpG pairs in healthy blood, AML and CLL;

FIGS. 14a-14f are plots of methylation levels for each CpG in selected CpG pairs for said different types of samples, said plots corresponding to chosen boxes of the heat map of methylation status shown in FIG. 13;

FIG. 15 presents comparison of standard deviation (STD) of delta beta-values for selected CpG pairs between healthy blood, AML, and CLL shown in FIG. 13;

FIG. 16 are plots of methylation levels for each CpG in two exemplary CpG pairs among 64 pairs identified not to change non co-methylation status in two cancers (CLL, AML) and five healthy tissues (whole blood, bone marrow, colon, breast and skeleton muscle tissues);

FIG. 17 illustrates a heat map of difference value of methylation levels in identified CpG pairs in naive and mature subtypes of B cell, as well as in CLL samples with mutated (CLL IGHV 1) and unmutated IGHV (CLL IGHV 0) status;

FIG. 18 presents a heat map of difference value of methylation levels in identified pairs of CpG sites in whole blood, bone marrow, skeletal muscle, colon, and breast tissue;

FIG. 19 shows an overlap matrix of identified CpG pairs having non co-methylation status between different types of healthy tissues. Each Box represents the Jaccard similarity index representing fraction of CpG pairs having non co-methylation status shared between each of the two tissues;

FIG. 20 presents an exemplary overview of processing and analysis of methylation data with a novel “CpG pair-based resolution” based on three components of the present invention;

FIG. 21 illustrates the computer implemented method for constructing CpG pair-based profile of a tissue or a cell according to the invention;

FIG. 22 shows an exemplary known step of providing data on methylation level at specific CpG sites for at least a part of a genome performed on at least one biological sample;

FIG. 23a-23b present, in the form of box and scatter plots, difference values of methylation levels in an exemplary CpG pair vs values of methylation levels for a single CpG site constituting said exemplary CpG pair for pathological and healthy types of tissues to be differentiated;

FIG. 24a-24b present, in the form of box and scatter plots, difference values of methylation levels in an exemplary CpG pair vs value of methylation level for a single CpG site constituting said exemplary CpG pair for two different pathological types of tissues to be differentiated;

FIG. 25 presents an exemplary overview of the method for constructing parametrized profile 30 based on pairs of adjacent epigenetically modified bases of a known type of tissue or cell according to the invention;

FIG. 26 illustrates an exemplary overview of the method for providing a discriminative map of cells or tissues 40 based on pairs of adjacent epigenetically modified bases of a known cell type or tissue type within a specific application context;

FIG. 27a-27b shows a visualization of discriminative “non co-metylated” status and discriminative “co-methylated” status (plots of raw data on methylation levels) for chosen CpG pairs across two different types of cancer of white blood cells;

FIG. 28 presents an exemplary overview of the computer implemented method for providing cell or tissue type discriminative profile 50 based on pairs of adjacent epigenetically modified bases of nucleic acid contained in a sample within a specific application context hidden in the discriminative map 40;

FIG. 29 presents an exemplary overview of the method for providing cell or tissue type reference profile 60 based on pairs of adjacent epigenetically modified bases of nucleic acid according to the invention;

FIG. 30 illustrates an exemplary overview of the method for providing a trained classification model 70 according to the invention;

FIG. 31 illustrates an exemplary overview of method constructing reduced classification model 70′ within a specific context application;

FIG. 32 presents graphically as heatmaps a full set of identified CpG pairs in the set of samples of 3 different cell types and appropriate discriminative subset of CpG pairs within said specific context application;

FIG. 33 shows an exemplary overview of the method for providing a library 80 of reference discriminative profiles 60 for given cell or tissue types as well as an automated tool for cell or tissue typing;

FIG. 34 illustrates an exemplary overview of the method for providing an automated tool for sample composition typing;

FIG. 35 shows an exemplary overview of the computer implemented method of classifying a tissue or cell of origin contained in a test sample with the use of trained classification model according to the invention;

FIG. 36 illustrates an exemplary overview of the computer implemented method for determining a tissue or cell of unknown origin contained in a test sample according to the invention;

FIG. 37 shows an exemplary overview of the computer implemented method for predicting a sample composition of an unknown test sample according to the invention;

FIG. 38 shows a schematic diagram of the computer system configured to perform all methods according to the invention

FIG. 39a illustrates a heat map of methylation status in 58 identified CpG pairs constituting reference profiles 60 for healthy lung, lung adenomas and adenocarcinomas tumours and lung squamous cell neoplasms tumours;

FIG. 39b shows the performance metrics of exemplary searchable library 80 for which reference discriminative profiles 60 containing said 58 CpG pairs were used;

FIG. 40 shows a graph illustrating ratio of false positive cases received with Kruskal-Walli's test for measurements at single CpG sites and for the difference value of methylation levels in CpG pairs calculated according to the invention;

FIG. 41 illustrates by way of comparison of number of clusters the lower influence of the batch effect on samples for which the difference value of methylation levels in CpG pairs-based profiles was calculated according to the invention;

FIG. 42 shows a workflow for training an efficient reduced classification model 70′ based on data containing less than 200 biomarkers according to the invention;

FIG. 43 shows the performance metrics of the received reduced classification model 70′;

FIG. 44 illustrates the difference value of methylation levels in exemplary CpG pairs of 5 reference profiles 60 contained in an exemplary reference library 80 built according to the invention;

FIG. 45 shows a heat map visualization of delta methylation levels used to create 5 reference profiles 60 contained in said reference library 80 built according to the invention;

FIG. 46 presents rescaling operation performed so that the actual proportions of cells in the sample estimated using the FACS method were comparable to those proportions estimated using deconvolution algorithms.

FIG. 47 illustrates calculation of median of absolute residuals per each sample

FIG. 48 shows a plot of estimation residuals for the results of the automated composition typing tool 90 in which 3 different deconvolution algorithms were used as well as two different reference libraries, namely said exemplary reference library 80 according to the invention and a known default reference library;

FIG. 49 presents in numbers a median of residuals presented on FIG. 45;

FIG. 50 illustrates an exemplary reference atlas 80 for deconvolution containing reference profiles 60 for three different tumour origin tissue types that can be used for cf-DNA typing;

FIGS. 51A-D present exemplary CpG pairs for which the methylation level difference is correlated with the biological age;

FIG. 52 shows statistics (Pearson correlation coefficient, p-value, regression line slope coefficient, regression line intercept coefficient) describing the association between age and delta methylation values for 5 selected CpG pairs not stable in lifespan;

FIG. 53 is a table showing mean absolute error between predicted age and chronological age in function of CpG pairs number used for prediction;

### DETAILED DESCRIPTION OF THE INVENTION

The present invention will now be described in detail. First, appropriate definitions are provided. Next, background studies are presented. Finally, a detailed description of all components of the way of industrial application of the present invention is provided. Only exemplary implementations of the present application are shown and described in the detailed description below. As will be appreciated by those skilled in the art, the contents of this disclosure enable those skilled in the art to make changes to the disclosed detailed embodiments without departing from the spirit and scope of the inventions to which this application relates. Accordingly, the description in the drawings and detailed description is merely exemplary and not limiting.

### Definitions

The present technology is described herein using several definitions, as set forth throughout the specification. Unless defined otherwise, all technical and scientific terms used herein generally have the same meaning as commonly understood by one of ordinary skill in the art to which this invention belongs. As used herein, unless otherwise stated, the singular forms “a,” “an,” and “the” include plural reference. Thus, for example, a reference to “a nucleic acid” is a reference to one or more nucleic acids.

As used herein, the term “allele” is intended to be a genetic variation associated with a segment of DNA, i.e., one of two or more alternate forms of a DNA sequence occupying the same locus.

In the present application, the term “human reference genome” generally refers to a human genome that can perform a reference function in gene sequencing. Information about the human reference genome may be for example accessed from the Ensembl database. Due to ongoing process of sequencing of human genome, the human reference genome is periodically updated and thus the reference genome may have different versions, e.g., may be hg 19, GRCH38 or T2T.

The term “biological sample” or “test sample” as used herein, refers to, but is not limited to, any biological sample derived from, or obtained from, a subject. The sample may comprise nucleic acids, such as DNAs or RNAs. In some embodiments, samples are not directly retrieved from the subject, but are collected from the environment, e. g. a crime scene or a rape victim. Examples of such samples include but are not limited to fluids, tissues, cell samples, organs, biopsies, etc. Suitable samples include but are not limited to blood, plasma, saliva, urine, sperm, hair, etc. The biological sample can also be blood drops, dried blood stains, dried saliva stains, dried underwear stains (e.g. stains on underwear, pads, tampons, diapers), clothing, dental floss, ear wax, electric razor clippings, gum, hair, licked envelope, nails, paraffin embedded tissue, post mortem tissue, razors, teeth, toothbrush, toothpick, dried umbilical cord. Genomic DNA can be extracted from such samples according to methods known in the art. (for example using a protocol from Sambrook et al., Molecular Cloning: A Laboratory Manual, Second Ed., Cold Spring Harbor Laboratory Press, Cold Spring Harbor, NY, 1989).

The term “epigenetic modification” as used herein refers to the heritable phenotype changes that are independent of nucleic acid sequence changes and affect the function of a locus or chromosome without altering the underlying nucleic acid sequence. In particular, epigenetic modifications include a variety of molecular mechanisms such as DNA methylation and histone modification that change the expression of a gene.

The term “epigenetically modified base” refers to a base such as nucleobase which has undergone “epigenetic modification”. The term “epigenetically modified base” comprises the term “methylated CpG” and herein means a CpG with attached methyl group.

The term “non-epigenetically modified base” refers to a base such as nucleobase which has not undergone “epigenetic modification”. The term “non epigenetically modified base” comprises the term “non-methylated CpG” and means CpG without attached methyl group.

The term “CpG” or “CpG site” as used herein means a CG dinucleotide in which cytosine can undergo a methyl group attachment reaction. There are approximately 2.8*10⁷ CG dinucleotides in the human genome, but for the purposes of simplifying, the term CpG is used in the context of a specific CpG in a specific region of the genome.

The term “co-epigenetic modification” is defined as a state/status in a pair of epigenetically modified bases for which epigenetic modification level at both base sites, namely first epigenetically modified base and successive epigenetically modified base, is equal.

The term “non co-epigenetic modification” is defined a state/status in a pair of epigenetically modified bases for which epigenetic modification level at both base sites, namely first epigenetically modified base and successive epigenetically modified base, is not equal.

The term “co-epigenetically modified bases' pair” comprises the term “co-methylated CpGs' pair” which is defined as CpGs' pair for which methylation level at both CpG sites in CpGs' pair is equal.

The term “non co-epigenetically modified bases' pair” comprises the term “non co-methylated CpGs' pair” which is defined as CpG pair for which methylation level at both CpG sites in CpG pair is not equal. It also comprises the term “discordantly methylated CpG pair” which is an alternative term for indicating CpG pair for which methylation level at both CpG sites in CpG pair is not equal.

The term “epigenetic modification level” comprises the term “methylation level” and is the most general term for the result of the methylation measurement for one CpG site. It relates to the proportion of nucleic acid molecules containing methylation in particular genomic coordinates. It relates to both types of measurements, namely quantitative as well as qualitative. The term “methylation level” encompasses the term “methylation rate”/“methylation ratio/methylation density/methylation signal intensity” which are used for designating results of quantitative measurements as well as “methylation status/methylation state” which are used for qualitative measurements.

The term “methylation state” or “methylation status”, as used herein, generally refers to the presence or absence of 5-methylcytosine (“5-mC”) at one or a plurality of CpG dinucleotides within a DNA sequence. Methylation states at one or more particular palindromic CpG methylation sites (each having two CpG dinucleotide sequences) within a DNA sequence include “unmethylated,” “fully-methylated”, and “hemi-methylated.”

For example, in case of NGS-based technology methylation level is calculated using methylation rate/ratio and it is a ratio of methylated cytosine(s) and the total number of cytosine(s) found in the sequencing reads obtained for the specific genomic region.

In case of hybridization-based methods, such as single base extension reaction in e.g. microarray-based technology, methylation level is calculated using a ratio between signal intensity from probe assaying the methylated nucleobase and from probe assaying the un-methylated nucleobase and methylated nucleobase in specific genomic position. A suitable method is, for example, the one described by Illumina, Inc in Infinium MethylationEPIC kit.

The term “adjacent epigenetically modified bases within a pair” comprises the term “adjacent CpGs within the pair” or “a pair of adjacent CpGs” or “adjacent CpGs' pair” as used herein is a pair of consecutive and closest measured CpGs that are separated by a certain distance expressed in base pairs [bp].

The term “epigenetically modified bases pairs-based map” comprises the term “CpGs' pairs-based methylation map” as used herein and means a set of localization information of all pairs of adjacent CpG found within at least a part of a genome derived from nucleic acid.

The term ‘delta’ is defined as the difference between methylation levels of two adjacent CpGs in adjacent CpGs' pair.

The term “CpGs' pairs based methylation profile” as used herein means a set of methylation level differences, each associated with a particular pair of adjacent CpG sites defined by their genomic coordinates

The term “discriminative CpGs' pairs based methylation profile” as used herein means a selected set of methylation level differences, each associated with a particular selected pair of adjacent CpG sites defined by their genomic coordinates, the selected set of pairs being specific for biological sample and common between biological samples collected from the same sites and with the same procedures from one individual, as well as between biological samples collected from the same sites and with the same procedures from different individuals, if possible, at least to some extent, in both cases, irrespectively of individual's age, gender and other possible confounding factors.

The term “DNA modification/DNA treatment” refers at least to the conversion of an unmethylated cytosine to another nucleotide which will distinguish an unmethylated cytosine from a methylated cytosine. For example, an agent modifies unmethylated cytosine to uracil. Such an agent may be any agent conferring said conversion, wherein unmethylated cytosine is modified, but not methylated cytosine. For example, the agent for modifying unmethylated cytosine is sodium bisulfite. Sodium bisulfite (NaHSO3) reacts readily with the 5,6-double bond of cytosine, but only poorly with methylated cytosine. The cytosine reacts with the bisulfite ion, forming a reaction intermediate in the form of a sulfonated cytosine which is prone to deamination, eventually resulting in a sulfonated uracil. Uracil can subsequently be formed under alkaline conditions which removes the sulfonate group. The above exemplary type of reaction is a necessary step in the preparation of DNA in the procedures/methods for assessing methylation levels.

The term “epigenetic level measurement” comprises at least the term “methylation level measurements”. The term “methylation level measurements” refers to any method of methylation level measuring, for example such as “microarray” which is a popular measurement technology utilizing Infinium I and Infinium II assay chemistry technologies. Currently it is available in 4 price variants: 27K, 450K, EPIC and EPIC v2.0. These microarrays can measure methylation for (respectively) 27, 450, 850 or 935 thousand CpG in the genome. Another exemplary measuring method is “whole-genome bisulfite sequencing” (WGBS). This is an alternative method for assessing genome methylation levels utilizing next generation sequencing methods, and unlike microarray methods, it allows the assessment of methylation levels on a genome-wide scale. However, any quantitative methylation assessment method can be used.

“Amplification” according to the present invention is the process wherein a plurality of exact copies of one or more gene loci or gene portions (template) is synthesized. In one preferred embodiment of the present invention, amplification of a template comprises the process wherein a template is copied by a nucleic acid polymerase or polymerase homologue, for example a DNA polymerase or an RNA polymerase. For example, templates may be amplified using reverse transcription, the polymerase chain reaction (PCR), ligase chain reaction (LCR), in vivo amplification of cloned DNA, isothermal amplification techniques, and other similar procedures capable of generating a complementing nucleic acid sequence.

### Key Findings Relating to Co-Methylation and Non Co-Methylation Status in Pairs of Adjacent CpGs

It has been found that in humans, DNA methylation occurs almost exclusively at CpG dinucleotides which are non-randomly distributed in the genome with the regions of the higher-than-expected density of CpG sites referred to as CpG islands (CGI) (Gardiner-Garden M, Frommer M. CpG islands in vertebrate genomes). It is generally accepted that the methylation status of two consecutive CpG sites spaced less than 50 bp is correlated (Affinito O, Palumbo D, Fierro A, Cuomo M, De Riso G, Monticelli A, Miele G, Chiariotti L, Cocozza S. Nucleotide distance influences co-methylation between nearby CpG sites; Eckhardt F, Lewin J, Cortese R, Rakyan V K, Attwood J, Burger M, Burton J, Cox T V, Davies R, Down T A, Haefliger C, Horton R, Howe K, Jackson D K, Kunde J, Koenig C, Liddle J, Niblett D, Otto T, Pettett R, Seemann S, Thompson C, West T, Rogers J, Olek A, Berlin K, Beck S. DNA methylation profiling of human chromosomes 6, 20 and 22; Guo S, Diep D, Plongthongkum N, Fung H L, Zhang K, Zhang K. Identification of methylation haplotype blocks aids in deconvolution of heterogeneous tissue samples and tumor tissue-of-origin mapping from plasma DNA; Haerter J O, Lövkvist C, Dodd I B, Sneppen K. Collaboration between CpG sites is needed for stable somatic inheritance of DNA methylation states; Hu K, Ting A H, Li J. BSPAT: a fast online tool for DNA methylation co-occurrence pattern analysis based on high-throughput bisulfite sequencing data; Lovkvist C, Dodd I B, Sneppen K, Haerter J O. DNA methylation in human epigenomes depends on local topology of CpG sites). This phenomenon is referred to as co-methylation and methylated or non-methylated status of all CpG sites within CGI is considered to be essential for the regulatory function of those regions.

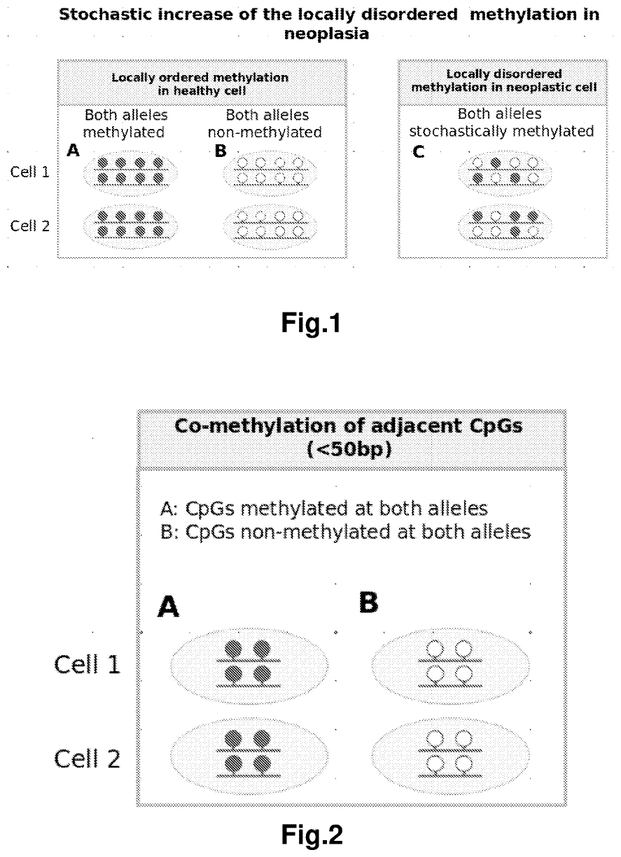

As it can be seen in FIG. 1 part A and FIG. 1 part B the status of methylation at single CpG site can be unmethylated or methylated. Taking into account that typically, nucleic acid in a single cell has two alleles in addition to the reproductive cells, different combination of status of methylation can be observed regarding mutual relation of the same CpG site at different alleles as well as across a certain region along one allele. For the purpose of clarity two cells have been depicted to show that one sample is a mixture of plurality cells what cannot be omitted during measurements of methylation level at particular CpG site.

The first type of methylation patterns is co-methylation patterns as presented on FIG. 1 part A and FIG. 1 part B. In such a case the status of methylation at each consecutive single CpG site both in a region and on two alleles is constant (all CpGs are methylated or all CpGs are unmethylated). The opposite case occurs when two consecutive CpG sites in a region or the same CpG site on two different alleles have different methylation status. This can be seen in FIG. 1 part C. Disruption of methylation at adjacent CpG sites was studded at single-cell resolution in neoplasia and is considered a feature of carcinogenesis, acting in similar way as stochastic accumulation of mutations. This phenomenon is referred to as “locally disordered methylation” or described as “stochastically disordered methylation in malignant cells” and is defined as a proportion of reads discordant for methylation within a specific locus (Landau D A, Clement K, Ziller M J, Boyle P, Fan J, Gu H, Stevenson K, Sougnez C, Wang L, Li S, Kotliar D, Zhang W, Ghandi M, Garraway L, Fernandes S M, Livak K J, Gabriel S, Gnirke A, Lander E S, Brown J R, Neuberg D, Kharchenko P V, Hacohen N, Getz G, Meissner A, Wu C J. Locally disordered methylation forms the basis of intratumor methylome variation in chronic lymphocytic leukemia, Klughammer J, Kiesel B, Roetzer T, Fortelny N, Nemc A, Nenning K H, Furtner J, Sheffield N C, Datlinger P, Peter N, Nowosielski M, Augustin M, Mischkulnig M, Ströbel T, Alpar D, Ergüner B, Senekowitsch M, Moser P, Freyschlag C F, Kerschbaumer J, Thomé C, Grams A E, Stockhammer G, Kitzwoegerer M, Oberndorfer S, Marhold F, Weis S, Trenkler J, Buchroithner J, Pichler J, Haybaeck J, Krassnig S, Mahdy Ali K, von Campe G, Payer F, Sherif C, Preiser J, Hauser T, Winkler P A, Kleindienst W, Würtz F, Brandner-Kokalj T, Stultschnig M, Schweiger S, Dieckmann K, Preusser M, Langs G, Baumann B, Knosp E, Widhalm G, Marosi C, Hainfellner J A, Woehrer A, Bock C. The DNA methylation landscape of glioblastoma disease progression shows extensive heterogeneity in time and space; Landan G, Cohen N M, Mukamel Z, Bar A, Molchadsky A, Brosh R, Horn-Saban S, Zalcenstein D A, Goldfinger N, Zundelevich A, Gal-Yam E N, Rotter V, Tanay A. Epigenetic polymorphism and the stochastic formation of differentially methylated regions in normal and cancerous tissues, Johnson K C, Anderson K J, Courtois E T, Gujar A D, Barthel F P, Varn F S, Luo D, Seignon M, Yi E, Kim H, Estecio M R H, Zhao D, Tang M, Navin N E, Maurya R, Ngan C Y, Verburg N, de Witt Hamer P C, Bulsara K, Samuels M L, Das S, Robson P, Verhaak R G W. Single-cell multimodal glioma analyses identify epigenetic regulators of cellular plasticity and environmental stress response; Gaiti F, Chaligne R, Gu H, Brand R M, Kothen-Hill S, Schulman R C, Grigorev K, Risso D, Kim K T, Pastore A, Huang KY, Alonso A, Sheridan C, Omans N D, Biederstedt E, Clement K, Wang L, Felsenfeld J A, Bhavsar E B, Aryee MJ, Allan J N, Furman R, Gnirke A, Wu C J, Meissner A, Landau D A. Epigenetic evolution and lineage histories of chronic lymphocytic leukaemia. In general, ‘locally disordered methylation’ is an opposition of ‘co-methylation’, thus can be also called a non co-methylation within a region. Locally disordered methylation is in general considered (similarly to genetic instability) to be random and enhance “the ability of cancer cells to search for superior evolutionary trajectories”. As mentioned, said phenomena was associated with tumor evolution (Gaiti F, Chaligne R, Gu H, Brand R M, Kothen-Hill S, Schulman R C, Grigorev K, Risso D, Kim K T, Pastore A, Huang K Y, Alonso A, Sheridan C, Omans N D, Biederstedt E, Clement K, Wang L, Felsenfeld J A, Bhavsar E B, Aryee M J, Allan J N, Furman R, Gnirke A, Wu C J, Meissner A, Landau D A. Epigenetic evolution and lineage histories of chronic lymphocytic leukaemia), shown to disturb local and distal gene expression (Klughammer J, Kiesel B, Roetzer T, Fortelny N, Nemc A, Nenning K H, Furtner J, Sheffield N C, Datlinger P, Peter N, Nowosielski M, Augustin M, Mischkulnig M, Ströbel T, Alpar D, Ergüner B, Senekowitsch M, Moser P, Freyschlag C F, Kerschbaumer J, Thomé C, Grams A E, Stockhammer G, Kitzwoegerer M, Oberndorfer S, Marhold F, Weis S, Trenkler J, Buchroithner J, Pichler J, Haybaeck J, Krassnig S, Mahdy Ali K, von Campe G, Payer F, Sherif C, Preiser J, Hauser T, Winkler P A, Kleindienst W, Würtz F, Brandner-Kokalj T, Stultschnig M, Schweiger S, Dieckmann K, Preusser M, Langs G, Baumann B, Knosp E, Widhalm G, Marosi C, Hainfellner J A, Woehrer A, Bock C. The DNA methylation landscape of glioblastoma disease progression shows extensive heterogeneity in time and space, Easwaran H, Tsai H C, Baylin S B. Cancer epigenetics: tumor heterogeneity, plasticity of stem-like states, and drug resistance) and most importantly was associated with adverse clinical outcome in different neoplasia e.g., CLL (Landau D A, Clement K, Ziller M J, Boyle P, Fan J, Gu H, Stevenson K, Sougnez C, Wang L, Li S, Kotliar D, Zhang W, Ghandi M, Garraway L, Fernandes S M, Livak K J, Gabriel S, Gnirke A, Lander E S, Brown J R, Neuberg D, Kharchenko P V, Hacohen N, Getz G, Meissner A, Wu C J. Locally disordered methylation forms the basis of intratumor methylome variation in chronic lymphocytic leukemia) or glioma (Johnson K C, Anderson K J, Courtois E T, Gujar A D, Barthel F P, Varn F S, Luo D, Seignon M, Yi E, Kim H, Estecio M R H, Zhao D, Tang M, Navin N E, Maurya R, Ngan C Y, Verburg N, de Witt Hamer P C, Bulsara K, Samuels M L, Das S, Robson P, Verhaak R G W. Single-cell multimodal glioma analyses identify epigenetic regulators of cellular plasticity and environmental stress response).

Based on said knowledge, the authors of the invention decided to analyze said phenomenon of co-methylation status (illustrated yet in FIG. 1 par A and B) and non co-methylation status (illustrated yet in FIG. 1 part C) across the smallest possible genomic region, namely a pair of adjacent CpG sites (as can be seen in FIG. 2 and FIG. 3). The data that was used in this experiment reflect methylation ratio of all cells in the biological sample. FIG. 2 present two types of co-methylation in pairs of adjacent CpG sites in individual cells composing a biological sample, that result in the identical measurement of the co-methylation with the technology used by the authors of the invention. FIG. 3 present three types of non co-methylation in individual cells composing a biological sample, that result in the identical measurement of the non co-methylation (also called discordant methylation) with the technology used by the authors of the invention.

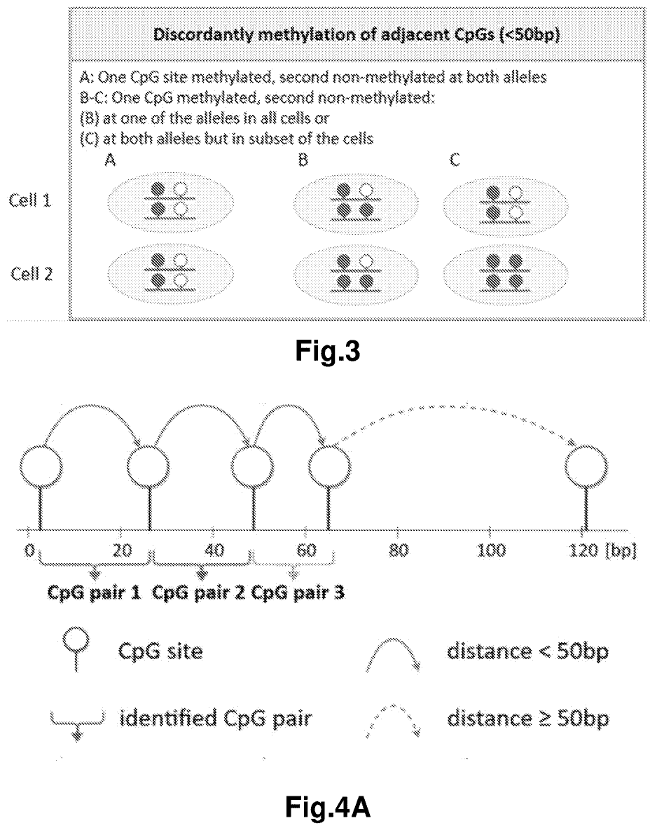

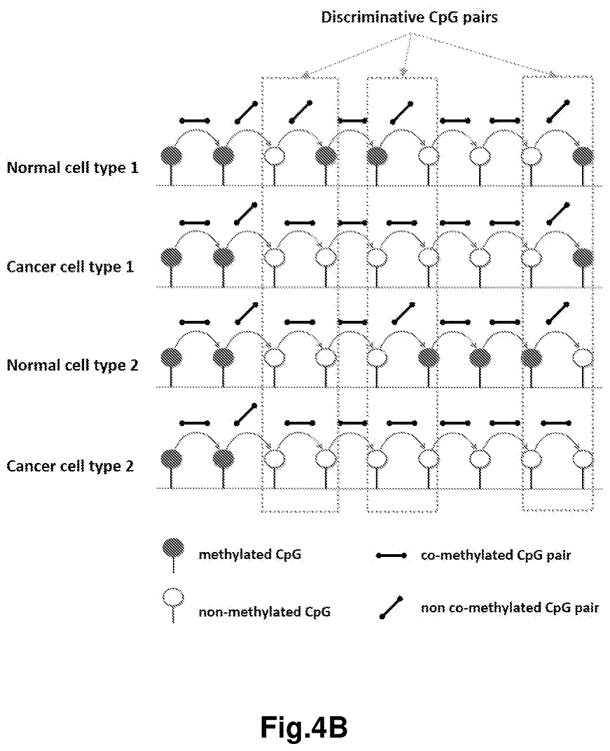

To be able to study mutual relation of the methylation level within such small regions, first said small regions' genomic coordinates have to be determined. FIG. 4A schematically shows the identification procedure of all pairs of CpG sites spaced less than a certain arbitrary threshold value. As it can be seen, adjacent CpG sites are linked into pairs. It should be noted that the second CpG site in one pair is also the first site in the second pair. Optionally, a set of CpG pairs can be a set of disjoint pairs. The distance threshold value can be set to take into account the correlation of methylation states observed in the state of the art. Said correlation value is the biggest for CpG sites located within a distance of less than 50 bp.

When analyzing samples with different types of tissues and cells across the human population, surprisingly the authors of the invention found that the non co-methylation phenomenon in the same pairs of adjacent CpG sites is repeatable within a group of samples relating to the same type of cell. In parallel, studies have shown that also co-methylation phenomena in the same pairs of adjacent CpG sites is repeatable within a group of samples relating to the same type of cell.

As a result of further studies, the authors of the invention have surprisingly found that detection of the difference in methylation status in a pair (co-methylation vs non co-methylation or non co-methylation vs co-methylation) between two different types of samples provides one of the biomarkers indicative of the origin and type of the cell or tissue contained in said samples. Here the term ‘biomarker should be understood as a pair of co-methylated or non co-methylated CpG sites with different methylation status between compared biological samples. Thus, a set of selected discriminative pairs of CpG sites (pairs of epigenetically modified bases in general) is the methylation signature of a certain type of tissue or cell according to the invention. The general concept is presented in FIG. 4B

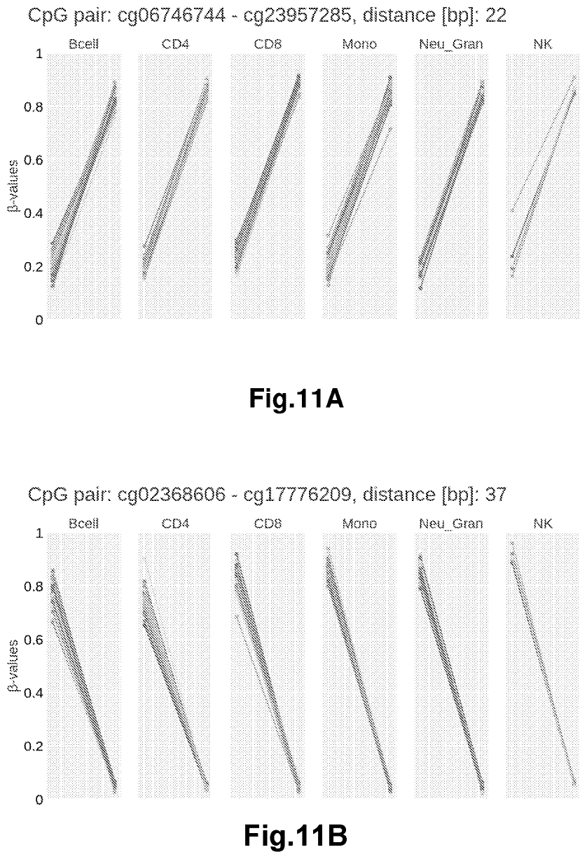

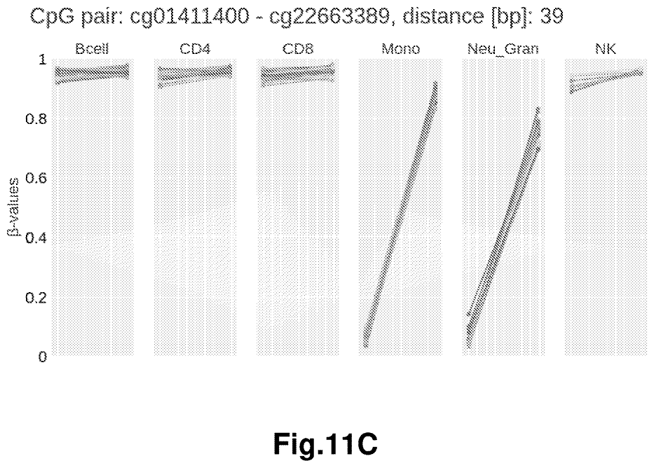

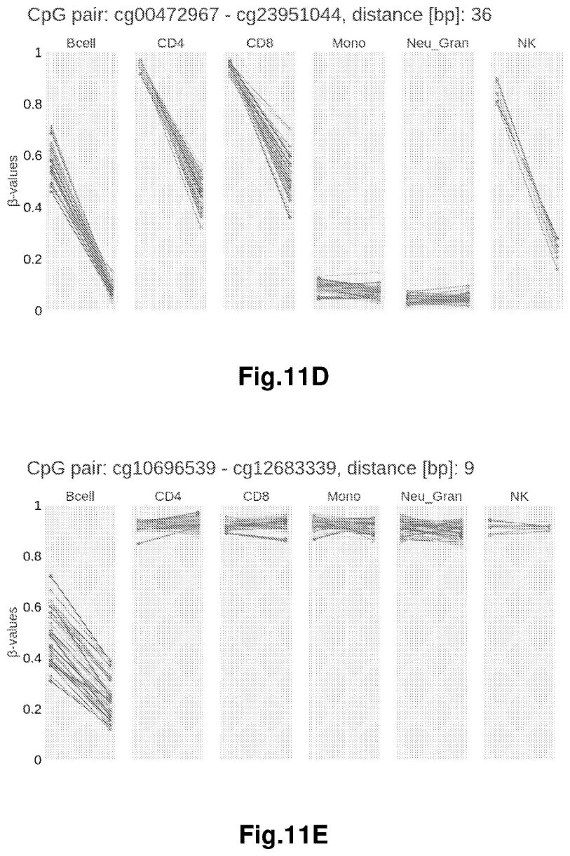

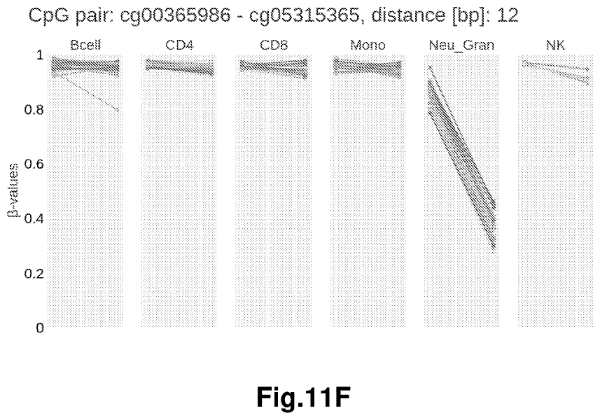

The authors of the invention proposed a new and inventive way of encoding the status of mutual relation of the methylation level for two CpG sites in a pair in a compressed and interpretable form, namely as one single numerical value, although the status in a pair can be easily interpreted also with the use of a typical graph presentation with the use of the raw data (see FIG. 11A-F). Said one single numerical value can be a result of any mathematical conversion of two methylation level values that is interpretable for that purpose. Advantageously, calculation of the difference between said two methylation level in a pair is an example of a mathematical conversion useful for said interpretation purpose.

Examples from Original Studies Relating to Co-Methylation and Non Co-Methylation Status in Pairs of Adjacent CpGs

In background studies, raw data (methylation level frames) relating to the methylation level of an individual CpG site in a cell population has been used. In particular, 136 EPIC microarray methylation profiles of the whole blood samples were used from four independent studies: GSE123914 (this data set included arrays for 35 individual blood samples, 34 with DNA methylation measurements at two time-points approximately one year apart) (Zaimi I, Pei D, Koestler D C, Marsit C J, De Vivo I, Tworoger S S, Shields A E, Kelsey K T, Michaud D S. Variation in DNA methylation of human blood over a 1-year period using the Illumina MethylationEPIC array), GSE153211 (n=8 arrays) (Cubellis M V, Pignata L, Verma A, Sparago A, Del Prete R, Monticelli M, Calzari L, Antona V, Melis D, Tenconi R, Russo S, Cerrato F, Riccio A. Loss-of-function maternal-effect mutations of PADI6 are associated with familial and sporadic Beckwith-Wiedemann syndrome with multi-locus imprinting disturbance), GSE112618 (n=6 arrays) (Salas L A, Koestler D C, Butler R A, Hansen H M, Wiencke J K, Kelsey K T, Christensen B C. An optimized library for reference-based deconvolution of whole-blood biospecimens assayed using the Illumina HumanMethylationEPIC BeadArray), GSE166844 (n=29 arrays; from 29 individuals, with 14 pairs being monozygotic twins) (Hannon E, Mansell G, Walker E, Nabais M F, Burrage J, Kepa A, Best-Lane J, Rose A, Heck S, Moffitt T E, Caspi A, Arseneault L, Mill J. Assessing the co-variability of DNA methylation across peripheral cells and tissues: Implications for the interpretation of findings in epigenetic epidemiology) and 24 validation blood samples from healthy individuals (obtained for validation purposes).

The analysis of methylation patterns resulting from pairs of CpGs was performed using EPIC microarrays obtained for purified white blood cell fractions, including: GSE110554 (neutrophils (n=6), monocytes (n=6), B cells (n=6), CD4+ T cells (n=7, six individual arrays and one technical replicate), CD8+ T cells (n=6), NK cells (n=6)) (Salas L A, Koestler D C, Butler R A, Hansen H M, Wiencke J K, Kelsey K T, Christensen B C. An optimized library for reference-based deconvolution of whole-blood biospecimens assayed using the Illumina HumanMethylationEPIC BeadArray), and GSE166844 (granulocytes (n=29), monocytes (n=28), B cells (n=28), CD4+ T cells (n=28), CD8+ T cells (n=28) (Hannon E, Mansell G, Walker E, Nabais M F, Burrage J, Kepa A, Best-Lane J, Rose A, Heck S, Moffitt T E, Caspi A, Arseneault L, Mill J. Assessing the co-variability of DNA methylation across peripheral cells and tissues: Implications for the interpretation of findings in epigenetic epidemiology).

The methylation level frames (methylation profiles) for subpopulations of B cells were obtained for cells sorted from blood of four healthy donors (protocol approved by the Regional Ethical Committee of Southern Denmark (Project-ID: S-20160069) and the Blood Bank at Odense University Hospital (Project nr: DP049), using MethylationEPIC Beadchip (Illumina) according to manufacturer protocol.

Changes in methylation patterns caused by the neoplastic transformation were investigated using EPIC methylation profiles of blood samples from patients with acute myeloid leukemia from dataset GSE124413 (n=458), with chronic lymphocytic leukemia (n=114) previously described in (Hussmann D, Starnawska A, Kristensen L, Daugaard I, Thomsen A, Kjeldsen T E, Hansen C S, Bybjerg-Grauholm J, Johansen K D, Ludvigsen M, Kristensen T, Larsen T S, Møller M B, Nyvold C G, Hansen L L, Wojdacz T K. IGHV-associated methylation signatures more accurately predict clinical outcomes of chronic lymphocytic leukemia patients than IGHV mutation load). The non co-methylation and co-methylation in healthy tissues was assessed using: GSE124413 (n=41) for bone morrow, GSE142141 (n=47) for skeletal muscle, GSE100850 (n=5) for breast tissue, and GSE132804 (n=206) for colon tissue.

Raw Illumina MethylationEPIC array data (.idat) were processed, QC (Quality Control) checked, and normalized with Beta Mixture Quantile (BMIQ) method (Teschendorff A E, Marabita F, Lechner M, Bartlett T, Tegner J, Gomez-Cabrero D, Beck S. A beta-mixture quantile normalization method for correcting probe design bias in Illumina Infinium 450 k DNA methylation data) in ChAMP Package® (Tian Y, Morris T J, Webster A P, Yang Z, Beck S, Feber A, Teschendorff A E. ChAMP: updated methylation analysis pipeline for Illumina BeadChips, Morris T J, Butcher L M, Feber A, Teschendorff A E, Chakravarthy A R, Wojdacz T K, Beck S. ChAMP: 450k Chip Analysis Methylation Pipeline). All the genomic variations, such as SNPs (single-nucleotide polymorphism) shown to may influence methylation analysis results were filtered out using basic implementation of ChAMP pipeline (Zhou W, Laird P W, Shen H. Comprehensive characterization, annotation and innovative use of Infinium DNA methylation BeadChip probes).

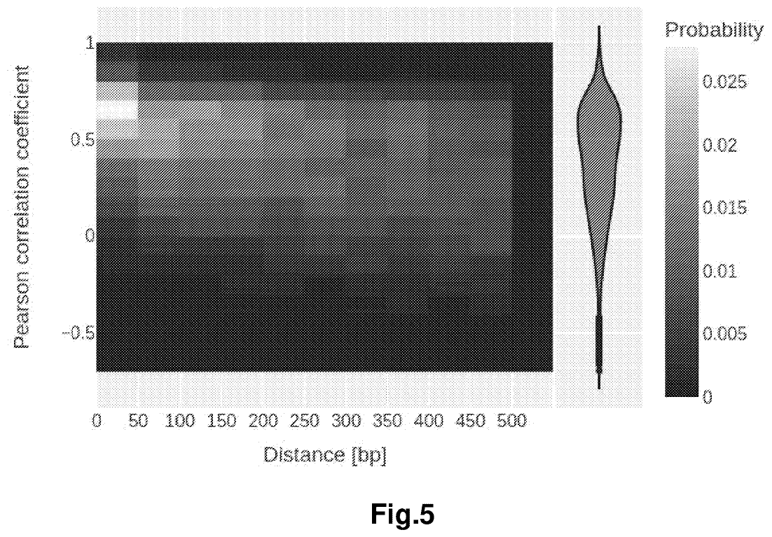

The correlation of single CpG site methylation levels derived from raw data used in the studies was checked and the results are presented in FIG. 5. The heat map shows correlation between the methylation level and the distance of 10 000 randomly selected CpG sites from 136 EPIC arrays for healthy blood. The x axis shows the distance between CpG sites and y axis shows Pearson correlation coefficient. The results of said check confirmed previous observations that there is a strong correlation between methylation status of the adjacent CpG sites.

As analysis was based on relative differences of methylation levels—referenced also as delta methylation level within each microarray and subsequent comparison of that difference between individual microarray the data set containing microarrays obtained for whole blood DNA for blood cell type comparison were not corrected.

The use of that correction could only improve the results of the analysis. However, the EpiDISH package using “centDHSbloodDMC.m” reference methylation profiles and CIBERSORT (CBS) method was used to estimate the proportions of white blood cell types in the whole blood samples (Teschendorff A E, Breeze C E, Zheng S C, Beck S. A comparison of reference-based algorithms for correcting cell-type heterogeneity in Epigenome-Wide Association Studies, Reinius L E, Acevedo N, Joerink M, Pershagen G, Dahlén S E, Greco D, SoderhAll C, Scheynius A, Kere J. Differential DNA methylation in purified human blood cells: implications for cell lineage and studies on disease susceptibility). This analysis showed that blood samples did differ in the cell type composition.

All hierarchical clustering analyses were performed using Ward's method and Euclidean distance. The variability of delta beta-values was calculated as the mean standard deviation in methylation level at each site for each sample type and results were displayed as box-plots with an estimation of probability density function using kernel density estimator.

The procedure of linking CpG sites into pairs was applied to EPIC microarray data and 126 743 CpG pairs sites spaced less than 50 bp were identified. Next, the difference in methylation levels (delta methylation level) between CpG dinucleotides within each of the identified dinucleotide pairs using 136 EPIC arrays obtained for healthy blood samples from 5 independent studies was calculated.

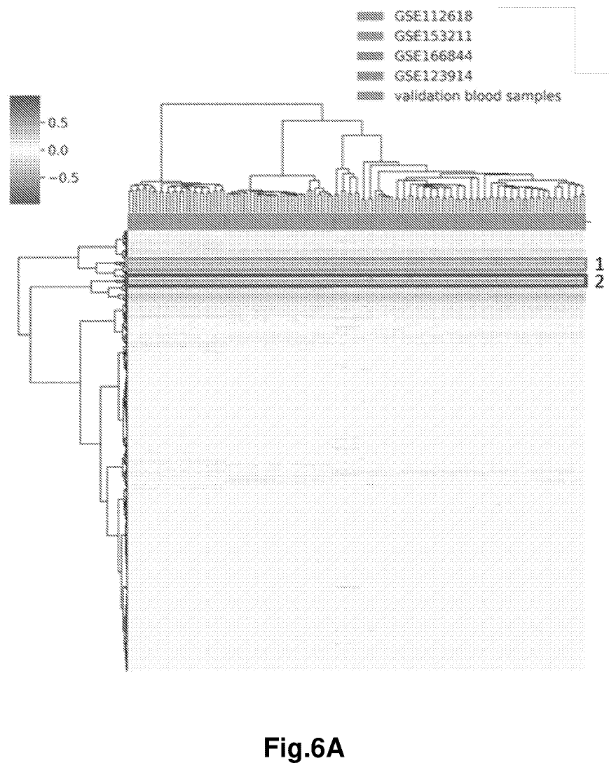

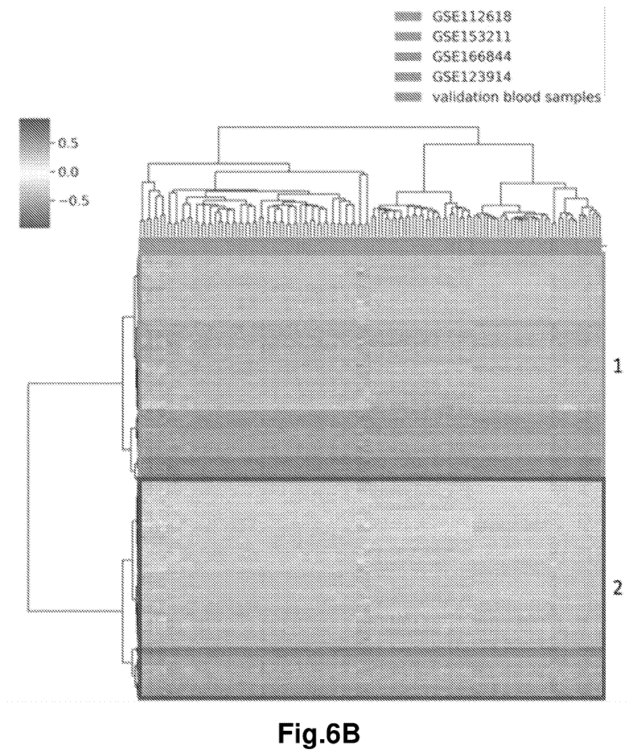

Methylation status of the majority of adjacent CpG sites was identical (delta methylation level close to 0). The result of this experiment can be observed in FIG. 6A, wherein white color indicates no difference in delta methylation level within a CpG pair. However interestingly and unexpectedly two sets of the CpG pairs with obviously non co-methylation were also identified (FIG. 6A, green Box 1 and red Box 2).

Despite a minor study-specific batch effect that makes microarrays from different experiments to form separate clusters in unsupervised clustering analyses (better seen in FIG. 6B which represents only selected subset of pairs with non co-methylation state), the difference in methylation level between those nucleotides was remarkably stable for a specific type of sample between individual methylation profiles from four independent data sets.

The heatmap from unsupervised clustering based on the methylation level value difference between CpG sites of the subset of CpG pairs which is shown in FIG. 6B clearly confirms at least the remarkable stability of identified non co-methylation patterns in healthy blood samples. Both, relative increase (FIGS. 6A and 6B, Box 1) and relative decrease (FIGS. 6A and 6B, Box 2) of methylation level between nucleotides were observed. However, change in the direction is only attributed to the technical aspects of the data processing (delta beta-value, i.e. methylation level value difference calculation direction).

Then, the methylation level difference between nucleotides in each pair was quantified (namely delta methylation level value was calculated) and further analysis focused first only on the pairs displaying more than about 0.3 delta methylation level-value, (namely displaying non co-methylation status).

This cut off was set with the rationale that observed difference in beta-value should reflect the change of methylation status within one of the two alleles in a cell and considering limitations of the bead array technology the beta-value difference of more than 0.3 is likely to reflect that change. This analysis identified 2470 CpG nucleotide pairs in EPIC profiles obtained for DNA extracted from healthy whole blood samples with a mean delta methylation level between those adjacent nucleotides of 0.407 (95% CI: 0.40-0.41) and the average distance between CpG sites in the pair of 28 bp.

In additional background studies another technology, namely Sanger sequencing of bisulfite pretreated DNA, was used to confirm two aspects of the studies: first that non co-methylation status of CpG sites in selected pairs of CpG sites in 24 peripheral blood samples (obtained for validation purposes) can be observable using also a qualitative measurements method; second, that the qualitative measurement methods gives back exactly the same results for the same selected CpG pairs as a quantitative measurement method. Sequencing results were analyzed using Chromas 2.6.6 program.

Validation of methylation status of selected non co-methylated CpG pairs, using Sanger sequencing of bisulfite pretreated DNA, confirmed differences in methylation status between adjacent CpG sites in analyzed pairs.

In principle bisulfite pretreatment of DNA converts all non methylated cytosine (C) to uracil (U), which is subsequently converted to thymine (T) during amplification while methylated cytosines remain unchanged. Therefore, in selected non co-methylated CpG pairs, we expected to observe the conversion of one cytosine within CpG pair into thymine, with simultaneous lack of conversion of the adjacent cytosine.

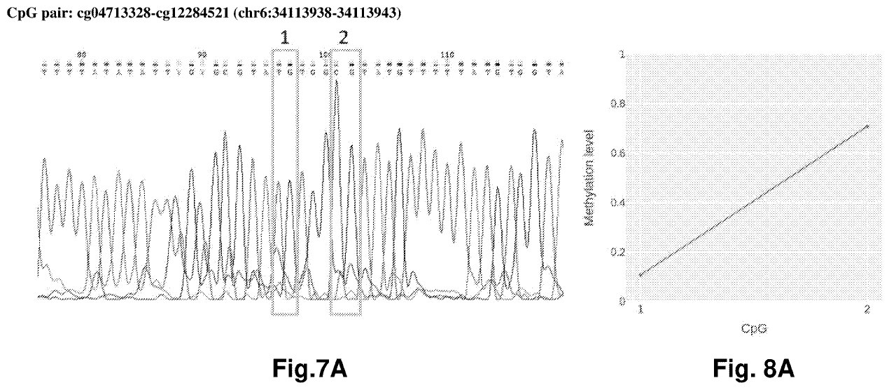

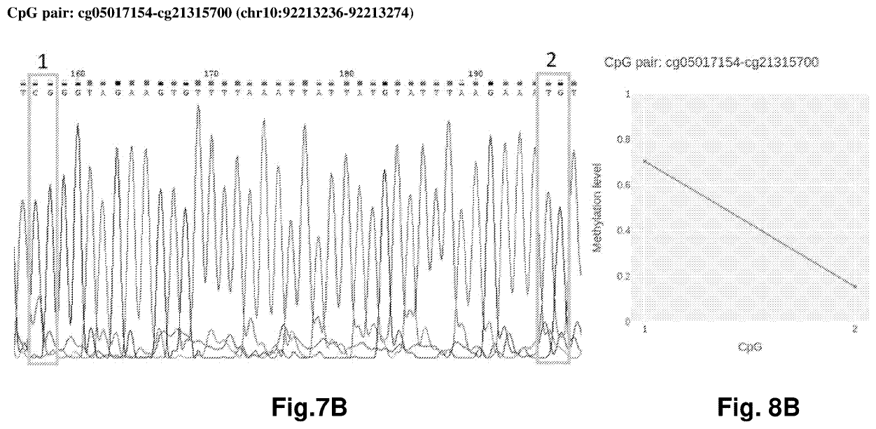

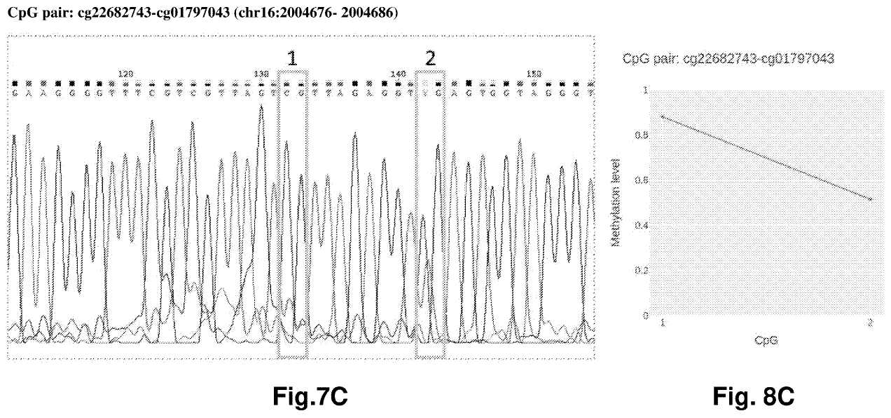

The examples of three Sanger sequencing chromatograms, and corresponding raw data from quantitative measurements of non co-methylated CpG pairs are presented in FIG. 7a-c and FIG. 8a-c, respectively. The non co-methylation status of CpG sites within CpG pair is confirmed, when one of the cytosines is non methylated and thus displayed in the chromatogram as nucleotide T at a given genomic position on both alleles (Box 1, FIG. 7a; Box 2, FIG. 7b) or double sequence representing nucleotides C and Tat a given genomic position on T (shown in the chromatogram as “Y”; Box 2, FIG. 7C), while the second adjacent CpG is methylated and thus displayed as nucleotide C in the chromatogram (Box 2, FIG. 7a; Box 1, FIG. 7b; Box 1, FIG. 7c).

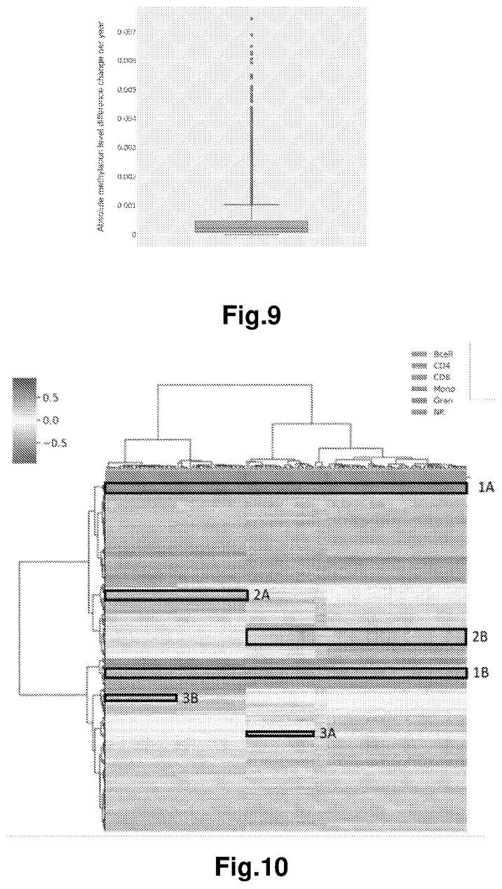

As methylation patterns have been shown in the state of the art to change with age, the authors of the invention assessed whether the methylation level difference between two consecutive CpG sites also changes with age. This phenomenon has been checked for all CpG pairs, including ones that basically presented co-methylated status, using data acquired from 136 EPIC microarray methylation profiles of the whole blood samples (from four independent studies: GSE123914 (this data set included arrays for 35 individual blood samples, 34 with DNA methylation measurements at two time-points approximately one year apart) (Zaimi I, Pei D, Koestler D C, Marsit C J, De Vivo I, Tworoger S S, Shields A E, Kelsey K T, Michaud D S. Variation in DNA methylation of human blood over a 1-year period using the Illumina MethylationEPIC array), GSE153211 (n=8 arrays) (Cubellis M V, Pignata L, Verma A, Sparago A, Del Prete R, Monticelli M, Calzari L, Antona V, Melis D, Tenconi R, Russo S, Cerrato F, Riccio A. Loss-of-function maternal-effect mutations of PADI6 are associated with familial and sporadic Beckwith-Wiedemann syndrome with multi-locus imprinting disturbance), GSE112618 (n=6 arrays) (Salas L A, Koestler D C, Butler R A, Hansen H M, Wiencke J K, Kelsey K T, Christensen B C. An optimized library for reference-based deconvolution of whole-blood biospecimens assayed using the Illumina HumanMethylationEPIC BeadArray), GSE166844 (n=29 arrays; from 29 individuals, with 14 pairs being monozygotic twins) (Hannon E, Mansell G, Walker E, Nabais M F, Burrage J, Kepa A, Best-Lane J, Rose A, Heck S, Moffitt T E, Caspi A, Arseneault L, Mill J. Assessing the co-variability of DNA methylation across peripheral cells and tissues: Implications for the interpretation of findings in epigenetic epidemiology) and 24 blood samples from healthy individuals (obtained for validation purposes)). It was observed that 90% of all tested CpG pairs (n=96 766) display remarkably stable methylation level differences between CpGs in CpG pairs (absolute methylation level difference change per year<0.001). However, the remaining 10% of pairs display association with age (absolute methylation level difference change in CpG pair per year>0.001) which may be useful for the age prediction purposes (FIG. 9).

To asses and verify stability and cell specificity of delta beta values calculated for CpG pairs of sorted blood cells (using FACS method) including: granulocytes, monocytes, B cells, CD4+ T cells, CD8+ T cells, cells NK were used to elaborate whether at least non co-methylation pattern differs between blood cells types. This analysis found 2794 non co-methylated CpG pairs in granulocytes, 2506 in monocytes, 2522 in B cells, 2751 in CD4+ T cells, and 2666 in CD8+ T-cells, 2609 in NK cells.

The unsupervised clustering analyses of delta beta-values at identified CpG pairs showed that some of them displayed identical levels of the non co-methylation in all analyzed cell types (FIG. 10, Box 1A and 1B). Other were non co-methylated in cells from one hematopoietic lineage e.g., myeloid (FIG. 10, Box 2A) or lymphoid (FIG. 10, Box 2B) and displayed co-methylation in lymphoid and myeloid lineages, respectively. There were also subsets of CpG sites that displayed non co-methylation in only one type of cells within lymphoid or myeloid lineage, e. g. B-cells (FIG. 10, Box 3A) or granulocytes (FIG. 10, Box 3B).

Since the delta methylation level-values represent only a relative information, namely methylation level difference for two CpG sites, to analyze dynamics of the methylation level differences at non co-methylated CpG sites between blood cell types, beta-values between CpG sites within non co-methylated CpG pairs were compared for said sorted blood cell samples of the same type by plotting raw data on methylation levels in two selected CpG pairs vs their distance in bp (see FIG. 11a-11f).

Without wishing to be bound to any particular theory, applicant believes that compressed epigenetic modification information for a CpG pair according to the invention allows to interpret each discriminative pair in the signature related to a specific cell or tissue for the change of the methylation status on one or two alleles within a pair. FIG. 11a-11b, illustrate CpG pairs with the same pattern of non co-methylation in all cell types tested. Those levels of methylation difference most likely indicate non co-methylation present at both allele.

Secondly, CpG sites can have different methylated pattern in hematopoietic cell lineage specific manner. FIG. 11c illustrates CpG sites within a pair which is non co-methylated with 100% methylation level difference in myeloid lineage cells (one CpG site methylated and one CpG site unmethylated), while said pair of CpG sites is co-methylated in lymphoid cells (both CpG sites methylated).

Opposite to non co-methylation pattern of CpG pair shown in FIG. 11c, FIG. 11d shows CpG sites within another pair, said pair being also non co-methylated in lymphoid cells but with about 50% methylation level difference (methylated only at one allele), while in myeloid cell-linage said another pair of CpG sites is co-methylated (with both CpG sites being unmethylated).

Lastly, the present data analysis showed that the methylation pattern represented by delta beta-values for CpG pairs can dynamically change between cell types. The CpG sites shown in FIG. 11e-11f, are non co-methylated only in one type of cells within lymphoid or myeloid lineage. Specifically, FIG. 11e shows CpG sites non co-methylated in B cells with methylation change suggesting non co-methylation of one allele, while in other types of cells the CpG sites are co-methylated Similarly, CpGs shown in FIG. 11f are non co-methylated in granulocytes, and co-methylated in all other types of cells, namely in monocytes, as well as in all lymphoid cell types.

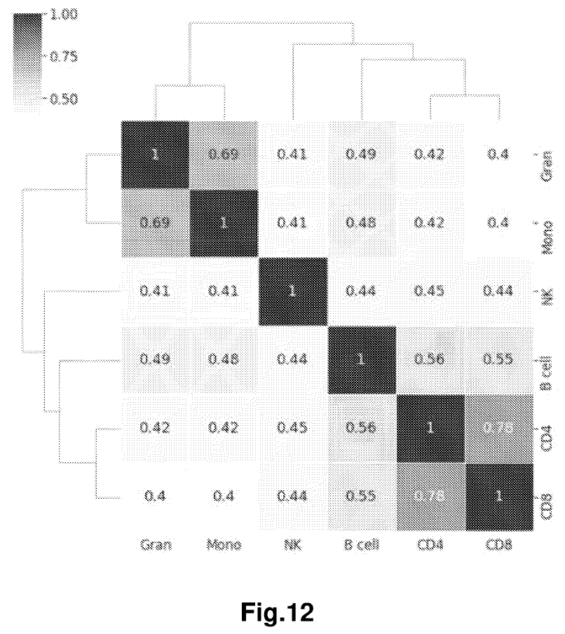

Next, to investigate to what extent at least non co-methylated CpG pairs differ between blood cell types, the number of overlapping CpG pairs identified for each cell type were compared and calculated the Jaccard similarity index comparing CpG pairs that are common between each two types of WBC (FIG. 12). This analysis showed that the most CpG pairs with the similar non co-methylation status was between CD4+ T cells and CD8+ T cells (at the level of 0.78), as well as granulocytes and monocytes (0.69). Whereas, the overlap of this type of CpG pairs was markedly smaller for cells from different hematopoietic lineages e.g., for granulocytes and CD4+ T cells or monocytes and CD8+ T the overlap was at the level of 0.42 and 0.40, respectively. This again indicates that at least non co-methylation patterns are hematopoietic lineage specific.

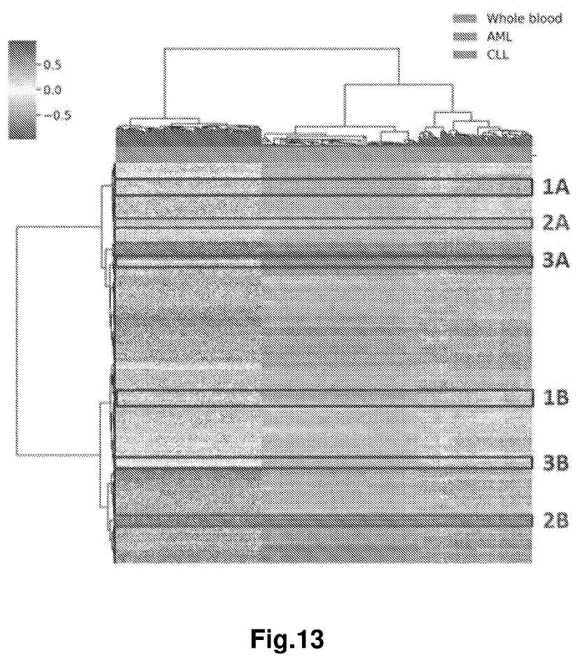

As known from the prior art, locally disordered methylation of the adjacent CpG sites has been reported in cancer cells and is generally considered “to arise from stochastically disordered methylation in malignant cells”, but surprisingly studies performed by the authors of the invention contradict with that principle. To analyze the changes of methylation patterns between healthy blood and malignant cells at least the methylation status of CpG sites that were stably non co-methylated in all of the WBC fractions was compared between healthy blood samples, and two hematological malignancies: acute myeloid leukemia (AML, n=458) and chronic lymphocytic leukemia (CLL, n=114) (FIG. 13). This analysis was performed using only said stably non co-methylated subset of CpG sites to reduce interexperimental variability that CpG sites with different methylation status in different blood cells would cause.

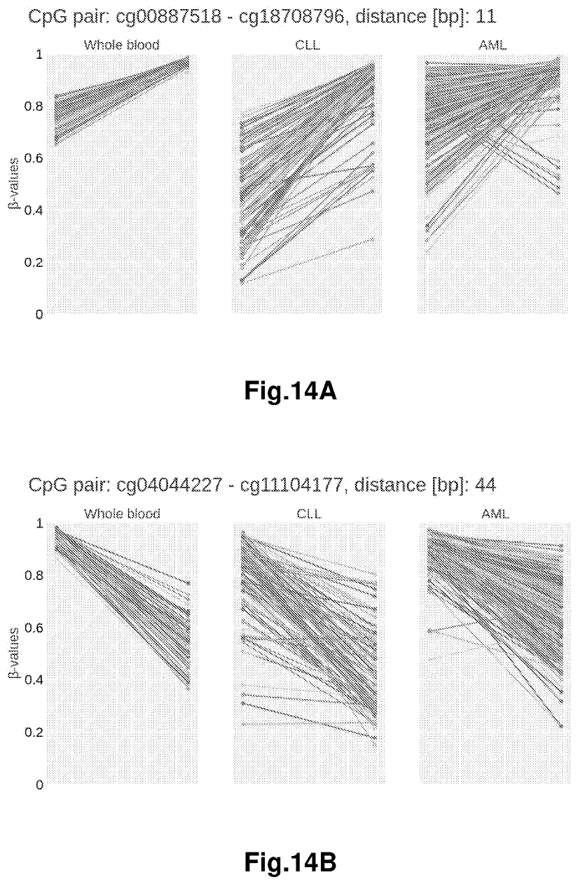

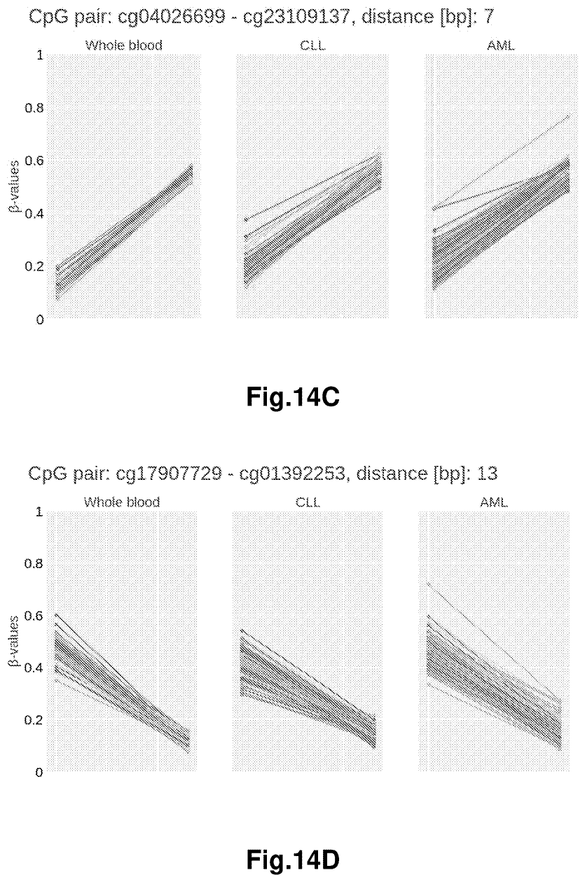

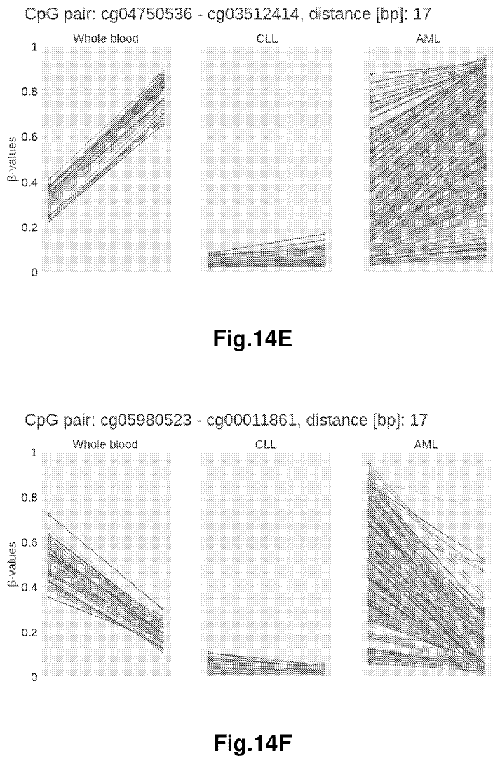

Overall, unsupervised hierarchical clustering based on this subset of non co-methylated CpG sites showed remarkable heterogeneity of methylation changes in both CLL and AML (see FIG. 13). The majority of observed methylation changes in neoplastic cells involved both CpG sites of the non co-methylated CpG pair and observed changes in individual neoplastic samples appeared to be random (see heatmap in FIG. 13, Boxes 1A-1B and corresponding line plots in FIG. 14A-14B). Nevertheless, CpG sites that maintained non co-methylation pattern in both neoplastic cell types were identified ((see heatmap in FIG. 13, Boxes 2A-2B, and corresponding line plots FIG. 14C-14D), as well as CpG sites that lost non co-methylation and become co-methylated in CLL but in AML appeared to display randomly disturbed methylation pattern ((see heatmap in FIG. 13, Box 3A and corresponding line plots FIG. 14E).

Similarly, there were CpG sites that lost non co-methylation in CLL but remained the pattern observed in healthy blood in majority of the AML samples (see heatmap in FIG. 13, Box 3B, and corresponding line plots in FIG. 14F)

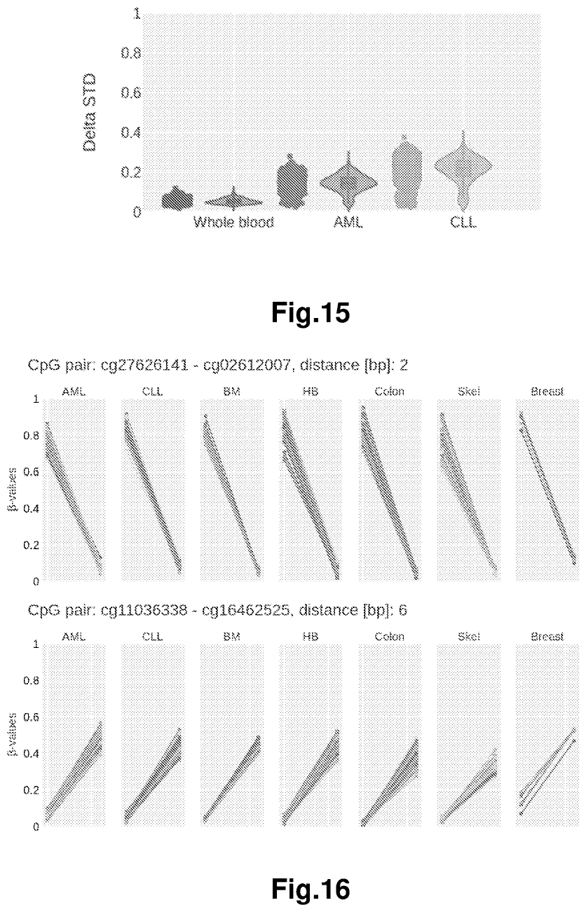

With the indication that non co-methylation changes are neoplasia specific, the variance of the beta-values at non co-methylated CpG sites between CLL, AML and healthy blood were compared. The violin plots in FIG. 15 show that the standard deviation (STD) of the delta beta-values in both CLL (0.206 (IQR: 0.080)), and AML (0.142 (IQR: 0.052)) is significantly increased as opposed to the rather low STD in healthy blood (0.054 (IQR: 0.022, p<0.001, Kruskal-Wallis test). Moreover, the average level of the variance is statistically significantly different (p<0.001 Kruskal-Wallis test) between the two diseases.

Despite observed general trend for non co-methylation to stochastically change during neoplastic transformation, the heatmap in FIG. 13 also indicates that the methylation of at least a subset of the non co-methylated CpG sites is the same in malignant and healthy cells. To identify those CpG sites, it was attempted to find non co-methylated CpG pairs in CLL and AML data and the authors of the invention found 182 and 118 CpG pairs non co-methylated in CLL and AML, respectively.

Interestingly, 64 of non co-methylated CpG pairs were common between these two malignancies. The methylation levels at those CpG sites between both malignancies and five healthy tissues were compared and it was found that the methylation levels at this subset of CpG sites does not change between malignant and healthy tissues (example line plots for two CpG pairs shown in FIG. 16). This indicates that a subset of non co-methylated CpG sites is protected from change even during neoplastic transformation.

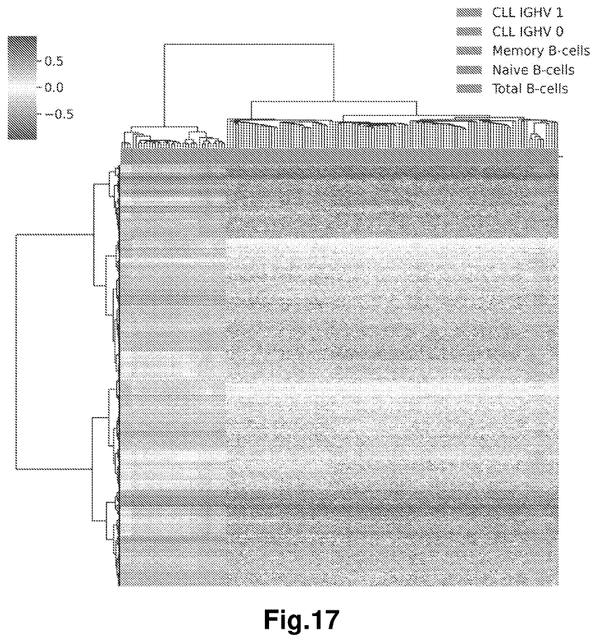

Differences in methylation level has been shown previously to take significant part in B cell maturation and it has been recently shown by the authors of the invention that IGHV mutation load associated methylation changes predict clinical outcomes of CLL patients more accurately than IGHV mutation load (24). As it was observed, there were different non co-methylation patterns in both naive and mature subtypes of B cell, and the clinical outcomes of CLL depend on the type of cells that the neoplastic transformation originates from, we were interested to see whether the methylation in IGHV unmutated CLL (CLL_IGHV_0) and IGHV mutated CLL (CLL_IGHV_1) change independently. The unsupervised hierarchical clustering of B cells subpopulations and CLL samples based on delta beta-values of CpG pairs identified in naive and memory B cells grouped naive B cells with CLL_IGHV_0 samples and memory B cells with CLL_IGHV_1 samples (see FIG. 17).

Although a striking difference in methylation pattern between CLL samples in each of two clusters in the heatmap was not observed, the fact that patients grouped into two main clusters corresponding to IGHV status, indicates that non co-methylation profiles of naive and memory B-cells undergo independent to at least some extend changes during malignant transformation of these two

types of CLL (FIG. 17).

The Time-To-Treatment (TTT) data were available for patients in this cohort and to elaborate whether stratification of the patients based on non co-methylation patterns improves prediction of clinical outcomes Kaplan-Meier analysis was performed based on patient stratification according to IGHV status and non co-methylation patterns. It was seen that classification based on non co-methylation could predict TTT, but the authors of the invention did not observe significant difference of the prediction power between these two classifications.

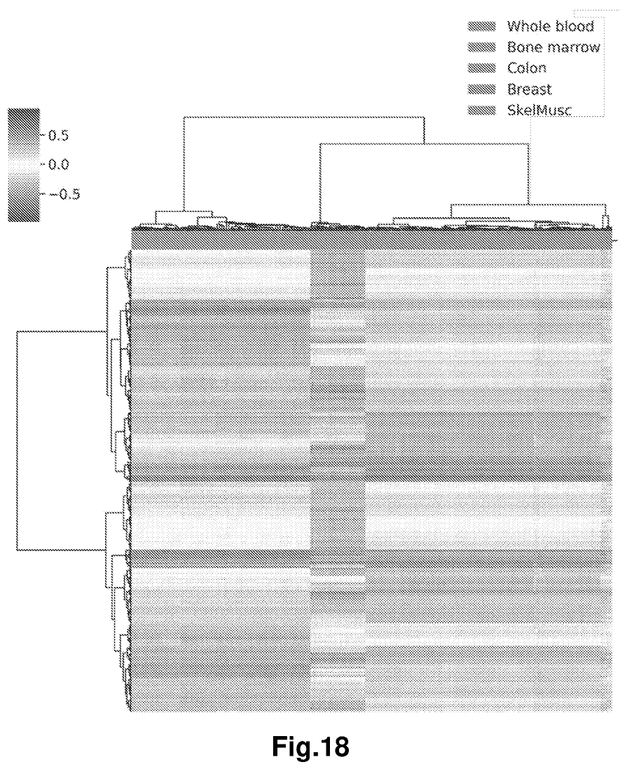

Next, also at least non co-methylation patterns in other healthy tissues have been studied. Analyses of blood cell types provided above indicated that methylation patterns are cell type specific. To extent this finding to other tissue types the authors of the invention analyzed non co-methylation patterns in: bone marrow (n=41), skeletal muscle (n=47), colon (n=206), and breast tissue (n=5). The analysis identified in bone marrow 1813, skeletal muscle 3266, colon 2084, and breast tissue 2178 non co-methylated CpG pairs. The unsupervised clustering analyses based on identified CpG sites showed without doubt that each tissue has highly stable and specific pattern of non co-methylation but even in developmentally distant tissues there are loci with the same status of non co-methylation (FIG. 18).

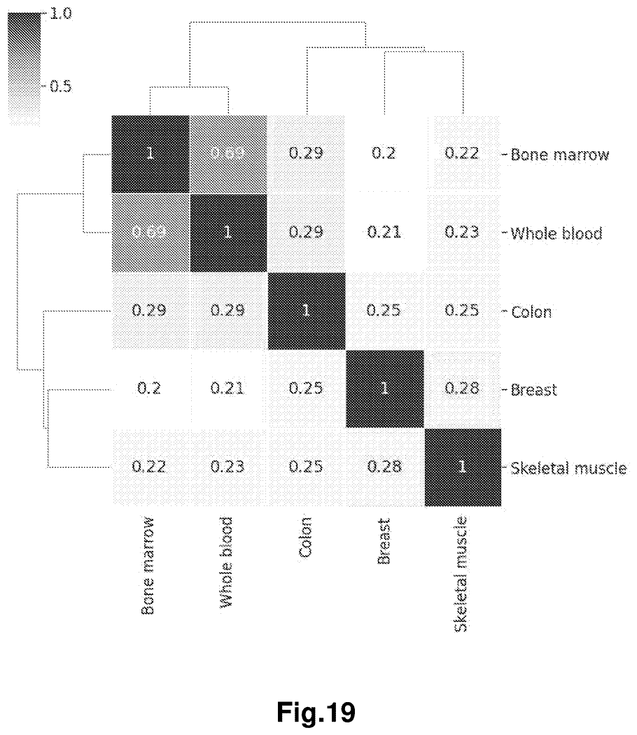

To analyze to what extent non co-methylated CpG pairs differ between different types of healthy tissues, the number of overlapping CpG pairs identified for each tissue type were compared and calculated the Jaccard similarity index comparing sets of CpGs between each two types of tissues (FIG. 19). This analysis showed that developmentally close tissues, such as blood and bone marrow, have the most similar non co-methylation patterns. This data were also processed to identify non co-methylated CpG pairs that display the same methylation levels in all analysis tissues and found that 328 of non co-methylated CpG sites with identical methylation levels across all tissues (not shown).

Finally in order to check an industrial application of the background studies at least non co-methylation patterns-based typing of healthy and malignant cells has been performed. The authors of the invention were able to identify a selected subset of non co-methylated CpG pairs with stable methylation difference in each CLL and AML, which indicates that unexpectedly not only healthy tissues but also neoplastic cells appear to have specific patterns of non co-methylation. It should be noted that all studies discussed above have been repeated also for mixture of pairs having non co-methylation and co-methylation status. Said further results allowed to draw exactly the same conclusions as presented.

### Overall Process of Industrial Application

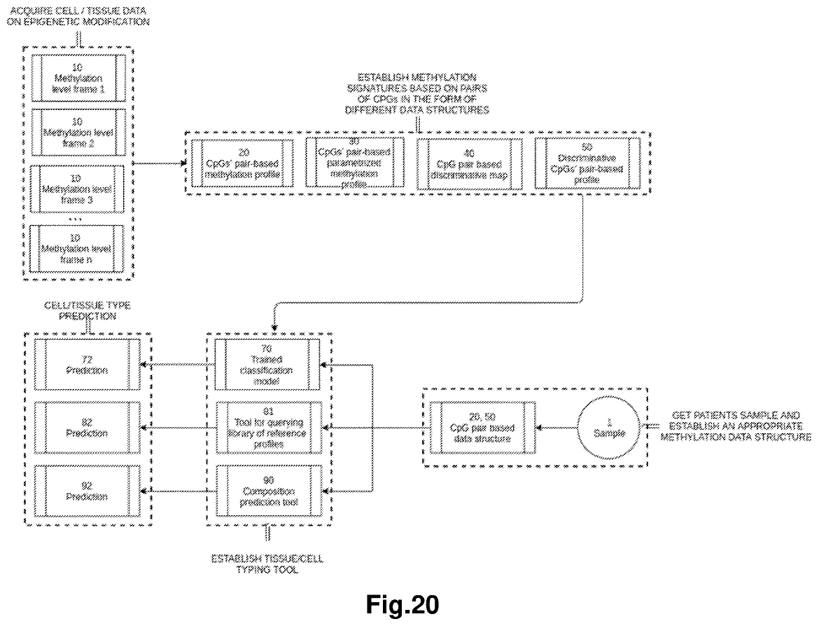

An exemplary overview of processing and analysis of methylation data according to the invention with a novel “CpG pair-based resolution” is illustrated in FIG. 20. It should be noted that the methods according to the invention are illustrated for methylation data, however it can be applied to any epigenetic modification data.

There are three components of the present invention that allows the invention to be industrially applicable and which are further claimed separately:
(i) preparing data objects necessary for creating cell/tissue type discrimination tools, namely, based on available methylation data, establishing specific data objects that at least contain methylation signatures based on adjacent CpG sites linked in pairs;
(ii) creating tools for cell/tissue type discrimination, namely creating a trained cell or tissue type classification model with the use of CpG pair-based discriminative profiles and/or creating a library of reference CpG pair-based discriminative profiles;
(iii) assessing/characterizing, namely typing or classifying a cell or tissue contained in a test sample or inferring DNAs composition in a test sample with the use of generated cell/tissue type discrimination tools.

In the first component (see FIG. 20), as many methylation (or other epigenetic modification) data as possible are collected from public data, such as the Genomic Data Commons (GDC), Gene Expression Omnibus (GEO) or ENCODE repository Also, data from private studies can be taken into account.

All data objects according to the invention are established based on specifically preprocessed existing methylation data. Some of them contain only genomic coordinates of all or only selected pairs of adjacent CpG sites (different types of maps). Other data objects according to the invention contain both genomic coordinates of all or only selected pairs of adjacent CpG sites as well as one or more numerical values related with each said pair, wherein one numerical value is always indicative of the co-methylation status or non co-methylation status in its related pair (different types of profiles, i.e., for one sample or for a group of samples).

In the second component (see FIG. 20), a classification model is trained with CpG pair-based discriminative profiles wherein each such profiles are inputted as a set of profiles of the same type as a training dataset along with appropriate labels that corresponds to a cancer or tissue or cell type. Also, a library of reference CpG pair-based discriminative profiles is built with data objects called reference CpG pair-based discriminative profiles wherein each such profile corresponds to a different cancer type or tissue type or cell type. Finally, a deconvolution tool is provided also with the same reference CpG pair-based discriminative profiles.

As it can be seen in FIG. 20, in the third component, digital data on epigenetic modification level are provided for test sample, namely a patient's DNA is converted and his/her methylation data is obtained, from a test sample for example, using EPIC microarrays, the Whole Genome Bisulfite Sequencing (WGBS) method from Illumina or the Reduced Representation Bisulfite Sequencing (RRBS) method or any other suitable method. Then, a processing of said methylation data according to the invention is applied to obtain a CpG pair-based discriminative profile of said test sample. Then the CpG pair-based discriminative profile of the test sample is inputted to the trained classification model or is used for quering a library in order to get the information about the type of the cell or tissue contained in the test sample and/or is inputted to the deconvolution tool to infer the sample compositions.

Processing of Raw Methylation Data into Data Objects Based on Pairs of Adjacent CpGs

It can be assumed that a methylation profile in the state of the art is a pure methylation data read from sequencing process, while a methylation signature is a specific information derived from typical methylation profile and is specific for tissue or cell origin. In some approaches, a methylation signature can be an information derived only for a part of the nucleic acid sequence, e.g, for a certain gene. A known methylation profile, as a data object, typically consists of genomic coordinates and associated methylation level value for each CpG site that has been read during sequencing. There is also another know type of object data called a methylation map which contains only genomic coordinates of CpG sites that have been read during measurement process.

The methods according to the invention provide new and inventive type of methylation signature (or signatures based on other types of epigenetic modification) derived from the nucleic acid sequence that has been read at once. It is a set of discriminative CpG pairs for which the methylation status between CpG sites that constitute such discriminative pair is differential for at least one type across different types of cells or tissues. It means that only one signature (one set of adjacent epigenetically modified bases) is derived for one cell or tissue type. Said methylation signature is specific for a tissue or a cell origin and is derivable from a basic data object, namely a “methylation profile 20 based on pairs of adjacent CpG sites”. It consists of genomic coordinates of selected adjacent CpG sites establishing each consecutive pair in said profile 20 as well as associated value representative for relation between methylation levels read for adjacent CpG sites in each pair. Advantageously, the relation between methylation levels read for adjacent CpG sites in each pair is represented by a numerical value which is a difference between methylation levels read for adjacent CpG sites in each pair. As a consequence, a CpG pairs-based methylation profile 20 is to some extent a set of compressed methylation information.

CpG pairs-based methylation profiles can be represented in multiple ways. CpG pairs-based methylation profiles according to the invention can be established at both population and individual levels. For example, at the population level (a set of samples) specific mathematical model parameters representing methylation level relation in a CpG pair can be determined, among which one parameter is indicative of co-methylation status or non co-methylation status. As a consequence, also CpG pairs-based parametrized profile 30 is to some extent a set of compressed methylation information. At the individual level (one sample), methylation level relation indicative of co-methylation status or non co-methylation status in a CpG pair can be determined directly from any kind of methylation assays (both qualitative or quantitative).

Data Objects with Derivable Methylation Signatures

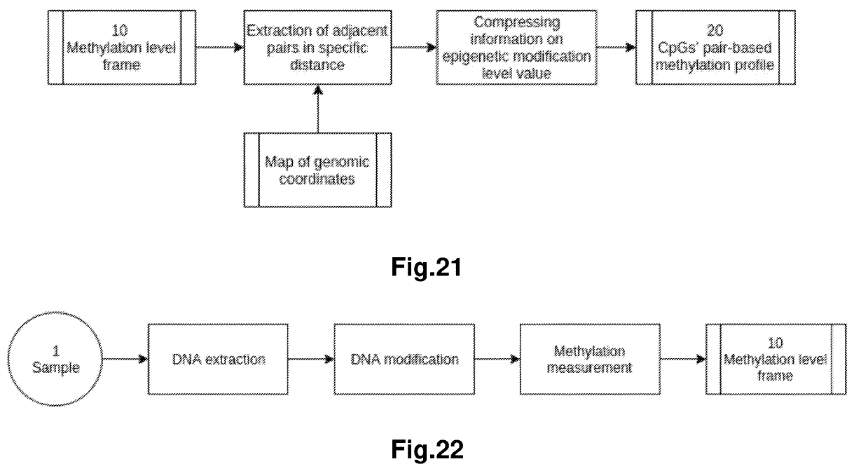

Now in reference to FIG. 21-26 all methods relating to the first mentioned component of the invention will be described. The first component relates to methods aiming at preparing data objects from which methylation signatures according to the invention are derivable or which finally contains only such methylation signatures according to the invention. All these methods are computer implemented methods.

The person skilled in the art will appreciate that such signature according to the invention can be received in general for any epigenetic modification, wherein epigenetic modification comprises one among methylation, hydroxymethylation, formylation or carboxylation of said base, and wherein the epigenetically modified base of the nucleic acid sequence is a methylated base, a hydroxymethylated base, a formylated base, or such base is substituted with carboxylic acid or a derivative thereof.

The first method, namely a method for constructing “CpG pair-based profile 20” or “profile based on pairs of adjacent epigenetically modified bases” of a tissue or a cell relates to generation of an object data which is a methylation profile at individual level, namely for one sample.

The second method, namely a method for constructing “parametrized profile 30 based on pairs of adjacent epigenetically modified bases” of a known type of tissue or cell relates to generation of an object data which is a methylation profile at human population level, namely for a set of samples.

Then, the third method, namely a method for providing a “discriminative map of cells or tissues 40 based on pairs of adjacent epigenetically modified bases” of a known cell type or tissue type within a specific application context relates to generation of an object data which is a discriminative methylation map 40 and comprises only information about final localization of appropriate parts of signature (markers), namely genomic coordinates of discriminative CpG pairs.

Then according to another method, namely a method for providing “cell or tissue type discriminative profile 50 based on pairs of adjacent epigenetically modified bases” of nucleic acid contained in a sample relating to a specific type of tissue or cell within a specific application context relates to generation of an object data which is a discriminative CpG pair-based profile 50 and comprises both information about final localization of biomarkers, namely genomic coordinates of discriminative CpG pairs as well their associated methylation level difference.

It should be emphasized that the invention is based on the usage of modified DNA material.

As shown in FIG. 21 the computer implemented method for constructing CpG pair-based profile 20 of a tissue or a cell according to the invention comprises a step of providing data on methylation level (in general epigenetic modification level) at specific CpG sites, wherein said epigenetic modification information is derived from nucleic acid containing epigenetically modified bases contained in a sample relating to said tissue or a cell. Such inputted data object can be called a “methylation level frame” 10. It contains a set of numerical values which are the methylation levels of an individual CpG sites. As mentioned earlier, the methylation level is understood herein as a numerical value indicative of measured methylation level or measured methylation status, depending on the applied measurement method.

In one embodiment, the step mentioned above involves reading appropriate data on methylation levels at specific CpG sites relating to said nucleic acid containing epigenetically modified bases from a database (see ‘acquisition step of public data o epigenetic modification’ in FIG. 20). It means that in one embodiment, the measurements of methylation level at CpG sites in said nucleic acid have been done by a third entity and made publicly available as databases for computer analysis or in the form of methylation level frames 10 or in another form that can be converted to methylation level frames 10.

As shown in FIG. 22, in another embodiment said step of providing data on methylation level at specific CpG sites for at least a part of a genome (in general, nucleic acid containing epigenetically modified bases) involves a series of steps performed on at least one biological sample 1. Said step of providing data on methylation level is also used for any test sample (see FIG. 20). In particular, it starts by DNA extraction and chemical DNA modification, namely by pretreating the genomic DNA of a sample by contacting the sample, or isolated DNA from the sample, with an agent, or series of agents that modifies unmethylated cytosine but leaves methylated cytosine essentially unmodified. Next, after such conversion, optionally, depending on the applied measurement method, amplification (not shown) of segments of the pretreated DNA is performed, said amplified segments representing the entire genome, or a portion thereof. Said segments comprises at least one dinucleotide sequence position corresponding to a CpG dinucleotide position in the corresponding untreated genomic DNA. Then, measuring of the pretreated nucleic acids is performed. Depending on the measurement method said material is analyzed to quantify a level of methylation at CpG positions or to qualify a status of the methylation at CpG positions. The results of the assessment are saved in electronic form, preferably as methylation level frames 10.

In one practical embodiment, the step of providing data on methylation level at specific CpG sites in nucleic acid containing epigenetically modified bases starts by a step of converting of DNA derived from at least one biological sample from the subject, said sample being associated with a specific type of cell or tissue, for example, obtained from bone tissue. As an example, such pretreatment of DNA involves the use of bisulfite reaction on DNA extracted from biological material (cells/tissues). Then, such chemically modified DNA is a batch material for all methylation measurement methods. Next, a step of measuring methylation levels is performed. For this purpose, the Illumina Infinium MethylationEPIC BeadChip measurement technology can be used which enables the measurement of approximately 850,000 CpGs in the human genome. The person skilled in the art will appreciate that this is one of possible technologies. Alternatively, any of the sequencing-based methods can be used, in particular Whole Genome Bisulfite Sequencing. As a result, a set of methylation level is obtained and saved as electronic data in a file, preferably as methylation level frames 10. Saved data comprises methylation level information on each CpG site.

Studies typically do not look at all of the 2.8*10^{A} 7 CpG sites available in the genome, but only at an interesting subset defined by the researcher or the technology that was used (e.g., EPIC arrays measure around 8.5*10⁵ of the predefined CpGs). It is obvious then that depending on the source of methylation data, methylation level frames 10 can comprise different number of methylation level measurements and requires often a particular preprocessing in order to make them uniform with other data used for a specific purpose according to the invention.

Next, once the data file(s) containing information on methylation levels at measured CpGs are read by the computer system, the method pass to a step of determining adjacent CpG pairs in nucleic acid containing epigenetically modified bases. In said step of adjacent CpGs pairs determination, for the n-number of CpGs for which methylation level was determined, CpG pairs are identified that satisfy the following conditions: a) chromosome localization condition [CHR CpGn] and advantageously b) nucleotide localization condition [MAPINFO CpGn].

To be able to link adjacent CpG site into pairs, first their genomic coordinates should be determined. For that purpose, each methylation level frame 10 is aligned with a reference genome (see Definitions) and as a consequence, a map of genomic coordinates is obtained (see the ‘map’ in FIG. 20). In practice said map is useful for more than one sample to the extent the methylation levels were measured by the same method. In other words, a step of annotating said nucleic acid containing epigenetically modified base to a reference genome is performed thereby determining genomic coordinates of each epigenetically modified base so as to generate a map of all epigenetically modified bases (shown in FIG. 20).

In practice, the first step of determination of pairs of adjacent CpGs (see extraction step in FIG. 20) involves checking two conditions: finding CpG pairs only within one chromosome and further CpG pairs which are localized at a distance less that a pre-defined threshold distance in bp units. Advantageously, the threshold distance is less than 50 in base pairs. As discussed earlier, said arbitrary parameter is advantageously chosen based on known correlation of methylation levels in human genome. Schematic diagram of the extraction of adjacent CpG sites is shown in FIG. 4a.

As an example, two CpG sites have been identified on the same chromosome namely, CpG 1 on the position no 100 and CpG 2 on the position no 115, which means that the chromosome localization condition is met. Moreover, the bp distance between CpG1 and CpG 2 is equal to 15, which means that nucleotide localization condition is also met. However, advantageously the threshold distance can be also less than 45, 40, 30, 35, 30, 25 or 25. It should be noted that different bp threshold distance results in different number of detected CpG pairs. Both mentioned conditions can be mathematically expressed as:
a) CHR CpGi=CHR CpGi+1; which means condition “a” is met if both CpGs [i and +1] are localized on the same chromosome;
b) MAPINFO CpGi+1—MAPINFO CpGi<bp threshold distance; which means condition “b” is met if distance between CpG i+1 a CpG is less than a bp threshold distance, for example 50 bp.

The result of the step of determination of pairs of adjacent CpGs is a k-element set of CpG (k<n, wherein n is a number of all assessed CpGs) pairs whose elements are localized close to each other (hereinafter referred to as the set of pairs of adjacent CpGs). In other words, as shown in FIG. 4A, within all available CpGs in a data file relating to a nucleic acid sequence under studies (their number depends on the chosen measuring technology (e.g. EPIC, WGBS), adjacent CpGs pairs are selected as close as possible to each other, preferably at a distance of no more than about 50 nucleotides. In particular, ‘two adjacent CpG sites’ means that there are no other measured CpG sites between the two sites constituting specific pair of adjacent CpGs pair.

Then, the method or constructing CpG pair-based profile 20 of a tissue or a cell according to the invention pass to a step of compressing information on epigenetic modification level value, iteratively for each pair of adjacent epigenetically modified bases among the determined set of pairs of adjacent epigenetically modified bases. It is done by converting each two separate epigenetic modification level values (for example, beta-value methylation rates received from EPIC) representative for each two separate epigenetically modified bases constituting pair of adjacent epigenetically modified bases into one single value representing compressed epigenetic modification information for a pair of adjacent epigenetically modified bases. Said one single value is an information indicative of an epigenetic modification status in each pair of adjacent epigenetically modified bases.

In particular the epigenetic modification status is one among at least co-epigenetic modification status in the pair of adjacent bases and non co-epigenetic modification status in the pair of adjacent bases.

In practice different mathematical conversion can be used which guarantees appropriate interpretation of the co-methylation status or non co-methylation status in a pair. Advantageously such mathematical operation on two numerical values can be subtraction. In other words, this step involves calculating epigenetic modification value reflective of difference between two epigenetically modified bases in each pair of said adjacent epigenetically modified bases.

As a result of said conversion, a change in the measurement scale and the new way of interpreting the methylation pattern is received. In details, the received methylation rate difference value is comprised from −1 to 1. For example, it allows to assess the direction of methylation change in nucleic acid molecule.

Finally, both genomic coordinates of adjacent CpGs' pairs and their associated delta methylation levels is saved. In this way a data object called CpGs' pair-based methylation profile 20 is generated.

It will be appreciated by the person skilled in the art, that such derived data set representing methylation levels of CpGs within CpG pairs is smaller in volume while preserving valuable information. It makes further analysis of the methylation data less complex and less time-consuming.

This kind of data object is necessary in the process of building automated cell type discrimination/sample characterization tools. It is also a data object that is derived firstly from a methylation level frame 10 of an unknown test sample and is further modified to a form which is appropriate for typing said unknown sample with the use of generated automated characterization (assessment) tools.

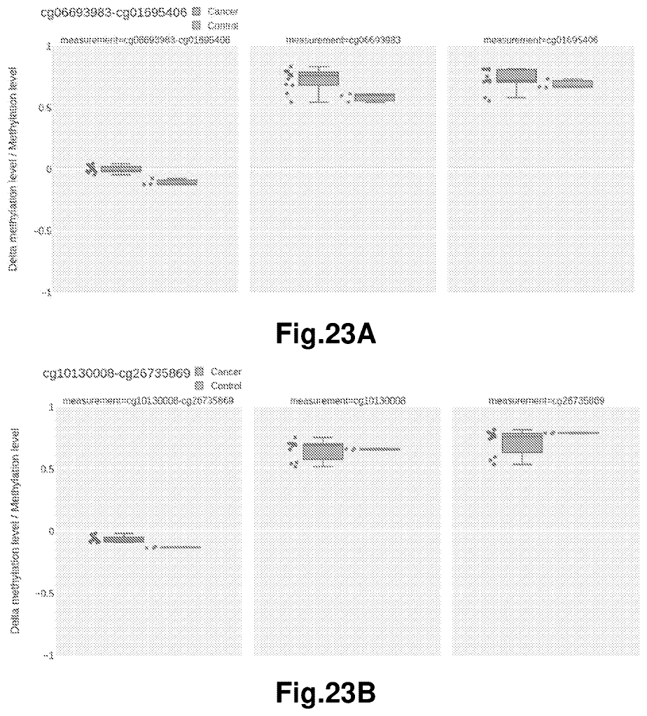

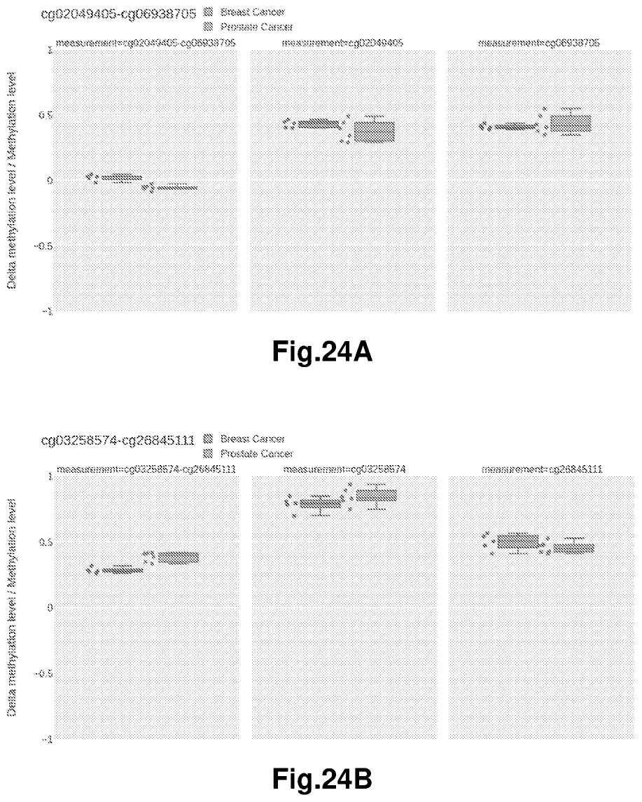

FIG. 23a-b and FIG. 24a-b show how crucial is the conversion of methylation levels for single CpG sites into one methylation difference value, namely why the CpG pair-based methylation profile 20 according to the invention containing such set of such methylation difference value is new and inventive. Delta beta value (difference of methylation levels in a pair of CpGs) even for one single CpG pairs may be a marker in specific context. To illustrate this phenomena methylation data from cfDNA isolated from the plasma of 3 healthy and 11 oncological patients (6 prostate cancer and 5 breast cancer) was used. For said samples methylation levels differences in a pair of CpGs (left graph) vs methylation values for said single CpG sites (middle graph and right graph) has been shown. It is clear that said exemplary CpG pairs shown in FIG. 23A and FIG. 23B are precise markers that generally distinguish healthy samples from cancer patients. One cannot draw such conclusions for methylation level information at a single CpG site. The same relates to exemplary CpG pairs shown in FIG. 24A and FIG. 24B which are precise markers that distinguish breast cancer from prostate cancer.

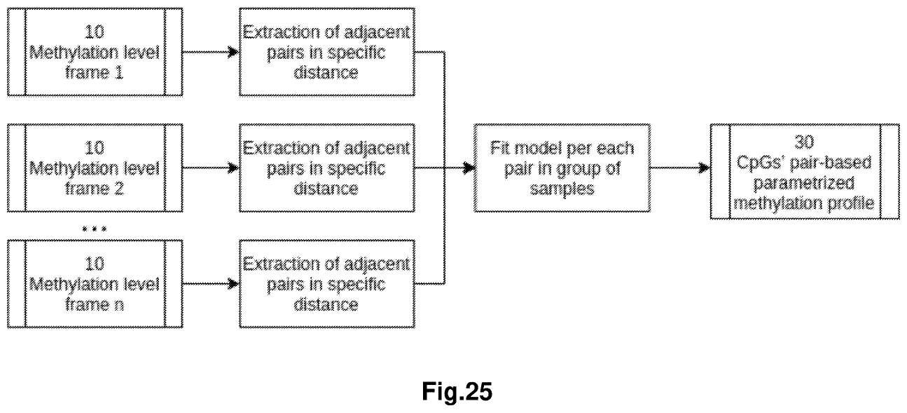

Now in reference to FIG. 25, a method for constructing parametrized profile 30 based on pairs of adjacent epigenetically modified bases of a known type of tissue or cell will be described.

Said method leads to another type of data object that has the same role for a group of samples as the previous object has for one sample, namely it contains for each pair of adjacent CpG sites only one numerical value that is indicative of the status in said pair, i.e co-methylation or non co-methylation. In other words, the parametrized profile 30 is a profile generated for a cell or tissue type at population level.

As it can be seen, the first two steps are almost similar with the previous method, except that here not one but several methylation level frames are required to be processed for a group of samples. The CpGs' pair-based profile is now a parametrized one, and is generated based on epigenetic modification information derived from epigenetically modified base-containing nucleic acid contained in a group of at least one sample, wherein each sample relates to the same known type of said tissue or said cell.

The first step of the method is: for each sample in said group of at least one sample digital data on epigenetic modification level measurements for epigenetically modified bases contained in said nucleic acid in said sample relating to said tissue or said cell is provided. In practice it means that several data objects with methylation measurements information for a certain type of tissue or cell are downloaded again from databases or generated in laboratory for further processing, and if required, converted to methylation level frames (with numerical values for each epigenetically modified base).

Then for each sample in said group of at least one sample a set of pairs of adjacent epigenetically modified bases is determined, wherein each pair of adjacent epigenetically modified bases consists of two adjacent epigenetically modified bases localized within one nucleic acid molecule so as to generate a map of adjacent epigenetically modified bases. Again, pairs of CpG are selected based on two conditions which can be mathematically expressed as:
a) CHR CpGi=CHR CpGi+1; which means condition “a” is met if both CpGs [i and i+1] are localized on the same chromosome;
b) MAPINFO CpGi+1—MAPINFO CpGi<bp threshold distance; which means condition “b” is met if distance between CpG i+1 a CpG is less than a bp threshold distance, for example 50 bp.

The result of the step of determination of pairs of adjacent CpGs for each sample in said group of at least one sample is a k-element set of CpG (k<n, wherein n is a number of all assessed CpGs) pairs whose elements are localized close to each other (hereinafter referred to as the set of pairs of adjacent CpGs). In other words, as shown in FIG. 4A, within all available CpGs in a data file relating to a genome under studies (their number depends on the chosen measuring technology (e.g. EPIC, WGBS), adjacent CpGs pairs are selected as close as possible to each other, preferably at a distance of no more than about 50 nucleotides. In particular, ‘two adjacent CpG sites’ means that there are no other measured CpG sites between the two sites constituting specific pair of adjacent CpGs pair.

Then the method passes to a step of fitting a parametrized mathematical model to said digital data on epigenetic modification level measurements associated with each pair of adjacent epigenetically modified bases so as to determine at least first single model parameter representing compressed epigenetic modification information for a pair of adjacent epigenetically modified bases for a group of at least one samples of said known type of tissue or cell, wherein said first single model parameter is indicative of an epigenetic modification status in each pair of adjacent epigenetically modified bases.

It should be noted, that depending on the applied mathematical model, there is one or more model parameters. However, there is always one parameter that is a single numerical value indicative of an epigenetic modification status in each pair. The other model parameters can relate for example (as described below) to statistical significance level.

According to the invention the epigenetic modification status is one among at least co-epigenetic modification status in the pair of adjacent bases and non co-epigenetic modification status in the pair of adjacent bases.

In one embodiment, the step of fitting a parametrized mathematical model comprises: for each pair of adjacent epigenetically modified bases separately, estimating a regression model such that:

$$MetLevel ∼ intercept + {β}_{i} * POS$$

$$POS = \{ \begin{matrix}0 & if CpG = first \\ 1 & if CpG = successive\end{matrix}$$

wherein intercept and βi are model parameters, the parameter POS being an exogenous variable taking values of 0 if the epigenetically modified base in a pair is a first one, or 1 if there is a successive one.

In one embodiment, the received model parameter can be interpreted as follows: if βi≠0 the non co-epigenetic modification status is further one among negative non co-epigenetic modification status for βi<0 and positive non co-epigenetic modification status for βi>0. However, in another more general embodiment, the received model parameter can be interpreted as follows: if βi≠0 then it is indicative of the non co-epigenetic modification status.

As mentioned earlier, another model parameter is received in said case, namely the statistical significance assessment of the value of the pi parameter is performed using Student's test to calculate and save the value of empirical probability (p-value). Said second model parameter can be used to set a threshold value for discriminating pi parameter between βi=0 and βi≠0, as to receive as an interpretation of the generated parametrized profile only a combination of co-methylation status and non co-methylation status in a pair of adjacent CpGs.

The regression model is any regression model that links the position converted to binary form (namely 0 for the first epigenetically modified base and 1 for successive one) of a CpG to its methylation level in a given pair (e.g., univariate, multivariate, mixed effect model, Huber regression, etc.).

In another embodiment the step of fitting a parametrized mathematical model comprises first determining, separately for each first and each successive epigenetically modified base in each same pair of adjacent epigenetically modified bases across said group of samples, a confidence interval for the mean epigenetic modification level such that:

$$P ( \overline{X} - {t}_{\text{?}} \frac{S}{\sqrt{n}} < μ < \overline{X} + {t}_{\text{?}} \frac{S}{\sqrt{n}} ) = 1 - \text{?} ,$$

$$\text{?} \text{indicates text missing or illegible when filed}$$

wherein alfa is a predefined parameter, advantageously equal to 0.05, n—the number of samples in the set of samples, S—standard deviation from the epigenetic modification level in said group of samples for each epigenetically modified base in the pair, t1-alfa/2 the quantile of order 1-alfa/2 of student's t-distribution with n−1 degrees of freedom.
And secondly determining a model parameter being the distance βi between the confidence interval calculated for the first epigenetically modified base and the confidence interval calculated for the successive epigenetically modified base in the pair using a selected measure of distance.

In one embodiment, said measure of distance can be the Chebyshev measure:

$${β}_{i} = {D}_{Chebyshev} ( CI ( {CpG}_{first} ) , CI ( {CpG}_{successive} ) ) = max ( \vert CI ( {CpG}_{first} ) , CI ( {CpG}_{successive} ) \vert )$$

CI—confidence interval, CI(CpG first) confidence interval for mean methylation level of first CpG site, CI(CpG successive)—confidence interval for successive CpG site mean methylation level.

This approach with the Chebyshev measure has been used also in background studies (see section KEY FINDINGS RELATING TO CO-METHYLATION AND NON CO-METHYLATION STATUS IN PAIRS OF ADJACENT CpGs). In particular, in order to identify non co-methylated and co-methylated pairs of CpG sites a Python-based script was developed that, firstly mapped all CpG spaced less than 50 bp starting from the first base of each chromosome (as mentioned earlier the graphical illustration of this selection is described in FIG. 4A). Secondly, said Python-based script filtered for the CpG pairs with more than about 0.3 beta-value difference between CpGs (calculated as Chebyshev distance between two points defined as 95% confidence intervals), measured with standard deviation<0.1 across all EPIC arrays in the entire data set.

In another embodiment, the step of fitting a parametrized mathematical model comprises first determining, separately for each first and each successive epigenetically modified base in each same pair of adjacent epigenetically modified bases across said group of samples, mean level of epigenetic modification, secondly determining a model parameter βi being the absolute value of difference between mean levels of epigenetic modification of the epigenetically modified bases for the first epigenetically modified base based and the successive epigenetically modified base.

In the above mentioned embodiment, another model parameter is calculated, namely the method further comprises testing significance of said calculated difference between mean levels of epigenetic modification of the epigenetically modified bases using statistical tests such as for example t-test, ANOVA, Mann-Whitney U or Kruskal-Wallis to calculate and save the value of empirical probability (p-value) for each pair of adjacent epigenetically modified bases. Again, said second model parameter can be used to set a threshold value for discriminating pi parameter between βi=0 and βi≠0, so as to receive as an interpretation of the generated parametrized profile only a combination of co-methylation status and non co-methylation status in a pair of adjacent CpGs.

It should be noted that for both alternative mathematical models data conversion allows to receive a model parameter being an absolute value. As a consequence, only two types of status in pairs of adjacent CpG sites can be determined, namely co-methylated and non co-methylated (also called discordantly methylated) and the information about the order of the change of the methylation level between two CpG sites, namely the sign of the assessed change is lost.

Finally, the method ends by saving data, namely by saving both genomic coordinates of adjacent CpGs' pairs and their associated at least first single model parameter (namely βi or βi and p-value). In this way a data object called CpGs' pair-based parametrized methylation profile 30 is generated.

It will be appreciated by the person skilled in the art, that such derived data set on methylation level is smaller in volume while preserving valuable information. As for the previous data object (namely the methylation profile for one sample) it makes further analysis of the methylation data less complex and less time-consuming.

This kind of data object is also necessary in the process of building automated cell type discrimination tools.

It is also clear that such new derived form of methylation data provides information about at least two different methylation statuses in pairs of adjacent CpGs. It means that in practice methylation data derived by the method according to the invention can be also converted into binarized data or three states data. As a consequence, at least two different types of pairs of adjacent CpG based on methylation value difference can be discriminated, namely those pairs within which there is no methylation level value change between sites, as well those pairs within which there is methylation level value change between sites, in particular two further types of pairs with the methylation value change can be determined by checking the sign of the calculated difference value.

The above discussed embodiment presents the most popular mathematical implementation of the methylation data processing according to the invention, however person skilled in the art will appreciate that other known mathematical models can be implemented in the computer system in order to convert, for a group of samples, the information on methylation level from two numerical values representatives for two sites into a single parameter (single numerical value) indicative of co-methylation status or non co-methylation status for a pair of adjacent CpGs.

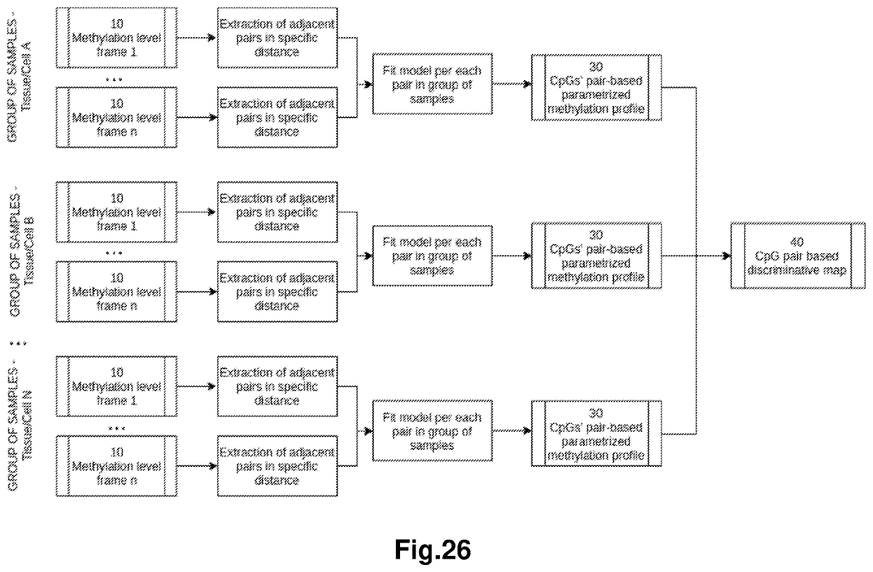

Now in reference to FIG. 26, a method for providing a discriminative map 40 of cells or tissues based on pairs of adjacent epigenetically modified bases of a known cell type or tissue type within a specific application context will be described.

The specific application context is given by the type and number of parametrized profiles being compared and processed. At the beginning at least a first parametrized profile 30 based on pairs of adjacent epigenetically modified bases for the first known type of cell or tissue is provided.

Then at least a second parametrized profile 30 based on pairs of adjacent epigenetically modified bases for at least a second known type of cell or tissue is provided. Advantageously, only one first parametrized profile is enough to generate a discriminative map 40 of cells or tissues based on pairs of adjacent epigenetically modified bases of a known cell type or tissue if compared with one second parametrized profile 30 relating to another known cell type or tissue. For example, the discriminative map 40 can be generated for a certain pathological type of the lung tissue in the context of the healthy lung tissue (the specific application context is given by said two parametrized profiles of healthy and pathological lung tissue).

In another embodiment, if there are multiple different pathologies for one tissue type, then the method according to the invention allows to generate a discriminative map in the context of possible types of diseases of said tissue. Such a case is shown in FIG. 13.

In another embodiment the specific context can be given by only healthy tissues of different origin. Such a case is shown in FIG. 18. In yet another embodiment the specific context can be given by grouping different types of tissues (healthy and pathological) of the same origin with different types of tissues (healthy and pathological) of another origin.

The method further comprises determining and comparing the epigenetic modification status contained in the model parameters for each pair of adjacent epigenetically modified bases present in at least both parametrized profiles 30.

Next, if in at least one parametrized profile there is a pair for which the status is not repeatable for at least one other parametrized profile among all other parametrized profiles 30 (namely, the status is unique for said pair in at least one parametrized profile) then such a pair is extracted to be a part of said discriminative map of cells or tissues under generation.

If the specific application context is given by grouping different types of tissues (healthy and pathological) of the same origin with different types of tissues (healthy and pathological) of another origin, the discriminative map 40 can comprise (among others) CpG pairs which are stable (having repeatable first type of the methylation status in a pair) for the tissues of the same origin (within one group) but discriminative in the context of the second group of tissues having another common origin (having repeatable, stable second type of the methylation status in said pairs).

In general, the generated discriminative map 40 can be analyzed for different types of stability of methylation statuses in pairs of adjacent CpG sites contained in if necessary.

Finally, the method comprises a step of saving genomic coordinates for the extracted pairs of adjacent epigenetically modified bases in the discriminative map 40 of cells or tissues within said specific application context.

The construction of a CpG pair-based discriminative map 40 is generally based on iterative comparison of consecutive statuses for consecutive CpG pairs stored in at least two data files being parametrized profiles 30 according to the invention. In another embodiment the construction of a CpG pair-based discriminative map 40 can be based on usage of conventional statistical methods. In such a case the input of said statistical methods are data objects which are CpG pairs-based profiles 20 which are selected so as to constitute specific application context. For example, the extraction of discriminative pairs of CpG sites (also called differential methylation pairs) can be performed by comparing delta methylation levels between groups of samples using statistical test, such as: ANOVA, Kruskal-Wallis test, Man-Whitney U test or t-test. Next genomic coordinates for the extracted pairs of adjacent epigenetically modified bases are saved in the discriminative map 40 of cells or tissues within said specific application context

Herein, the term “discriminative map” 40 should be understood as a set of genomic coordinates for at least one pair of adjacent CpG sites that are assumed to be biological markers within said specific application context.

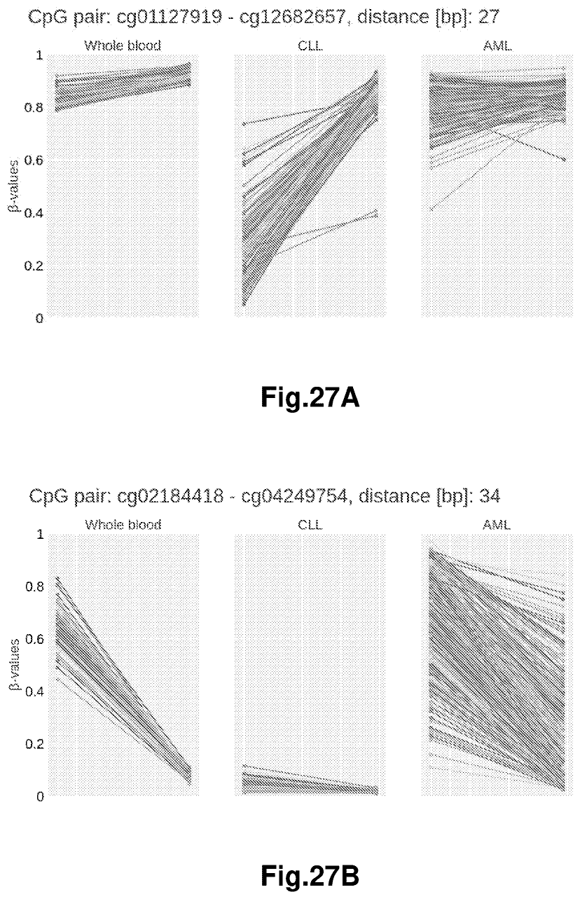

A visual example of discriminative “non co-metylated” status and discriminative co-methylated status for chosen CpG pairs across two different types of blood cancer acute myeloid leukemia (AML) and chronic lymphocytic leukemia (CLL) can be seen in the FIG. 27A-27B.

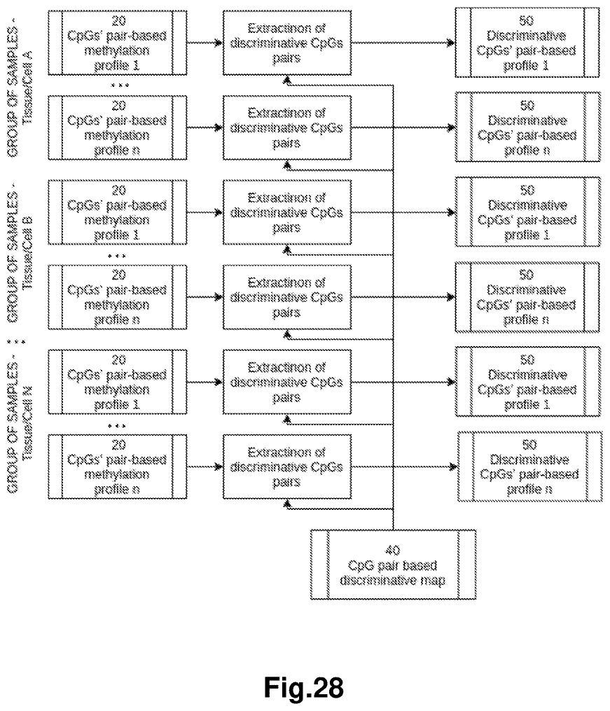

Now in reference to FIG. 28 a computer implemented method for providing cell or tissue type discriminative profile 50 based on pairs of adjacent epigenetically modified bases of nucleic acid contained in a sample within a specific application context.

This method leads to generation of a data object that is required for training an automated classification tool 70, i.e. a neural network. Said discriminative CpG pairs-based profile 50 is associated with one sample relating to a known type of tissue or cell. In practice discriminative CpG pairs-based profiles 50 are generated for all available samples relating to a known type of tissue or cell. Such data, along with their known labels (known information about the origin/type of sample) constitutes a training data set for said automated classifier 70 (automated classification tool 70).

In another embodiment this method leads to generation of a data object that is required for a test sample 1 before digital data relating to it is inputted to a characterizing/assessing tool (70,70′,80,90) according to the invention.

The method for providing cell or tissue type discriminative profile 50 based on pairs of adjacent epigenetically modified bases of nucleic acid comprises the following steps:

Firstly, for a sample, its profile 20 based on pairs of adjacent epigenetically modified bases is provided, said profile being generated with the use of the method as described earlier in reference to FIG. 21, Secondly, then a discriminative map of cells or tissues 40 based on pairs of adjacent epigenetically modified bases within a specific application context is provided, said discriminative map 40 is generated with the use of the method described in reference to FIG. 26. In one embodiment said discriminative map 40 can be obtained using data objects being CpG methylation profiles 20 and statistical methods as described above.
Finally, CpG pairs in the profile 20 and in the discriminative map 40 are compared and only for each pair contained in said discriminative map of cells or tissues 40, genomic coordinates of adjacent epigenetically modified bases constituting said pairs as well as compressed epigenetic modification information relating to said adjacent epigenetically modified bases in the form of one single numerical value are saved into the discriminative CpG pairs-based profile 50.

Herein, the term “discriminative profile” 50 should be understood as a set of at least one pair of adjacent CpG sites for which the compressed epigenetic modification information has been calculated and saved, that are assumed to be biological markers within said specific application context.

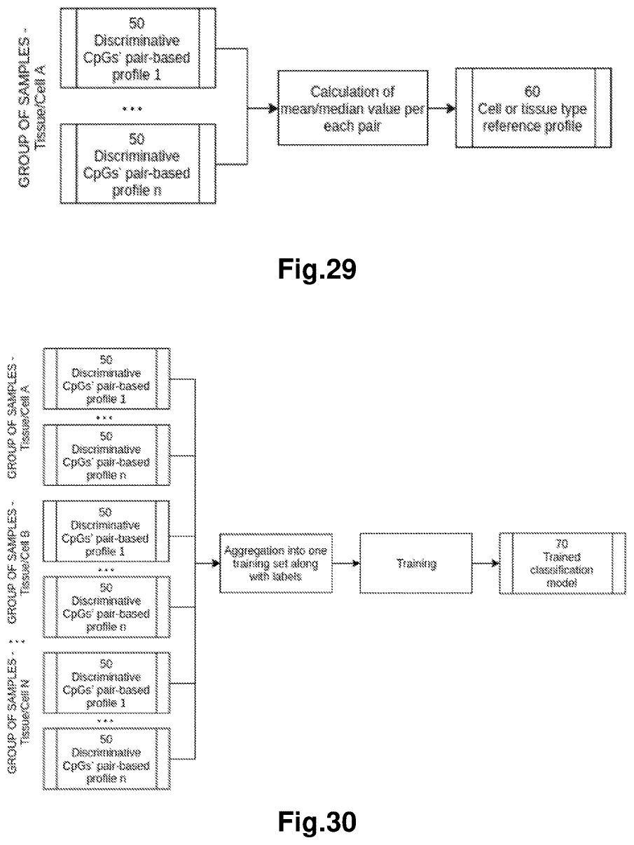

Finally, a method for providing cell or tissue type reference profile 60 based on pairs of adjacent epigenetically modified bases of nucleic acid will be described in reference to FIG. 29.

This method leads to generation of a data object that is required for creating another automated typing tool 80, namely a searchable database (a library) of reference profiles 60 coupled with a typing routine. Said reference profile 60 is generated within a specific application context for a population of samples containing the same type of tissue or cell and contains genomic coordinates of adjacent epigenetically modified bases and a mean value of compressed epigenetic modification information across said population of samples.

The method for constructing reference discriminative profile 60 of a known type of cell or tissue type based on pairs of adjacent epigenetically modified bases comprises the step of providing a set of discriminative profiles 50 within a specific application context obtained by the method describe d in reference to FIG. 28 for at least a group of two samples of the same known type of the tissue or cell. Next, for each pair of adjacent epigenetically modified bases in said discriminative profiles 50, one among a mean value and median resulting from compressed epigenetic modification information in all said samples in said group of at least two samples is calculated. Namely delta beta values resulting from all profiles are taken into account when calculating one among a mean value and median for each pair of adjacent CpG pairs. Finally, genomic coordinates of each pair of adjacent epigenetically modified bases and its respective mean or median value of the compressed epigenetic modification information is saved into an object called a reference discriminative profile 60 of a known cell or tissue type based on pairs of adjacent epigenetically modified bases within a specific application context.

### Automated Tools for Cell or Tissue Typing

Now the second component of the industrial application of the invention will be described, namely computer implemented tools appropriate to asses or characterize, e.g to classify and type unknown cell or tissue type or to type a composition of cells or tissues.

### Cell or Tissue Type Prediction Model

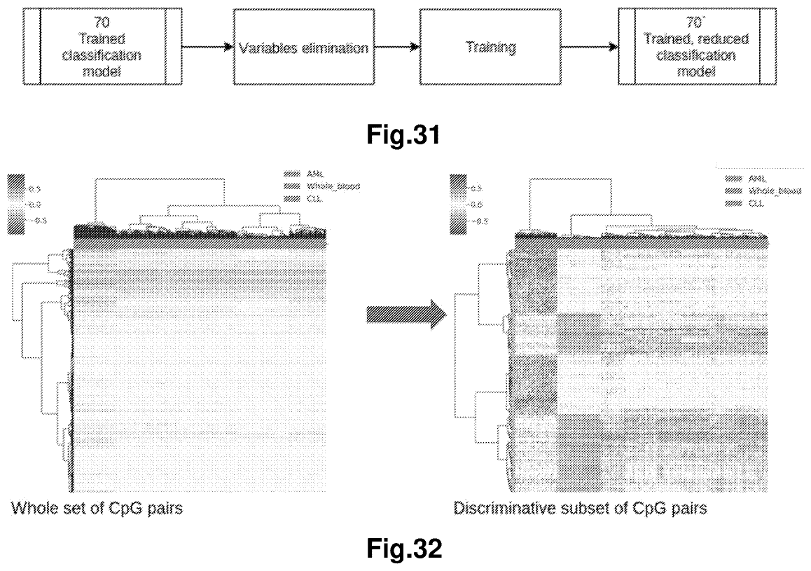

In reference to FIG. 30-31 a computer implemented method for providing a trained model 70 for cell or tissue type classifying will be described. The method comprises: providing a training data set by providing a first set of cell or tissue type discriminative profiles 50 based on pairs of adjacent epigenetically modified bases and providing at least a second set of cell or tissue type discriminative profiles 50 based on pairs of adjacent epigenetically modified bases, said discriminative profiles 50 being obtained by the method described in reference to FIG. 28. Moreover, labels associated with said discriminative profiles 50 are provided.

Then the classification model 70 is trained with the above mentioned data training set so as it is configured to classify an input cell or tissue type discriminative profile based on pairs of adjacent epigenetically modified bases into at least two classes.

If the number of available samples is large enough (in practice >50-100 for each analyzed tissue/cell/tumor type) their methylation signatures according to the invention can be extracted, namely discriminative profiles 50 of said samples with known type of tissue or cell, can be entered into supervised machine learning models (classifiers) to automate the tissue/cell/disease type prediction process.

In the present method of discriminating/predicting a type of cell using created discriminative profile based on CpG pairs-based methylation information, any classifier can be used, but most often models indicated below are used to assess the significance of explanatory variables, e.g.
a) decision tree (https://scikit-learn.org/stable/modules/tree.html);
b) ensemble models, using various methods of building a decision rule
(https://scikit-learn.org/stable/modules/ensemble.html#forest,
https://scikit-learn.org/stable/modules/ensemble.html#gradient-boosting,
https://xgboost.readthedocs.io/en/stable/tutorials/model.html);
c) parametric models, e.g. generalized linear models (https://www.statsmodels.org/stable/glm.html);

The built prediction model 70 can be used to eliminate unnecessary variables (reduce the number of CpG pairs in the training data sets used for prediction, i.e. the number of necessary measurements), e.g. using recursive feature selection (https://scikit-learn.org/stable/modules/feature_selection.html#ref) or information criteria, e.g. BIC (Bayesian information criterion) or AIC (Akaike information criterion) for parametric models. As a result, a reduced prediction model 70 is obtained that uses the least possible information (variables) while maintaining maximum accuracy (it is a form of compromise between the complexity and the accuracy of the trained prediction model).

As it can be seen in FIG. 31, said computer implemented method for constructing reduced discriminative map of cells or tissues 40′ based on pairs of adjacent epigenetically modified bases of known types of tissue or cell for specific application comprises: providing the classification model 70 received by the method described in reference to FIG. 30, providing a training data set being a first and at least a second set of cell or tissue type discriminative profiles 50 based on pairs of adjacent epigenetically modified bases for at least two different types of cell or tissue obtained by the method described in reference to FIG. 28 along with associated labels, eliminating unnecessary pairs of adjacent epigenetically modified bases in the training data set by further training said classification model 70 with the use of a feature selection method, saving reduced discriminative map 40′ and saving said further trained reduced model 70′. Said reduced discriminative map 40′ (always within a specific application context) can be used to build other automated tools for sample characterizing according to the invention, in particular to build a library 80′ of reduced reference discriminative profiles 60′, which can be used with the querying tool 81 and the deconvolution tool 91.

The predictive power of classification model may be described as classification performance metric e.g.: accuracy, precision, recall or f-beta scores using preprepared tests set.

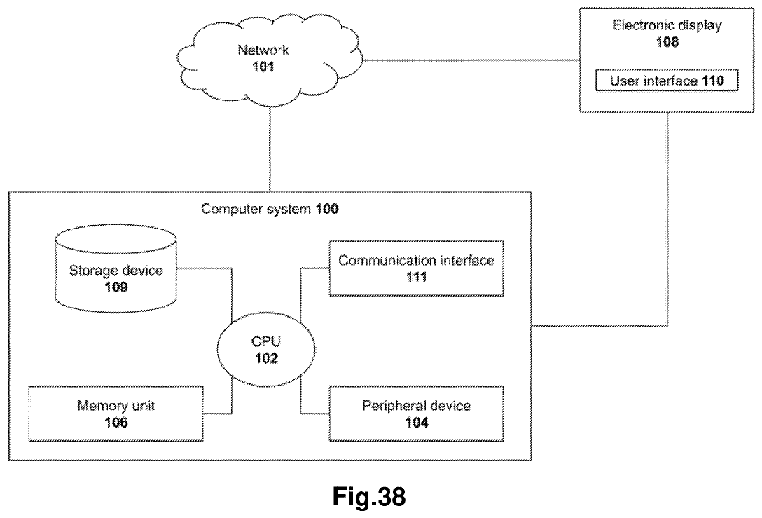

The result of said sample characterizing tool 70, 70′ can be called in general a prediction score X. The automated tool for cell or tissue classifying (70,70′) according to the invention is implemented by the computer system as described later in reference to FIG. 38. In said case said automated tool for cell or tissue classifying is saved in appropriate memory of said system.

During studies it was tested whether machine learning based model can be developed for precise typing of malignant tissues. Exemplary tests were performed first with signatures based on non co-methylation patterns (i.e., using methylation signatures which contained only discriminative pairs with non-co epigenetic modification status). The person skilled in the art will appreciate that even using limited EPIC microarray data, thousands of non co-methylated loci for specific cell/tissue type which then changed in cancer tissue are identified with the method according to the invention. Relatively large number of features always increase model complexity and reduces potential model utility. Thus, to reduce the number of the features two-steps approach to select the non co-methylated CpG pairs that maximize effectiveness of prediction can be developed. During studies, in the first step of the analysis, all EPIC microarrays methylation profiles that include: acute myeloid leukemia (AML) n=458, chronic lymphocytic leukemia (CLL) n=114 and healthy blood n=112 were divided into training and tests sets using 70:30 stratified sampling. Using training set it was identified 156 CpG pairs (beta>0.3, p-value<=0.05), referred to as discriminative subset of CpG pairs, namely discriminative profiles 50 based on CpG pairs (FIG. 32).

Then recursive features elimination with cross-validation (RFECV) from scikit-learn library using XGBClassifier (42) from XGBoost library (43) was applied to select the minimal number of features providing highest model classification efficiency. The number of variables required to prediction was reduced from 156 to 17.

The accuracy of this model in testing set (206 samples: 138 AML 34 CLL, 34 whole blood) was close to 99% and f1-score equal to 0.99, 1.0 and 0.97 respectively for AML, CLL and healthy blood samples groups. Using the same machine learning pipeline) all six main analyzed WBC types were distinguished with 100% accuracy.

### Reference Library and Searchable Reference Library

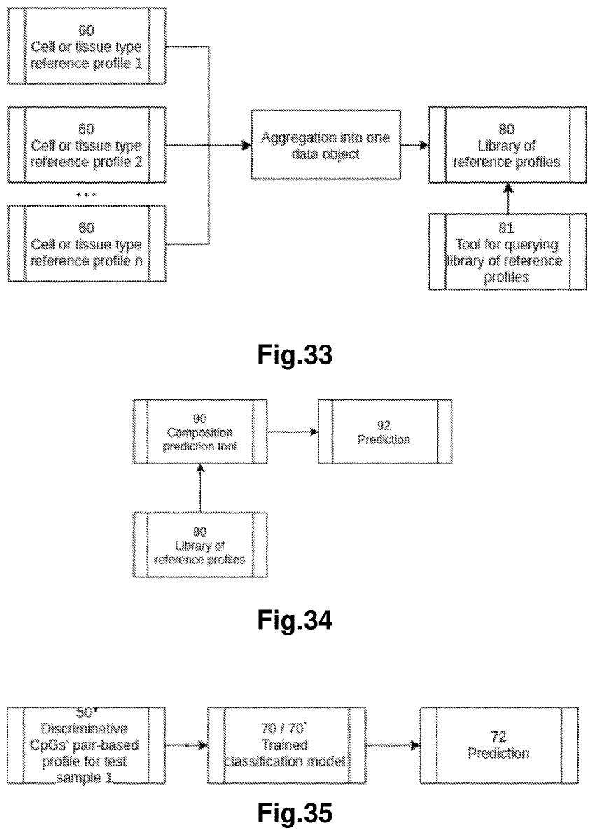

Now another automated cell or tissue typing tool will be described in reference to FIG. 33, namely a library 80 of reference discriminative profiles 60 for given cell or tissue types.

The method for generating a library 80 of reference discriminative profiles 60 based on pairs of adjacent epigenetically modified bases for known types of tissue or cell for specific application comprises:
a) providing a first reference cell or tissue type discriminative profile 60 within a specific application context based on pairs of adjacent epigenetically modified bases for a first known type of cell or tissue, said reference discriminative profile 60 being generated with the method described in reference to FIG. 29
b) providing at least a second reference cell or tissue type discriminative profile 60 within said specific application context based on pairs of adjacent epigenetically modified bases for at least a second known type of cell or tissue, said at least second reference discriminative profile 60 being generated with the method described in reference to FIG. 29
c) storing said at least two reference discriminative profiles 60 based on pairs of adjacent epigenetically modified bases as a library 80 within a specific application context.

In practice, such a tool can be built and used instead machine learning based model, in particular when interpretability is a desired classifier feature. Thus, it is possible to use other simple computer implemented algorithm for comparison of created and stored reference CpG pairs-based profiles 60 for known cell and tissue types with a CpG pairs-based discriminative profile 50 of an unknown cell or tissue. For applicability of such automated tool also a searching and comparing algorithm coupled with said library is required (as shown in FIG. 33). Such algorithms are known in priori art as distance metrics and can be for example: geometric distance, Chebyshev distance, cityblock distance or Minkowski distance. The prediction result of such a typing tool has been schematically shown as a block 81 with typed the most similar reference profile found in the library. The result of said sample characterizing tool can be called a prediction score λ.

Such searchable digital reference library 80 of reference discriminative profiles 60 based on adjacent epigenetically modified bases can be a tool utilizing distance metrics to identify the most similar reference profile 60 in library 80 to inputted sample. The automated tool for cell or tissue typing (80,81) according to the invention is implemented by the computer system as described later in reference to FIG. 38. In said case in appropriate memory of said system a reference library 80 of reference discriminative profiles 60 is saved to allow extraction of compressed methylation levels required for distance metrics calculation.

### Reference Deconvolution Model

Now another type of automated tool for sample characterizing(assessing), i.e. for composition typing will be described in reference to FIG. 34.

It is known in the art the use of deconvolution algorithm 90 (also called composition prediction tool 90) to type the sample's cell or tissue composition. Such composition typing tool based on deconvolution algorithm can be built also with the use of the library 80 of the reference discriminative profiles 60 according to the invention.

A deconvolution process can be used to determine fractional contributions (e.g., percentage) for each of the cell or tissue types for which cell or tissue specific methylation levels (i.e unique methylation signatures) are known.

The principle of methylation deconvolution can be illustrated using a single CpG pair to determine a composition of a DNA mixture from an organism. Assume that in tissue A, delta methylation level for single CpG pair namely methylation level difference (MLD), is 1 and in tissue B is −1. In this example, delta methylation level refers to size and direction of methylation change between CpGs in single CpG pair.

If the DNA mixture C is composed of tissue A and tissue B and the overall delta methylation level of the DNA mixture C is 0, we can deduce the proportional contribution of tissues A and B to the DNA mixture C according to the following formula:

$$MLDC = MLDA \cdot a + MLDB \cdot b$$

where MLDA, MLDB, MLDC represent the MLD of tissues A, tissue B and the DNA mixture C, respectively; and a and b are the proportional contributions of tissues A and B to the DNA mixture C. In this particular example, it is assumed that tissues A and B are the only two constituents of the DNA mixture. Therefore, a+b=100 percent. Thus, it is calculated that tissues A and B contribute 50 percent and 50 percent, respectively, to the DNA mixture.

The MLD in tissue A and tissue B can be obtained from samples of the organism or from samples from other organisms of the same type (e.g., other humans, potentially of a same subpopulation). If samples from other organisms are used, a statistical analysis (e.g., average, median, geometric mean) of the delta methylation level of the samples of tissue A can be used to obtain the delta methylation level MLDA, and similarly for MLDB.

CpG pair can be chosen to have minimal inter-individual variation, for example, less than a specific absolute amount of variation or being within a lowest portion of genomic sites tested. For instance, for the lowest portion, embodiments can select only genomic sites having the lowest 10 percent of variation among a group of genomic CpG pair tested. The other organisms can be taken from healthy persons, as well as those with particular physiologic (e.g. pregnant women, or people with different ages or people of a particular sex), which may correspond to a particular subpopulation that includes the current organism being tested.

The other organisms of a subpopulation may also have other pathologic conditions (e.g. patients with hepatitis or diabetes, etc.). Such a subpopulation may have altered tissue-specific CpG-pair based methylation patterns for various tissues. The CpG-pair based methylation pattern of the tissue under such disease condition can be used for the deconvolution analysis in addition to using the methylation pattern of the normal tissue. This deconvolution analysis may be more accurate (with reduced differences between estimated and real cell type proportions) when testing an organism from such a subpopulation with those conditions. For example, a cirrhotic liver or a fibrotic kidney may have a different CpGpair based methylation pattern compared with a normal liver and normal kidney, respectively. Thus, if a patient with liver cirrhosis was screened, a more accurate estimate of sample composition can be made by including cirrhotic liver tissue from a patient suffering from cirrhosis as one of the candidates contributing DNA to the DNA mixture, together with the healthy tissues of other tissue types.

It will be appreciated by the person skilled in the art that more genomic CpG pairs (e.g., 10 or more) may be used to determine the constitution of the DNA mixture when there are more potential candidate tissues. The accuracy of the estimation of the proportional composition of the DNA mixture is dependent on a number of factors including the number of genomic sites, the specificity of the methylation changes at those genomic CpG pairs (also called “CpG pairs”) to the specific tissues, and the variability of the sites across different candidate tissues and across different individuals used to determine the reference tissue-specific levels. The specificity of a site to a tissue refers to the difference in the delta methylation level of the CpG pairs between the particular cell or tissue type and other cell or tissue types.

The larger the difference between their delta methylation levels, the more specific the site to the particular tissue would be. For example, if a delta methylation level in CpG pair is close to 1 in the liver and is equal to 0 in all other tissues, this site would be highly specific for the liver. Whereas, the variability of a site across different tissues can be reflected by, for example, but not limited to, the range or standard deviation of methylation of the site in different types of tissue. A larger range or lower standard deviation would allow a more precise and accurate determination of the relative contributions of the different cell or tissues to the DNA mixture mathematically

Here, we use mathematical equations to illustrate the deduction of the proportional contribution of different cell or tissues to the DNA mixture. The mathematical association between the delta methylation levels of the different CpG pairs in the DNA mixture and the delta methylation levels of the corresponding CpG pairs in different tissues can be expressed as:

$$MLDi = \sum k ( pk \cdot MLDik )$$

where MLDi represents the delta methylation level of the i-th CpG pair in the DNA mixture; pk represents the proportional contribution of tissue k to the DNA mixture; MLDik represents the delta methylation level of the i-th CpG pair in the tissue k.

Additional criteria can be included in the algorithm to improve the accuracy. For example, the aggregated contribution of all tissues can be constrained to be 1.

Furthermore, all the tissues contributions can be required to be non-negative: pk greater than or equal to 0.

The automated tool for deconvolution 90 according to the invention is implemented by the computer system as described later in reference to FIG. 38. In said case in appropriate memory of said system a reference library 80 of reference discriminative profiles 60 is saved to allow extraction of data on methylation levels required to build appropriate equations.

Usage of Automated Tools for Cell or Tissue Origin and/or Type Prediction

Now the third component associated with the industrial applicability of the invention will be described in reference to FIG. 35-37, namely usage of automated tools (computer systems implementing programmable methods realizing) for sample characterizing/assessing, i.e. for classifying or typing or composition prediction of an unknown sample, called also a test sample. Automated tools should be understood as computer implemented methods performed by computer systems based on specific program instructions

In all methods of usage of said automated tools (as shown in FIG. 20), first a step of acquiring digital methylation data is required and then an appropriate methylation data pre-processing is needed for a test sample (or in general pre-processing of data relating to any measured epigenetic modification levels for said sample if the automated tools work with said any measured epigenetic modification levels). In practice digital data acquisition and preprocessing is the same as in the methods of generating data objects relating to known samples that were used for building appropriate automated tools.

For example, if the trained prediction model 70 is used for cell or type prediction contained in an unknown sample then digital data relating to said unknown sample has to be preprocessed so as to obtain a data object 50′ called CpG pair based discriminative profile 50′ of an unknow sample using the method as described in reference to FIG. 28.

In another embodiment, if the reference library 80 and tool for querying reference library 81 is used for cell or type prediction contained in an unknown sample then digital data relating to said unknown sample has to be preprocessed so as to obtain a data object 50′ called CpG pair based reference discriminative profile 50′ of an unknow sample using the method as described in reference to FIG. 28.

In another embodiment, if the composition prediction tool 90 (reference deconvolution model 90) is used for composition prediction in an unknown sample then digital data relating to said unknown sample also has to be preprocessed so as to obtain a data object 50′ called CpG pair based reference discriminative profile 50′ of an unknow sample using the method as described in reference to FIG. 28.

In particular, digital data objects constructed for said test sample have to have the same data volume and contain the same set of pairs of epigenetically modified bases as the digital objects for appropriate known samples, i.e. has to be generated for the same specific application context.

As shown in FIG. 35, a computer implemented method of classifying a tissue or cell of origin contained in a test sample, comprises: a) for said test sample providing a discriminative profile 50′ based on pairs of adjacent epigenetically modified bases, said profile 50′ for said test sample being received by the method described in reference to FIG. 28 and containing only pairs of adjacent epigenetically modified bases which were used to train the model 70, b) inputting said discriminative profile 50′ based on pairs of adjacent epigenetically modified bases for said test sample into a trained classification model 70, c) receiving a result of the classification 72 that indicates origin and/or type of the tissue or cell contained in said test sample

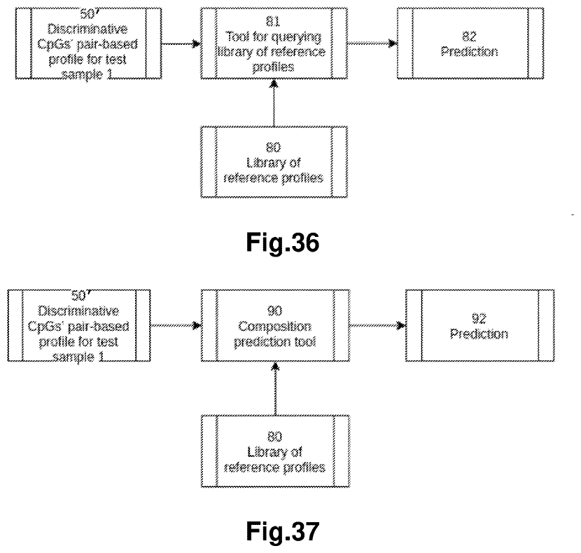

As shown in FIG. 36, a computer implemented method for determining a tissue or cell of unknown origin contained in a test sample comprises: providing a library of reference cell or tissue type discriminative profile based on pairs of adjacent epigenetically modified bases 80, the library being obtained by the method described in reference to FIG. 33; providing for said test sample a discriminative profile 50′ based on pairs of adjacent epigenetically modified bases, said profile 50′ being received by the method described in reference to FIG. 28 and containing only pairs of adjacent epigenetically modified bases which were used to appropriate reference discriminative profile 60 for known samples that were used to generate said library 80, querying said library 80 of reference discriminative profiles 60 based on pairs of adjacent epigenetically modified bases with said pre-processed profile 50′ and querying tool 81 of said tissue or cell of unknown origin contained in said sample; and receiving a result 82 of the query that indicates origin and/or type of the tissue or cell of contained in said test sample.

As shown in FIG. 37, a computer implemented method for predicting a sample composition of an unknown test sample, namely for tissue and/or cell proportion estimation in an unknown sample, comprises: providing a library 80 of reference discriminative profiles 60 based on pairs of adjacent epigenetically modified bases the library being obtained by the method described in reference to FIG. 33; providing for said test sample a discriminative profile 50′ based on pairs of adjacent epigenetically modified bases for said unknown test sample, said profile 50′ being received by the method described in reference to FIG. 28 and containing only pairs of adjacent epigenetically modified bases which were used to appropriate reference discriminative profile 60 for known samples that were used to generate said library 80, performing deconvolution of the composition of said unknown sample by a reference deconvolution model 90; and receiving a result 92 of the deconvolution that includes predicted sample composition.

As mentioned, automated tools according to the invention are used for determining the type or origin of samples. The methods according to the invention in which said automated tools are used have broad utility in diagnostics. In some embodiments, plurality of DNA samples can be taken from the same patient over a period of time. CpG pairs methylation maps and profiles derived from these samples can be used at all stages of clinical disease management, from assessment of risk and predisposition screening, through diagnosis, disease prognosis and management, to monitoring of the relapse. For example, assessment of these profiles may be particularly useful in certain conditions, as for example early detection of cancer, determining primary site of a tumor in case of cancers of unknown primary site (CUP), or monitoring the condition of the organ after transplantation.

In some embodiments, methods disclosed herein can be applied to analyze the composition and tissue origin of cfDNA samples. In some embodiments, changes in such compositions can be used to monitor the health of an individual. For example, detecting presence of cancerous nucleic acid material is an obvious warning sign, which warrants further tests and examinations. For example, the sudden decrease of a particular DNA component in the cfDNA sample of an individual may also suggest altered health conditions.

In some embodiments the third component of the industrial applicability of the present invention can be described as a general method of assessing correlation of a sample to be tested with cell or tissue type, the method comprising:
a) a differential methylation pairs DMP partitioning step of determining a plurality of target DMPs for evaluation based on specificity of methylation status in said DMPs in at least one sample type among at least one from another sample types, wherein said DMPs being pairs of adjacent CpG sites localized within one nucleic acid molecule;
b) cell or tissue type assessment step of assessing the correlation of the sample to be tested with the cell or tissue type based on the methylation status of the target DMPs of the sample to be tested.
   Said differential methylation pairs DMP partitioning step covers all methods and any of their details relating to generation of data objects based on pairs of adjacent CpGs and their appropriate steps as presented in FIGS. 21-22, 25-26 and 28-29, in particular it covers finding differential methylation pairs DMPs also called discriminative CpG pairs within a specific application context (as contained in discriminative maps or discriminative profiles).

Said cell or tissue type assessment step covers any possible details of any of methods relating to generation of automated tools for assessing tested sample as shown in FIG. 30-31, 33-34 and their usage for assessing tested samples as shown in FIG. 35-37.

Yet in some embodiments the third component of the industrial applicability of the present invention can be described as a general computer implemented method of characterizing a test s ample from a subject, comprising:
a) receiving methylation level data provided by measuring methylation level of consecutive nucleic acid sequence contained in said test sample from the subject;
b) providing for said test sample a CpG pair-based discriminative methylation profile based on methylation level data, wherein the CpG pair-based methylation discriminative profile comprises a differential methylation pairs (DMPs) selected based on specificity of methylation status in said DMPs in at least one sample type among at least one from another sample types, said profile containing genomic coordinates of said consecutive DMPs and a single value indicative of methylation status for said DMPs in said sample;
c) inputting said CpG pair based methylation profile to a sample characterizing tool, in order to determine sample type, said tool outputting the result of sample characterization and using for characterization one or more pre-established methylation signatures, wherein each of the one or more preestablished methylation signatures correlates with sample type, and wherein each pre-established methylation signature comprises a set of differential methylation pairs DMP selected based on specificity of methylation status in said DMPs in at least one sample type among at least one from another sample types; Said step of providing for said test sample a CpG pair-based methylation profile based on methylation level data covers any possible details of any of methods relating to generation of data objects based on pairs of adjacent CpGs and their appropriate steps as presented in FIGS. 21-22, 25-26 and 2829, in particular it covers finding differential methylation pairs DMPs also called discriminative CpG pairs within a specific application context (as contained in discriminative maps or discriminative profiles).

Said step of inputting said CpG pair based methylation profile to a sample characterizing tool covers usage of all methods and any of their details relating to generation of automated tools for assessing tested sample as shown in FIG. 30-31, 33-34 and their usage for assessing tested samples as shown in FIG. 35-37.

In particular, said sample characterizing tool is a software module comprising a model for cell or tissue type classifying being provided by the following steps:
a) providing a training data set by
   providing a first set of cell or tissue type discriminative profiles based on pairs of adjacent CpG sites within a specific application context for the first known type of the tissue or cell along with associated labels indicating the first known type of tissue or cell
   providing at least a second set of cell or tissue type discriminative profile based on pairs of adjacent CpG sites within a specific application context for at least the second known type of the tissue or cell along with associated labels indicating the at least second known type of tissue or cell;
b) training the classification model with said training data set so as it is configured to classify the type of a cell or tissue relating to an input cell or tissue type discriminative profile based on pairs of adjacent CpG sites, said tool being configured to output a result of the classification that indicates the tissue or cell of origin contained in said test sample.

In another embodiment the model is a reduced trained model for cell or tissue type classifying provided by further eliminating unnecessary pairs of adjacent CpG sites in the training data set by further training said classification model with the use of a feature selection method, then saving reduced discriminative map and saving said reduced trained model.

There is also provided for that purpose, a computing platform (computing system) for characterizing a test sample from a subject, comprising:
a computing device comprising a processor, a memory module, an operating system, and a computer program including instructions executable by the processor to create a sample characterizing application, the sample characterizing application comprising a data analysis module configured to:
a) receive methylation level data provided by measuring methylation level of consecutive nucleic acid sequence contained in said test sample from the subject;
b) provide for said test sample a CpG pair-based discriminative methylation profile based on methylation level data, wherein the CpG pair-based methylation discriminative profile comprises a differential methylation pairs (DMPs) selected based on specificity of methylation status in said DMPs in at least one sample type among at least one from another sample types, said profile containing genomic coordinates of said consecutive DMPs and a single value indicative of methylation status for said DMPs in said sample;
wherein said data analysis module further comprises a sample characterizing tool configured to determine sample type, said tool outputting the result of sample characterization and using for characterization one or more pre-established methylation signatures, wherein each of the one or more pre-established methylation signatures correlates with sample type, and wherein each pre-established methylation signature comprises a set of differential methylation pairs DMP selected based on specificity of methylation status in said DMPs in at least one sample type among at least one from another sample types;

Said sample characterizing tool comprised in the computing platform can be one among: a trained model for cell or tissue type classifying, a library of reference discriminative profiles based on DMPs along with a querying tool for typing cell or tissue contained in said test sample, a library of reference discriminative profiles based on DMPs along with a deconvolution tool for indicating composition of the test sample;

The computing platform can comprise other computing devices, for example the ones which are configured to support specific epigenetic modification level measurements.

Additionally, the present invention is useful in treating and/or diagnosing cancer or another pathological condition.

In one embodiment, the method for diagnosing a cancer in a patient comprises the following steps of:
a) identifying a plurality of adjacent CpG pairs-based features of a cancer type t, wherein the plurality of adjacent CpG pairs-based features has a total number K of adjacent CpG pairs-based features, K being a positive integer, said features being the methylation status in differential methylation pairs DMP selected based on specificity of methylation status in said DMPs in at least one sample type among at least one from another sample types;
b) providing digital data on values of methylation level measurements for CpG sites contained in nucleic acid in a test sample from a cell or a tissue, wherein providing said digital data includes:
   providing said sample from a subject,
   extracting nucleic acid fragments from said sample,
   converting said extracted nucleic acid fragments,
   assessing methylation levels in said converted isolated nucleic acid so as to receive a plurality of values of methylation level measurements for said sample thereby providing digital data on values of methylation level measurements for CpG sites.
c) generating a CpG pairs-based discriminative methylation profile based on said digital data of methylation level measurements for said test sample, wherein the CpG pair-based methylation discriminative profile comprises a differential methylation pairs (DMPs) selected based on specificity of methylation status in said DMPs in at least one sample type among at least one from another sample types, said profile containing genomic coordinates of said consecutive DMPs and a single value indicative of methylation status for said DMPs in said test sample;
d) determining that the subject has the cancer type t, when the calculated prediction score A being the result of the comparison of said CpG pairs-based discriminative methylation profile with said plurality of adjacent CpG pairs features of a cancer type t is greater than a pre-determined threshold.
   Embodiments concern a patient who has symptoms of cancer, is asymptomatic of cancer, has a family or patient history of cancer, is at risk for cancer, or who has been diagnosed with cancer. A patient may be a mammalian patient though in most embodiments the patient is a human. The cancer may be malignant, benign, metastatic, or a precancer.

In some embodiments, the cancer tissue is selected from a group consisting of liver cancer tissue, lung cancer tissue, kidney cancer tissue, colon cancer tissue, brain cancer tissue, pancreas cancer tissue, brain cancer tissue, gastrointestinal cancer tissue, head and neck cancer tissue, bone cancer tissue, tongue cancer tissue, gum cancer tissue, and combinations thereof. In other embodiments, the tissue is selected from a group consisting of liver tissue, brain tissue, lung tissue, kidney tissue, colon tissue, pancreas tissue, brain tissue, gastrointestinal tissue, head and neck tissue, bone, tongue tissue, gum tissue, and combinations thereof.

In some embodiments, the sample is a biological sample, selected from the group consisting of diseased tissue, cancer tissue, tissue from a specific organ, liver tissue, lung tissue, kidney tissue, colon tissue, T-cells, B-cells, neutrophils, small intestines tissue, pancreas tissue, adrenal glands tissue, esophagus tissue, adipose tissue, heart tissue, brain tissue, placenta tissue, and combinations thereof. In some embodiments, the patient may be diagnosed to have a disease or condition or be diagnosed specifically not to have the disease or condition.

In some embodiments, the sample is a cfDNA sample, prepared from a plasma sample obtained from blood sample of the subject. The biological sample may be any biological liquid such as saliva, amniotic fluid, cystic fluid, spinal or brain fluid, urine, sweat, or tears.

In one embodiment the method for treating a subject with a cancer comprises the following steps of: a) identifying a plurality of adjacent CpG pairs-based features of a cancer type t, wherein the plurality of adjacent CpG pairs-based features has a total number K of adjacent CpG pairs-based features, K being a positive integer, said features being the methylation status in differential methylation pairs DMP selected based on specificity of methylation status in said DMPs in at least one sample type among at least one from another sample types;
b) generating a CpG pairs-based discriminative methylation profile, for a cell or tissue of the subject contained in a test sample, wherein the CpG pair-based methylation discriminative profile comprises a differential methylation pairs (DMPs) selected based on specificity of methylation status in said DMPs in at least one sample type among at least one from another sample types, said profile containing genomic coordinates of said consecutive DMPs and a single value indicative of methylation status for said DMPs in said test sample;
c) determining that the subject has the cancer type t, when the calculated prediction score AA being the result of the comparison of said CpG pairs-based methylation profile with said plurality of adjacent CpG pairs features of a cancer type t is greater than a pre-determined threshold; and
d) administering a treatment to the subject based on the determining that the subject has the cancer type t, wherein the treatment comprises a member selected from the group consisting of a chemotherapy, a radiation therapy, an immunotherapy, and a tumor resection.

Advantageously, the step b) comprises
A1) for said test sample relating to a certain type of tissue or cell providing its profile based on pairs of adjacent CpG sites,
B1) providing a discriminative map of cells or tissues types based on pairs of adjacent CpG sites, said map being generated for said specific application context and containing only differential methylation pairs (DMPs) selected based on specificity of methylation status in said DMPs in at least one known sample type among at least one from another known sample types,
C1) saving, only for pairs of adjacent CpG sites contained in said discriminative map of cell or tissues types,
   genomic coordinates of adjacent CpG sites and
   compressed methylation information in the form of one single numerical value relating to said adjacent CpG sites,
so as to generate said cell or tissue type discriminative profile based on pairs of adjacent CpG sites for said sample relating to a certain type of tissue or cell within said specific application context.

Advantageously, the step A1) comprises
i) determining a set of pairs of adjacent CpG sites in said nucleic acid contained in said test sample, each pair of adjacent CpG sites consisting of two adjacent CpG sites localized within one nucleic acid molecule so as to generate a map of adjacent CpG sites containing genomic coordinates;
ii) compressing information on methylation level value, iteratively for each pair of adjacent CpG sites among the determined set of pairs of adjacent CpG sites, by converting two values of methylation level associated with two CpG sites in each pair, into one single numerical value, said single value representing compressed methylation information for a pair of adjacent CpG sites and being indicative of methylation status in each pair of adjacent CpG sites;
iii) saving
   genomic coordinates of adjacent CpG sites for each pair in said determined set of pairs and
   compressed methylation information in the form of one single numerical value for said each pair of adjacent CpG sites in said sample
so as to generate a profile based on pairs of adjacent CpG sites of said tissue or said cell.

Advantageously, the methylation status is one among at least co-methylation status and non co-methylation status in a pair of adjacent CpG sites and wherein converting two values of methylation level associated with two CpG sites in each pair, into one single numerical value involves calculating methylation value difference between two CpG sites in each pair of said adjacent CpG sites.

Advantageously, the step B1) comprises
i) providing at least two parametrized profiles based on pairs of adjacent CpG sites for at least two different known types of cells or tissues generated with the method according to any of claim 10 to 18, wherein the number and the known types of cells or tissues, for which the parametrized profiles are provided, giving a specific application context.
ii) determining and comparing the methylation status contained in the model parameters for each pair of adjacent CpG sites present in all of said at least two parametrized profiles
iii) extracting each pair to be a part of said discriminative map of cells or tissues types under construction if the methylation status in at least one parametrized profile for said pair is different from the methylation status in at least one among all other parametrized profiles.
iiii) saving genomic coordinates for extracted pairs of adjacent CpG sites so as to generate said discriminative map of cells or tissues types for a specific application context, said extracted pairs being differential methylation pairs (DMPs) selected based on specificity of methylation status in said DMPs in at least one known sample type among at least one from another known sample types.

Advantageously, the step v) of constructing parametrized profile based on pairs of adjacent CpG sites of a known type of tissue or cell based on methylation information derived from nucleic acid contained in a group of at least one sample, each sample relating to the same known type of said tissue or said cell, comprises:
a) for each sample in said group of at least one sample providing digital data on methylation level measurements for CpG sites contained in said nucleic acid in said sample relating to said tissue or said cell of a known type
b) determining a set of pairs of adjacent CpG sites in said nucleic acid contained in each sample in said group of at least one sample relating to said tissue or said cell of a known type, each pair of adjacent CpG sites consisting of two adjacent CpG sites localized within one nucleic acid molecule so as to generate a map of adjacent CpG sites containing genomic coordinates; then
c) for said group of at least one sample iteratively for each pair of adjacent CpG sites among the determined set of pairs of adjacent CpG sites, compressing information on methylation level value, by fitting a parametrized mathematical model to said digital data on values of methylation level measurements associated with each pair of adjacent CpG sites so as to determine one single model parameter, said single model parameter representing compressed methylation information for a pair of adjacent CpG sites and being indicative of methylation status in each pair of adjacent CpG sites for said group of at least one sample;
d) for said group of at least one sample saving
   genomic coordinates of adjacent CpG sites for each pair in said determined set of pairs and
   compressed methylation information in the form of one model parameter for said each pair of adjacent CpG sites in said group of at least one sample
   so as to generate said parametrized profile based on pairs of adjacent CpG sites of a known type of tissue or cell.

Advantageously, the prediction score A can be outputted by different tools, namely by 1) a machine learning model for cell or tissue type classifying, 2) a library of reference discriminative profiles based on DMPs along with a querying tool for typing cell or tissue contained in said test sample, 3) a library of reference discriminative profiles based on DMPs along with a deconvolution tool for indicating composition of the test sample and wherein the cancer type to be determined comprising one among: acute myeloid leukemia (LAML or AML), acute lymphoblastic leukemia (ALL), adrenocortical carcinoma (ACC), bladder urothelial cancer (BLCA), brain stem glioma, brain lower grade glioma (LGG), brain tumor, breast cancer (BRCA), bronchial tumors, Burkitt lymphoma, cancer of unknown primary site, carcinoid tumor, carcinoma of unknown primary site, central nervous system atypical teratoid/rhabdoid tumor, central nervous system embryonal tumors, cervical squamous cell carcinoma, endocervical adenocarcinoma (CESC) cancer, childhood cancers, cholangiocarcinoma (CHOL), chordoma, chronic lymphocytic leukemia, chronic myelogenous leukemia, chronic myeloproliferative disorders, colon (adenocarcinoma) cancer (COAD), colorectal cancer, craniopharyngioma, cutaneous T-cell lymphoma, endocrine pancreas islet cell tumors, endometrial cancer, ependymoblastoma, ependymoma, esophageal cancer (ESCA), esthesioneuroblastoma, Ewing sarcoma, extracranial germ cell tumor, extragonadal germ cell tumor, extrahepatic bile duct cancer, gallbladder cancer, gastric (stomach) cancer, gastrointestinal carcinoid tumor, gastrointestinal stromal cell tumor, gastrointestinal stromal tumor (GIST), gestational trophoblastic tumor, glioblastoma multiforme glioma GBM), hairy cell leukemia, head and neck cancer (HNSD), heart cancer, Hodgkin lymphoma, hypopharyngeal cancer, intraocular melanoma, islet cell tumors, Kaposi sarcoma, kidney cancer, Langerhans cell histiocytosis, laryngeal cancer, lip cancer, liver cancer, Lymphoid Neoplasm Diffuse Large B-cell Lymphoma [DLBCL), malignant fibrous histiocytoma bone cancer, medulloblastoma, medullo epithelioma, melanoma, Merkel cell carcinoma, Merkel cell skin carcinoma, mesothelioma (MESO), metastatic squamous neck cancer with occult primary, mouth cancer, multiple endocrine neoplasia syndromes, multiple myeloma, multiple myeloma/plasma cell neoplasm, mycosis fungoides, myelodysplastic syndromes, myeloproliferative neoplasms, nasal cavity cancer, nasopharyngeal cancer, neuroblastoma, Non-Hodgkin lymphoma, nonmelanoma skin cancer, non-small cell lung cancer, oral cancer, oral cavity cancer, oropharyngeal cancer, osteosarcoma, other brain and spinal cord tumors, ovarian cancer, ovarian epithelial cancer, ovarian germ cell tumor, ovarian low malignant potential tumor, pancreatic cancer, papillomatosis, paranasal sinus cancer, parathyroid cancer, pelvic cancer, penile cancer, pharyngeal cancer, pheochromocytoma and paraganglioma (PCPG), pineal parenchymal tumors of intermediate differentiation, pineoblastoma, pituitary tumor, plasma cell neoplasm/multiple myeloma, pleuropulmonary blastoma, primary central nervous system (CNS) lymphoma, primary hepatocellular liver cancer, prostate cancer such as prostate adenocarcinoma (PRAD), rectal cancer, renal cancer, renal cell (kidney) cancer, renal cell cancer, respiratory tract cancer, retinoblastoma, rhabdomyosarcoma, salivary gland cancer, sarcoma (SARC), Sezary syndrome, skin cutaneous melanoma (SKCM), small cell lung cancer, small intestine cancer, soft tissue sarcoma, squamous cell carcinoma, squamous neck cancer, stomach (gastric) cancer, supratentorial primitive neuroectodermal tumors, T-cell lymphoma, testicular cancer testicular germ cell tumors (TGCT), throat cancer, thymic carcinoma, thymoma (THYM), thyroid cancer (THCA), transitional cell cancer, transitional cell cancer of the renal pelvis and ureter, trophoblastic tumor, ureter cancer, urethral cancer, uterine cancer, uterine cancer, uveal melanoma (UVM), vaginal cancer, vulvar cancer, Waldenstrom macroglobulinemia, or Wilm's tumor.

Advantageously, a chemotherapy comprises administrating one among: alkylating agents, preferably bifunctional alkylators, monofunctional, anthracyclines, epothilones; histone deacetylase, topoisomerase I inhibitors, topoisomerase II inhibitors, kinase inhibitors, nucleotide analogs and nucleotide precursor analogs, peptide antibiotics, platinum-based antineoplastics, retinoids, and vinca alkaloids, and wherein a immunotherapy comprises administrating one among: cellular therapy, preferably dendritic cell therapy, antibody therapy, and cytokine therapy.

In further embodiments the above-mentioned method covers methods for treating cancer in a cancer patient comprising administering to the patient an effective amount of chemotherapy, radiation therapy, or immunotherapy (or a combination thereof) after the patient has been diagnosed to have cancer based on methods disclosed herein. The point of origin of the cancer may be determined, in which case, the treatment is tailored to cancer of that origin. In some embodiments, tumor resection is performed as the treatment or may be part of the treatment with one of the other treatments. Examples of chemotherapeutics include, but are not limited to, the following: alkylating agents such as bifunctional alkylators (for example, cyclophosphamide, mechlorethamine, chlorambucil, melphalan) or monofunctional alkylators (for example, dacarbazine (DTIC), nitrosoureas, temozolomide (oral dacarbazine)); anthracyclines (for example, daunorubicin, doxorubicin, epirubicin, idarubicin, mitoxantrone, valrubicin; taxanes, which disrupt the cytoskeleton (for example, paclitaxel, docetaxel, abraxane, taxotere); epothilones; histone deacetylase inhibitors (for example, vorinostat, romidepsin); Topoisomerase I inhibitors (for example, irinotecan, topotecan); Topoisomerase II inhibitors (for example, etoposide, teniposide, tafluposide); kinase inhibitors (for example, bortezomib, erlotinib, gefitinib, imatinib, vemurafenib, vismodegib); nucleotide analogs and nucleotide precursor analogs (for example, azacitidine. azathioprine, capecitabine, cytarabine, doxifluridine. fluorouracil, gemcitabine, hydroxyurea, mercaptopurine, methotrexate, tioguanine (formerly thioguanine); peptide antibiotics (for examples, bleomycin, actinomycin); platinum-based antineoplastics (for example, carboplatin, cisplatin, oxaliplatin); retinoids (for example, retinoin, alitretinoin, bexarotene); and, vinca alkaloids (for example, vinblastine, vincristine, vindesine, vinorelbine). Immunotherapies include, but are not limited to, cellular therapy such as dendritic cell therapy (for example, involving chimeric antigen receptor); antibody therapy (for example, Alemtuzumab, Atezolizumab, Ipilimumab, Nivolumab, Ofatumumab, Pembrolizumab, Rituximab or other antibodies with the same target as one of these antibodies, such as CTLA-4, PD-1, PD-L1, or other checkpoint inhibitors); and, cytokine therapy (for example, interferon or interleukin).

In certain embodiments, there are methods of diagnosing a patient based on determining whether the patient has a CpG pairs-based methylation profile indicative of cancer or another disease or condition. In some embodiments, methods involve generating a CpG pairs based methylation profile that indicates whether the patient has cancer or another disease or condition, and if so, from what organ.

In certain embodiments, this is done using a biological sample from the patient that comprises cell free DNA.

### Computer System and Computer Readable Medium

An exemplary system configured to perform all the methods according to the invention is shown in FIG. 38.

The system comprises an example computer system 100 for implementing the entities shown in FIG. 20-22,25-26, 28-31,33-37. The computer system 100 includes a central processing unit (CPU, also “processor” and “computer processor” herein) 102, which can be a single core or multi core processor, either through sequential processing or parallel processing. The computer system 100 also includes a memory unit or device 106 (e.g., random-access memory, read-only memory, flash memory), a storage unit or device 109 (e.g., hard disk), a communication interface 111 (e.g., network adapter) for communicating with one or more other systems, and peripheral devices, either external or internal or both, such as a printer, monitor, USB drive and/or CD-ROM drive. It also comprises the touch-screen interface, a mouse, track ball, or other type of pointing device, a keyboard, or some combination thereof, and is used to input data into the computer system 100. The memory 106, storage unit 109, communication interface 111 and peripheral devices 104 are in communication with the CPU 102 through a communication bus (solid lines), such as a motherboard. The storage unit 109 can be a data storage unit (or data repository) for storing data. The computer system 100 can be operatively coupled to a computer network (“network”) 101 with the aid of the communication interface 111. The network 101 can be the Internet, an internet and/or extranet, or an intranet and/or extranet that is in communication with the Internet. The network 101 in some cases is a telecommunication and/or data network. The network 101 can include one or more computer servers, which can enable a peer-to-peer network that supports distributed computing. The network 101, in some cases with the aid of the computer system 100, can implement a client-server structure, which may enable devices coupled to the computer system 100 to behave as a client or a server. Other embodiments of the computer system 100 have different architectures.

The storage device 109 is a non-transitory computer-readable storage medium such as a hard drive, compact disk read-only memory (CD-ROM), DVD, or a solid-state memory device. The memory 106 holds instructions and data used by the processor (CPU) 102.

The computer system 100 can include or be in communication with an electronic display 108 that comprises a user interface (UI) 110. Examples of UI's include, without limitation, a graphical user interface (GUI) and web-based user interface. The graphics adapter (not shown) displays images and other information on the display 108. The network adapter (communication interface) 111 couples the computer system 100 to one or more computer networks. The network 101 can be the Internet, an internet and/or extranet, or an intranet and/or extranet that is in communication with the Internet. The network 101 in some cases is a telecommunication and/or data network. The network 101 can include one or more computer servers, which can enable a peer-to-peer network that supports distributed computing. The network, in some cases with the aid of the computer system, can implement a client-server structure, which may enable devices coupled to the computer system to behave as a client or a server.

The computer system 100 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 109, loaded into the memory 106, and executed by the processor 102.

Types of computer systems 100 (shown in FIG. 38) used by the entities of FIG. 33-37 can vary depending upon the embodiment and the processing power required by the entity. For example, the classification model unit can run in a single computer 100 or multiple computers 100 communicating with each other through a network such as in a server farm. The computer system 100 can regulate various aspects of the present disclosure, such as, for example, inputting amino acid position information, transferring imputed information into datasets, and generating a trained algorithm with the datasets. The computer system 100 can be a user electronic device or a remote computer system. The electronic device can be a mobile electronic device. The computer system 100 can lack some of the components described above, such as graphics adapters, and displays 108.

Provided herein is also a non-transitory computer readable medium comprising machine-executable code that, upon execution by one or more computer processors, implements any of methods according to the invention.

The code can be pre-compiled and configured for use with a machine having a processor adapted to execute the code, or it can be compiled during runtime. The code can be supplied in a programming language that can be selected to enable the code to execute in a pre-compiled or as compiled fashion.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as the main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### EXAMPLES

Now examples of generation and use of automated tools for sample characterizing(assessing) according to the invention will be described, including examples of certain data objects like reference discriminative profiles 50.

### Example 1

The first example relates to establishment and use of library of reference discriminative profiles 80. An exemplary library of reference discriminative profiles 80 was generated for a dataset containing three groups of samples for three different types of lung cells respectively: 288 lung adenomas and adenocarcinomas tumour samples, 235 lung squamous cell neoplasms tumour samples and 57 healthy lung samples.

Dataset was randomly split into training (70%) and test (30%) sets using stratified sampling.

For each group among said 3 training groups of samples a parametrized CpG pairs-based profile 30 was generated. It contains 28.826 CpG pairs.

Then the discriminative map 40 for said set of three types of lung cells was obtained and contained 58 pairs (beta>0.15, p-value<=0.05). In the next step, knowing said discriminative map 40, the CpG pairs-based profiles 20 of all samples were processed to obtain discriminative profiles 50.

Then, for each group of samples, data from said discriminative profiles 50 were averaged to generate reference profiles 60 for the lung adenomas and adenocarcinomas tumour, lung squamous cell neoplasms tumour and healthy lung.

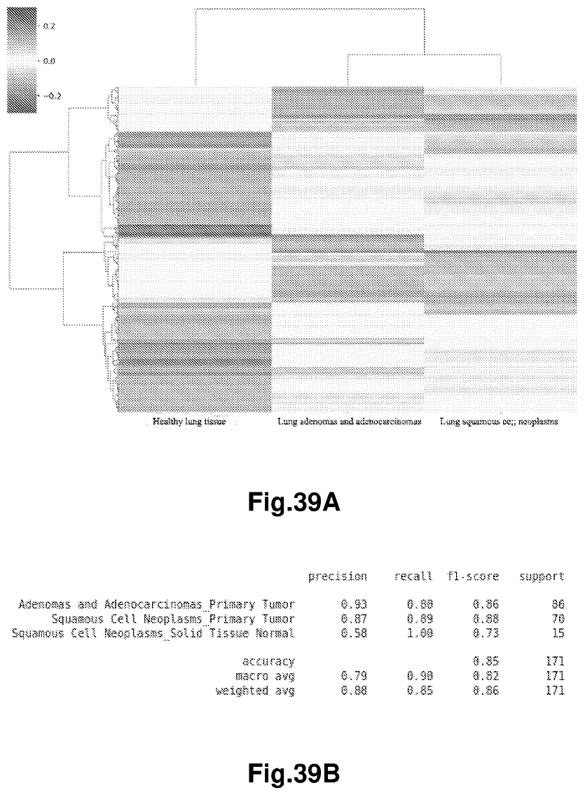

The reference library 80 of reference discriminative profiles 60 for the three different types of lung cells generated according to the invention, containing 58 CpG pairs, non-co-methylated in at least one group of samples [beta>=0.15, p-value=0.05](FIG. 39a)

For each sample in the test set, prediction was performed using library 80 of reference discriminative profiles 60 and standardized Euclidean distance was calculated by the querying tool 81 according to the pipeline as shown in FIG. 36. The performance metrics of said searchable library for which reference discriminative profiles 60 containing only 58 CpG pairs were used are discussed in FIG. 39b.

### Example 2

The second example relates to check of resistance of methylation data processing according to the invention to the batch effect. The batch effect is a technical variance (bias) observed across different measurement series/runs/slides and caused by different technical conditions of measurements. Bias may have different forms for instance it may falsely increase or decrease measured methylation levels making experiments not comparable or significantly biasing the comparison towards one or more data sets in the comparison, what may lead to false experimental conclusions.

An experimental check has been performed for particular measurement technology, namely for Ilumina Infinium MethylationEPIC BeadChip. The person skilled in the art will know that Ilumina slide is designated to analyze 8 samples in one run, namely one slide contains 8 microarrays, each allowing to perform measurements for one sample. For the purpose of said experiment identical technical conditions for measurements using this technology were used, the samples were run on different slides. It should be noted that the use of different measurements instruments means use of two copies of the same type of measurement instruments, which results in using two devices with the same technical specifications as supplied by the manufacturer. The person skilled in the art will know that despite the use of the instruments with identical specifications and microarrays produced with the same specification and complying provided by the company quality assessment differences between measurements may occur mainly due to for example:
sample quality
sample preservation and shipment
nucleic acid isolation technique
wash/clean-up conditions
ambient conditions, including room temperature and ozone levels differences in scanner/other hardware
   (Ross J P, van Dijk S, Phang M, Skilton M R, Molloy P L, Oytam Y. Batch-effect detection, correction and characterisation in Illumina HumanMethylation450 and MethylationEPIC BeadChip array data)

The following hypothesis should be verified: considering example of 16 samples measured using 2 slides (2 slides*8 samples=16 methylation profiles) if batch effect occurs, it will affect all CpG sites on the slide including first and successive CpGs of all pairs equally by increasing or decreasing the methylation level measured for each CpG site on one slide.

In this example, if the batch effect increases or decreases the methylation levels in one of the slides, the methylation levels measured for CpG sites on that slide will not be reflective of real methylation levels measured on the second slide where batch effect does not affect the methylation measurements.

However, the batch effect should not affect the comparison of delta methylation levels (difference of methylation levels in pairs of adjacent CpGs).

To further illustrate this example, two exemplary CpG sites can be assessed for which methylation values measured in first slide for first sample are as follows:

$$CpG 1 = 0.5$$

$$CpG 2 = 0.5$$

while the methylation level measured in the second slide (affected by batch effect resulting in an increase in the methylation level by 0.05 in this specific example) for the same first sample can be:

CpG1=0.5+0.05 (affected by batch-effect)

CpG2=0.5+0.05 (affected by batch-effect)

And for which the delta value calculated for CpG pair comprising CpG1 and CpG2 between slides should be equal because the batch effect is propagated uniformly across the whole slide.

$$First slide : \vert Delta \vert = CpG 2 - CpG 1 = 0.5 - 0.5 = 0$$

$$Second slide : \vert Delta \vert = CpG 2 ( affected by batch - effect ) 0.55 - CpG 1 ( affected by batch - effect ) 0.55 = 0$$

Proof of concepts involved performing own measurements and appropriate analysis. EPIC data for 69 blood samples collected from healthy women (GSE123914) were analyzed. Only 56/69 samples were used in this study. 13 samples were removed because the slides were not fully filled. Methylation levels were measured using 7 slides (8 samples per one slide). Methylation data have been processed so as to get CpG pairs-based profiles 20 according to the invention.

Due to the fact that all samples in this analysis were healthy woman in similar age it has been assumed that methylation levels of CpG sites between slides should not differ.

According to the experimental procedure there should be no methylation level differences between slides and the presence of the differences can only be attributed to batch effect. If methylation level differences or deltas of methylation levels (difference of methylation values in pairs of adjacent CpG sites) calculated based on measurements between slides are observed, those changes can only be attributed to batch effect.

To illustrate this principle, for each first CpG (n=96765) from each CpG pairs (n=96765) Kruskal-Walli's test was used to compare methylation levels across all slides. Next, for each CpG pair (n=96765) Kruskal-Walli's test was used to compare delta methylation levels (difference of methylation values in pairs of adjacent CpG sites) across all slides. All statistically significant results (FDR corrected p-value<=0.05) were considered as false positive and attributed to batch effect.

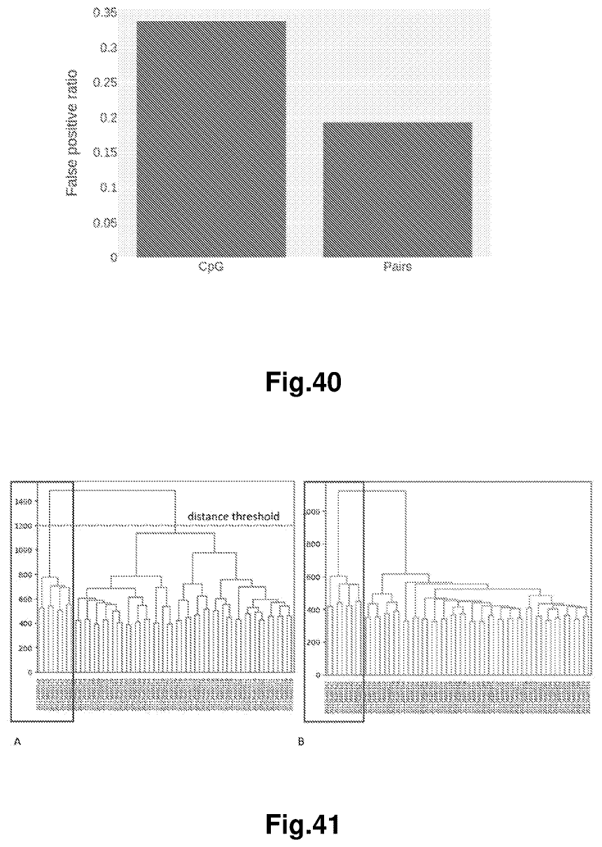

As shown in FIG. 40 false positive differences in methylation levels across at least one slide for 34% of analysed CpG sites (referred as false positive ratio) was observed.

In case of delta methylation levels measurements, the number of false positive differences in delta methylation levels (difference of methylation values in pairs of adjacent CpG sites) between slides was significantly reduced to 19%. As it can be seen in FIG. 40 the methylation signatures based on calculated delta methylation levels in pairs are more homogenous across slides, thus the batch effect is reduced to a significant extent.

Then, as a second proof of concept, cluster analysis was performed (dataset was the same as in the first analysis), using Ward method, Euclidian distance metric and scaled values for two datasets independently:
a) dataset containing methylation levels measurements for first CpG from each CpG-pair (56 samples, 96765 CpGs),
b) dataset containing delta values measurements for each CpG pair (56 samples, 96765 pairs).

Due to the fact that all samples in this analysis were healthy woman in similar age the methylation levels of CpG sites between slides should not differ.

If batch effect occurs for one or more slides and affects methylation levels for that slide or slides it is assumed that affected slide or slides should form separated cluster or clusters for specific preselected distance threshold in which the samples from the slides affected by batch effect will group.

As it can be seen from FIG. 41, part A for the dataset contains methylation levels for single CpG sites relating to several samples measured on one slide (id: 201236480142). Due to the fact that said set was affected by the batch effect, for a preselected (fixed) distance threshold two clusters of samples across different slides were identified.

On the other hand, for the difference value of methylation levels (for CpG pairs-based profiles) such two clusters were not observable for the same fixed threshold (see FIG. 41 part B).

Clustering results clearly indicate that methylation level measurements after conversion to delta methylation levels are more homogenous across samples from different slides what results in reduced number of clusters for specific distance threshold, and therefore reduced batch effect.

From the above, one can say that the present invention provides a universal and variables independent method, that overcomes technical variance (bias) observed across different measurement series/runs/slides, namely batch effect.

### Example 3

The third example relates to comparison of complexity of methylation signatures according to the invention with known priori art methylation signatures. Many authors published different types of classifiers using thousands of biomarkers, each biomarker being understood as a single CpG site.

It means that those models:
a) need large amount of data to measure for the model training purposes
b) need large amount of data to measure to make prediction
c) and because of both above mentioned, they are more expensive and prone to over-fitting.

Example number of biomarkers used in similar purposes are:
A) 2978 methylation biomarkers used for cancer type prediction from our study with the published studies predicting tumor origin based on CpG biomarkers [Modhukur V, Sharma S, Mondal M, Lawarde A, Kask K, Sharma R, Salumets A. Machine Learning Approaches to Classify Primary and Metastatic Cancers Using Tissue of Origin-Based DNA Methylation Profiles]
B) A total of 20,451 CpGs were used for predicting the origin tissues based on tumor cell lines by Zhang et al. [Zhang S, Zeng T, Hu B, Zhang Y H, Feng K, Chen L, Niu Z, Li J, Huang T, Cai Y D. Discriminating Origin Tissues of Tumor Cell Lines by Methylation Signatures and Dys-Methylated Rules]
C) 5709 CpGs from Tang et al.'s study [Tang W, Wan S, Yang Z, Teschendorff A E, Zou Q. Tumor origin detection with tissue-specific miRNA and DNA methylation markers], which discriminates tumor origin using a machine learning approach.
D) 10,360 CpGs from the work of Zheng et al. [Zheng C, Xu R. Predicting cancer origins with a DNA methylation-based deep neural network model]”

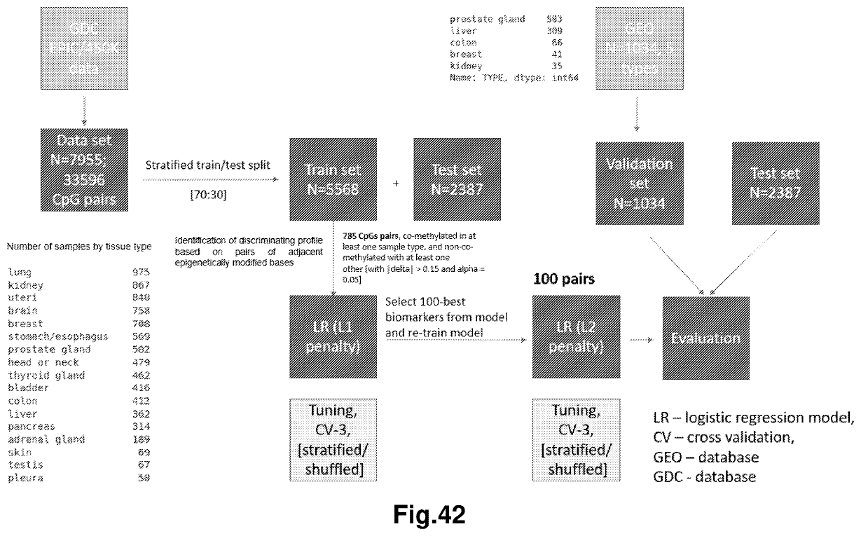

To enable the comparison of methylation signature complexity, the Authors of the present invention generated several tissue or cell specific CpG pair-based discriminative profiles using methods according to the invention described above and then performed two step training of the classification model in order to significantly reduce the number of methylation biomarkers. The whole exemplary process contained the steps as follows: 1. collecting 7955 samples from GDC database; 2. splitting into train [70%] and test [30%] set; 3. extracting cancer types discriminating profile 50 based on pairs of adjacent epigenetically modified bases for each sample (containing 785 CpG pairs if beta>0.15 and alpha=0.05); 4 training logistic regression model 70; 5. extracting top 100 biomarkers from the model; 6. re-training the model 70′ using only 100-top biomarkers; 7. evaluating model classification ability using test set; 8. collecting validation data from different database GEO (1034 samples); 9. evaluating model 70′ classification ability using validation set. The whole workflow can be seen in FIG. 42.

As it can be seen, even the first data objects received by the methods according to the invention, namely discriminative CpG pairs-based profiles 50 before reduction process, contained much less CpG sites than any known methylation signatures, i.e about 1600 CpG sites (see 785 CpG pairs). Moreover, the experiment showed that it is possible to select much less CpG sites, namely even only 200 CpG (100 CpG pairs) which were still discriminative and allowed to train an efficient classifier 70′.

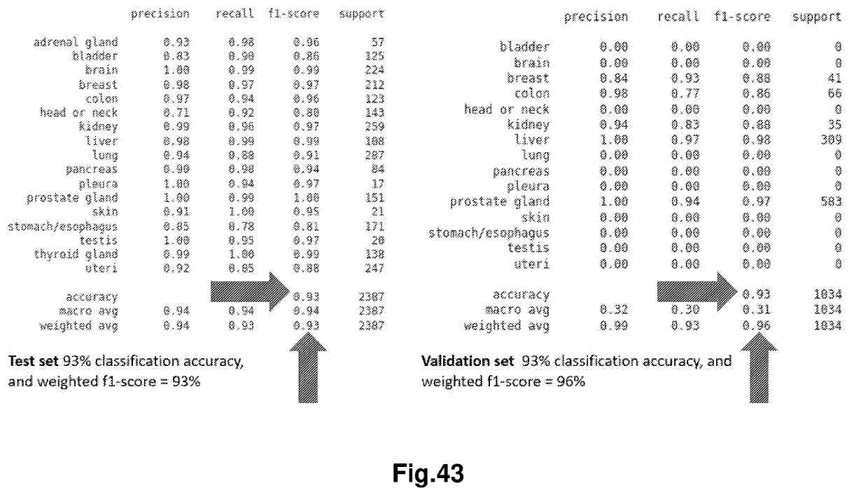

The performance metrics of the received model 70′ for which discriminative profiles 50 containing only 100 CpG pairs were used are discussed in FIG. 43. Thus, the method according to the present invention allows significant reduction of the number of markers necessary for tissue or cancer type prediction while preserving high accuracy of prediction, while existing models trained for similar

purposes use ~3000-30.000 biomarkers (single CpG sites). The use of pairs instead of single CpGs allows training machine learning models with reduced amount of data, namely with the use of minimum number of biomarkers, and in an efficient manner, namely with a high predictive ability.

### Example 4

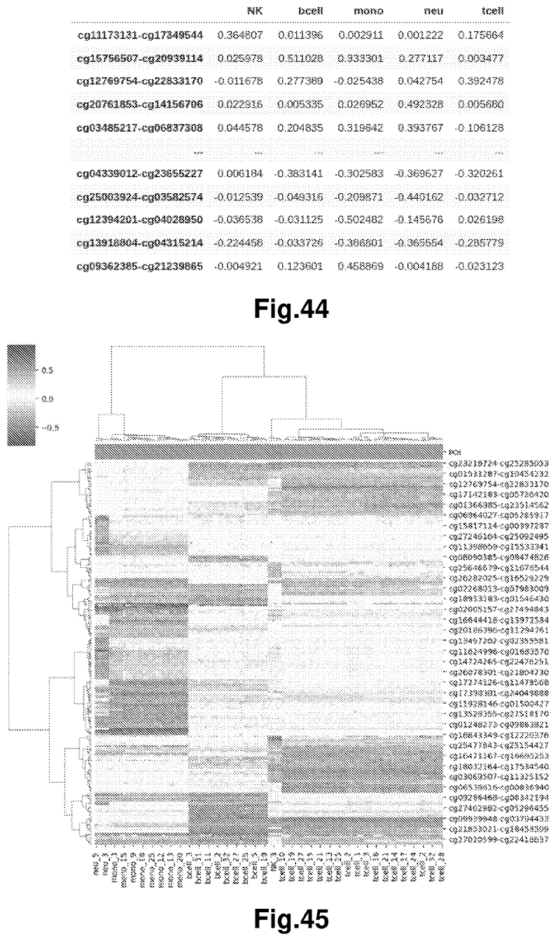

Another example relates to comparison of performance of estimation of cell types proportion (deconvolution) in biological samples according to the invention with known priori art estimation models. For that purpose, an exemplary reference atlas 80 containing reference discriminative profiles 60 according to the invention was built. It contained 5 reference profiles 60 for 5 main blood cell types, namely B-cell, T-cell, monocytes and neutrophiles (see FIG. 44 for specific delta methylation level values in exemplary CpG pairs)

All 5 profiles contained the same number of pairs, i.e. 220 pairs, and their visualization can be seen in FIG. 45.

To estimate sample composition, 3 state-of-the-art reference-based algorithms implemented in EpiDISH R package (Teschendorff A E, Breeze C E, Zheng S C, Beck S. A comparison of reference-based algorithms for correcting cell-type heterogeneity in Epigenome-Wide Association Studies) were used as an automated composition typing tool 90:
robust partial correlations (RPC)
Constrained Projection (CP)
Support Vector Regressions (CBS)
   As a reference the following reference atlases were used:
Default reference atlas—improved whole blood reference DNA methylation dataset from EpiDISH containing 333 CpG for 7 blood cell types (Teschendorff A E, Breeze C E, Zheng S C, Beck S. A comparison of reference-based algorithms for correcting cell-type heterogeneity in Epigenome-Wide Association Studies).
Custom reference atlas 80.

For 6 samples with known cell proportions (GSE112618), effectiveness of deconvolution algorithms used in the automated composition typing tool 90 with respect to different reference atlases were compared (default reference atlas implemented in EpiDISH package and custom CpG-pairs based reference atlas). Then for each sample and each cell type residuals, namely differences between estimated and expected cell proportion were calculated. Next for each sample median of absolute residuals as a final metric of estimation error (low metric=low estimation error) was calculated.

It should be noted that for 6 samples, real cell proportions (CD4T, CD8T, NK, B-cell, Monocytes, Granulocytes, Neutrophils) was calculated using state-of-the-art FACS (Fluorescence-activated cell sorting) method. Because only 5 cell types were analyzed by the Authors: t-Cell (CD4T+CD8T), NK, B-cell, Monocytes, Neutrophils, all real proportions per each sample were rescaled to sum to 1. It was necessary for said comparison, because all deconvolution algorithms assume that all proportions per sample should sum to 1. Moreover, deconvolution algorithms with default reference atlases estimate proportions also for CD4T and CD8T cell types, but in said comparison, those values were summed to general t-cell category.

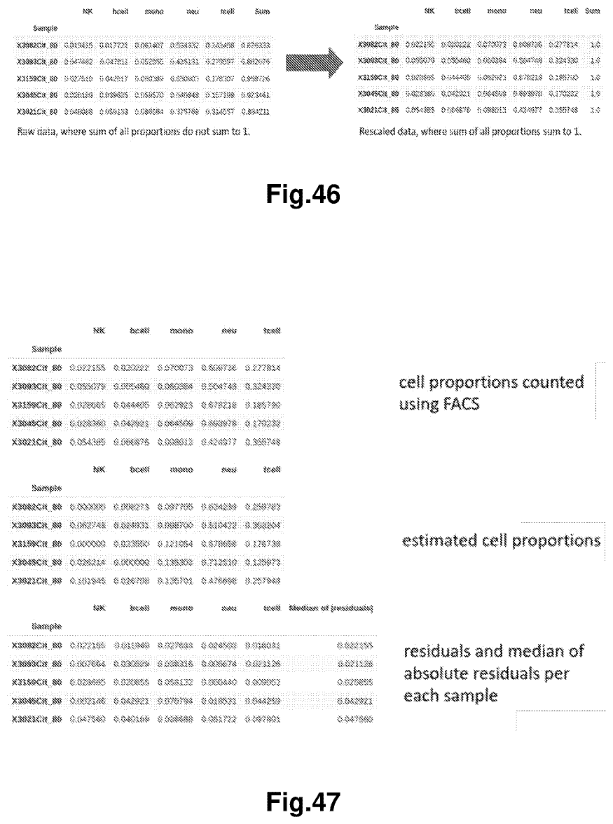

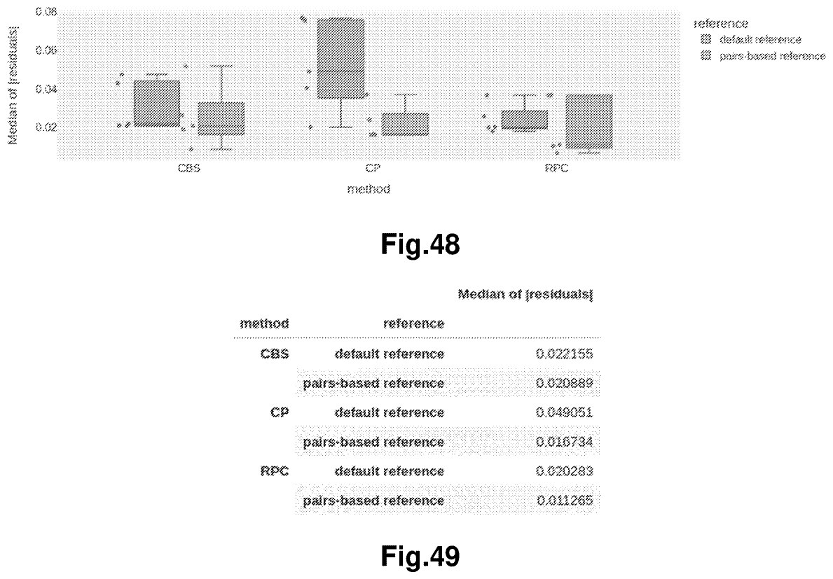

Such rescaling can be seen in FIG. 46 while calculation of median of absolute residuals can be seen in FIG. 47. Conclusions from the comparison are as follows: in case of all tested algorithms CpG pairs-based profiles 60 allows more precise cell proportion estimation (deconvolution). As shown in FIG. 48 illustrating a plot of estimation residuals, the residuals (errors) are significantly smaller using CpG pairs-based reference atlas 80 in comparison with default reference atlas 80 for all three different deconvolution algorithms used in the automated composition typing tool 90 according to the invention.

Median of residuals in numbers can be seen in FIG. 49.

### Example 5

The fifth example relates to CpG pairs-based reference library 80 (atlas) used with the automated tool 90 according to the invention for cf-DNA detection and typing.

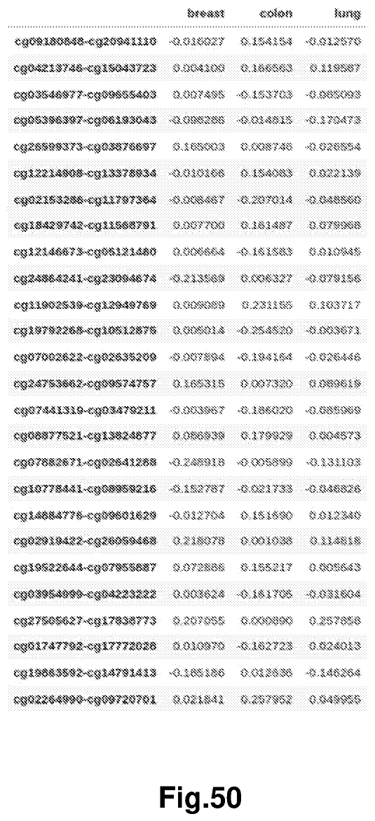

Custom reference atlas 80 [26 CpG pairs] for 3 tumour origin sites lung (n=1237), breast (n=791) and colon (n=457) from GDC repository [beta>=0.15, p-value=0.05] has been created. Such a reference atlas 80 for deconvolution containing reference profiles 60 for three different tissue types can be seen in FIG. 50. To estimate proportion of cf-DNA in a sample, a scaled, non-negative, LASSO regularized approximation was used. For testing such deconvolution tool 90 and the reference library 80 according to the invention, methylation profiles (EPIC) for 11 cf-DNA samples extracted from plasma from patients suffering from cancers placed in lung OR breast OR colon (Moss J, Magenheim J, Neiman D, Zemmour H, Loyfer N, Korach A, Samet Y, Maoz M, Druid H, Arner P, Fu K Y, Kiss E, Spalding K L, Landesberg G, Zick A, Grinshpun A, Shapiro A M J, Grompe M, Wittenberg A D, Glaser B, Shemer R, Kaplan T, Dor Y. Comprehensive human cell-type methylation atlas reveals origins of circulating cell-free DNA in health and disease) were used. In original study (Moss J, Magenheim J, Neiman D, Zemmour H, Loyfer N, Korach A, Samet Y, Maoz M, Druid H, Arner P, Fu K Y, Kiss E, Spalding K L, Landesberg G, Zick A, Grinshpun A, Shapiro A M J, Grompe M, Wittenberg A D, Glaser B, Shemer R, Kaplan T, Dor Y. Comprehensive human cell-type methylation atlas reveals origins of circulating cell-free DNA in health and disease), based on cf-DNA methylation profile, authors correctly identified source of 8 among 11 samples using atlas for 25 tissue/cell types and 7890 CpGs.

For each sample a proportion of cf-DNA using custom reference atlas 80 was estimated. It has been shown that using tools and data objects according to the invention, in particular using only 26 CpG pairs as methylation signatures, for 10 among 11 samples the origin of cancer was correctly indicated.

### Example 6

The sixth example relates to the discussion on accuracy of characterization of samples with the use of CpG pairs-based profiles according to the invention in view of available measurements methods, in particular in view of the trade-off between their costs and precision. Currently the most common measurement method used for measuring methylation levels is NGS. This method is very costly, because the accuracy of the measurements depends on so called depth of the measurements, namely on the average number of reads covering specific DNA fragment. In principle methylation measurements for specific DNA fragment using NGS (next-generation sequencing) technique is based on sequencing a library of short bisulfite pretreated DNA fragments (named reads) of sequence of interest. In case of the most commonly used NGS platform (Illumina) average read length is 150 bp. Therefore, to sequence (cover) DNA fragment of length 1000 bp around 7 reads are necessary (7*150 bp>1000 bp). The higher the depth the better is the accuracy of the measurements. As a consequence, sufficiently high depth of the measurements requires high number of reads what makes the NGS technology expensive. For example, in accordance with the above at least 600 million reads of 150 bp DNA fragments (or 300M paired-end reads) provides 30× average depth of human genome recognized as a standard guaranteeing good measurement accuracy for genomic research. Similar or higher depth (due to reduced complexity of the DNA after bisulfite modification) needs to be accomplished to study methylation levels at CpG sites across genome.

In contradiction to the above, such high number of reads to perform methylation measurements for single CpG sites is not required for generating CpG pairs-based profiles according to the invention. Firstly, because the relative value of methylation levels in a pair of adjacent CpG sites is necessary to establish methylation signatures, thus by calculating the relation of methylations values between two single CpG sites the measurement is not influenced by batch-effect. Secondly, since it is assumed that the distance of adjacent CpGs which constitute pairs used in profiles according to the invention is less than 150 bp, the length of sequences to be measured at once can be only 150 bp or less. As a consequence, data from single NGS read of sequence containing DMP is enough to generate valuable CpG pairs-based profiles according to the invention. The methods according to the invention are still efficient if digital data of methylation measurements are received from cheaper and simplified version of NGS for a set of single reads for sequences lengths under 150 bp.

Additionally for the same reason, valuable data on methylation levels for single CpG sites can be obtained from less common and much cheaper measurement methods, for example: pyrosequencing, Sanger sequencing or microarrays.

### Example 7

Another example describes the use of the CpG pairs-based profiles 20 according to the invention to estimate the chronological age and aberrations from the chronological age. For said purpose based on said CpG pairs-based profiles 20 for a group of samples for which the age of the subject is known another type of methylation map can be generated, namely an age associated methylation map.

In the analysis of 132 independent healthy white blood cells' methylomes containing 96 766 CpG pairs (data described in section 0061), the authors of the invention analyzed association between delta methylation level (difference of methylation levels as measured) in each CpG pair constituting said profiles 20 and patients' age. The authors of the invention identified 57309 CpG pairs associated with age (p-value<0.05, t-Student test). Genomic coordinates of such selected CpG pairs can be saved as an exemplary age associated methylation map. Said age associated methylation map can comprise one or more selected CpG pairs correlated with the chronological age.

That delta methylation of CpG pair or a set of CpG pairs can be used as a predictor in regression model to estimate chronological age based on delta methylation level of CpG pair or CpG pairs.

It is known for the skilled in the art (see for example Jain P, Binder A M, Chen B, Parada H Jr, Gallo L C, Alcaraz J, Horvath S, Bhatti P, Whitsel E A, Jordahl K, Baccarelli A A, Hou L, Stewart J D, Li Y, Justice J N, LaCroix A Z. Analysis of Epigenetic Age Acceleration and Healthy Longevity Among Older US Women) that discrepancy of estimated chronological age from the real chronological age indicates abnormal aging and can be an indicator of the disease. It is also known for the skilled in the art that age estimated on the bases of epigenetic modifications levels (referred as epigenetic age) can also be used as a biomarker of the pathological process.

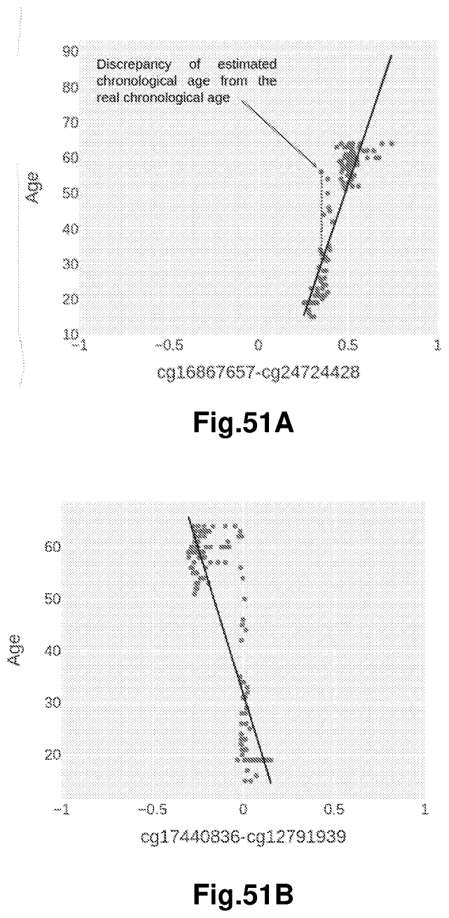

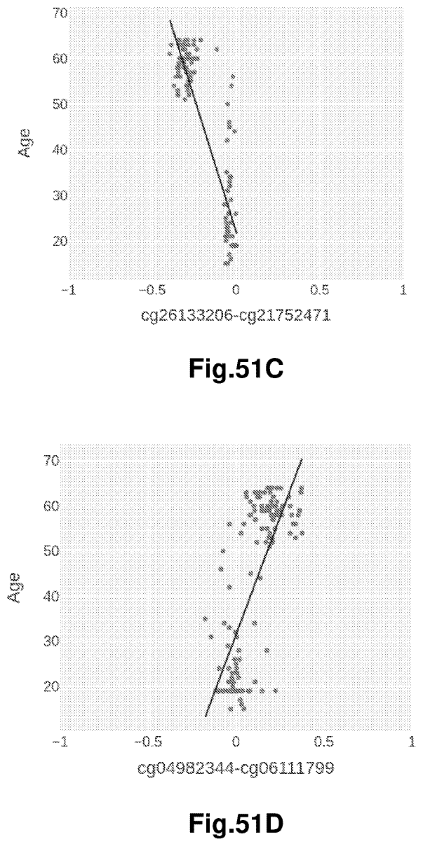

As it can be seen from 4 exemplary CpG pairs chronological age can be estimated (regression line) based on calculated difference of methylation levels for at least one specific CpG pair (see FIG. 51A-D). Exemplary discrepancy of estimated chronological age from the real chronological age is shown on FIG. 51A.

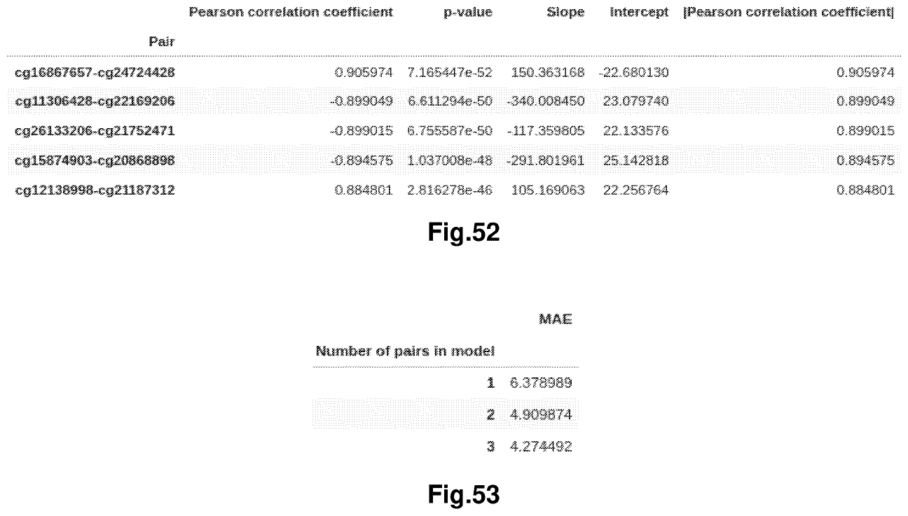

The authors of the invention further investigated how strongly the age is associated with CpG pairs-based age associated profiles 20′ according to the invention. Example results of statistical analysis for single exemplary CpG pairs are shown in FIG. 52. Expected improvement of assessment expressed as mean absolute error between predicted age and chronological age in function of CpG pairs number used for prediction is shown in FIG. 53.

In particular a chronological age model can be provided by training model using one of the regression models for example: linear regression, polynomial regression, spline regression, neural network or regression tree. These models provide a function that describes the association between delta methylation levels of one or more CpG pair(s) from a CpG pairs-based age associated methylation profile 20′ and sample chronological age. This function (model) can be used to predict biological age based on provided delta methylation levels contained in a CpG pairs-based age associated profile 20′, built with the use of the age associated methylation map.

The method for determining biological age of the subject can comprise the steps as follows. First, said computer implemented method for determining biological age of the subject, comprises providing an age associated methylation map; Then the second step involves providing a biological age regression model based on identified plurality of adjacent CpG pairs-based features indicative of chronological age; Next the method passes to a step of generating a CpG pairs-based age associated methylation profile, for a cell or tissue of the subject contained in a test sample, said CpG pairs-based age associated methylation profile containing genomics coordinates for adjacent CpG sites in each pair being correlated with chronological age and a single numerical value being the compressed methylation level information relating to said pair correlated with chronological age; Finally, the method passes to a step of determining the biological age of the subject by analyzing said CpG pairs-based age associated methylation profile for said test sample with the use of biological age regression model.

In one embodiment the step of providing an age associated methylation map comprises:
a) providing digital data on values of methylation level measurements for CpG sites contained in nucleic acid in a group of samples relating to said tissue or said cell, said group containing samples from subjects of different chronological age;
b) for each sample determining a set of pairs of adjacent CpG sites in said nucleic acid contained in said sample relating to said tissue or said cell, each pair of adjacent CpG sites consisting of two adjacent CpG sites localized within one nucleic acid molecule so as to generate a map of adjacent CpG sites containing genomic coordinates;
c) for each sample compressing information on methylation level value, iteratively for each pair of adjacent CpG sites among the determined set of pairs of adjacent CpG sites, by converting two values of methylation level associated with two CpG sites in each pair, into one single numerical value, said single value representing compressed methylation information for a pair of adjacent CpG sites;
d) selecting pairs of adjacent CpG sites for which said single value representing compressed methylation information is a CpG pairs-based feature indicative of chronological age, by checking, for said group of samples, for a group of single values representing compressed methylation information for specific CpG pair, an existence of correlation function with chronological age based on known chronological age of each sample;
e) saving genomic coordinates of adjacent CpG sites only for selected pairs which are correlated with chronological age so as to generate an age associated methylation map

In one embodiment the step of generating a CpG pairs-based age associated methylation profile comprises:
a) providing digital data on values of methylation level measurements for CpG sites contained in said nucleic acid in said sample relating to said tissue or said cell
b) determining a set of pairs of adjacent CpG sites in said nucleic acid contained in said sample relating to said tissue or said cell, each pair of adjacent CpG sites consisting of two adjacent CpG sites localized within one nucleic acid molecule so as to generate a map of adjacent CpG sites containing genomic coordinates;
c) compressing information on methylation level value, iteratively for each pair of adjacent CpG sites among the determined set of pairs of adjacent CpG sites, by converting two values of methylation level associated with two CpG sites in each pair, into one single numerical value, said single value representing compressed methylation information for a pair of adjacent CpG sites;
d) providing an age associated methylation map
e) saving genomic coordinates of adjacent CpG sites only for each pair which is correlated with chronological age and contained in the age associated methylation map and compressed methylation information in the form of one single numerical value for said each pair of adjacent CpG sites so as to generate an age profile based on pairs of adjacent CpG sites of said tissue or said cell.

In one embodiment the step of providing a biological age regression model comprises the following steps:
a) providing a training data set by
   providing a set of age associated methylation profiles based on pairs of adjacent CpG sites indicative of chronological age along with real chronological age for each age associated methylation profiles.
b) training the regression model with said training data set so as it is configured to predict biological age of a cell or tissue relating to an input cell or tissue contained in a test sample.
said tool being configured to output a result of the age prediction that indicates the biological age of the said test sample.

In another aspect the disclosure provides a computer implemented method for predicting health status of the subject, the method comprising
determining biological age by the method as described above,
then calculating difference between predicted biological age and real chronological age of the subject, the difference being indicative for general health status.

In another aspect the disclosure provides a tangible computer-readable medium comprising computer-readable code that, when executed by a computer, causes the computer to perform operations of the method for determining biological age of the subject comprising: a) identifying a plurality of adjacent CpG pairs-based features indicative of chronological age, wherein the plurality of adjacent CpG pairs-based features has a total number K of adjacent CpG pairs-based features, K being a positive integer, said features being the compressed methylation level information correlated with chronological age for a pair of adjacent CpG sites;
b) providing a biological age regression model based on identified plurality of adjacent CpG pairs-based features indicative of chronological age;
c) generating a CpG pairs-based age associated methylation profile, for a cell or tissue of the subject contained in a test sample, said CpG pairs-based methylation profile containing genomics coordinates for adjacent CpG sites in each pair being correlated with chronological age and a single numerical value being the compressed methylation level information relating to said pair correlated with chronological age;
d) determining the biological age of the subject by inputting said CpG pairs-based methylation profile into biological age regression model.

In another aspect the disclosure provides a tangible computer-readable medium comprising computer-readable code that, when executed by a computer, causes the computer to perform operations of the method for predicting health status of the subject comprising:
determining biological age by the method as described above,
calculating difference between predicted biological age and real chronological age of the subject, the difference being indicative for general health status.

1-70. (canceled)

71. A computer implemented method for constructing profile of a tissue or a cell, the profile being based on pairs of adjacent epigenetically modified bases, based on epigenetic modification information derived from nucleic acid containing epigenetically modified bases-contained in a sample relating to said tissue or a cell, the method comprising the following steps:
a) providing digital data on values of epigenetic modification level measurements for epigenetically modified bases contained in said nucleic acid in said sample relating to said tissue or said cell;
b) determining a set of pairs of adjacent epigenetically modified bases in said nucleic acid contained in said sample relating to said tissue or said cell, each pair of adjacent epigenetically modified bases consisting of two adjacent epigenetically modified bases localized within one nucleic acid molecule so as to generate a map of adjacent epigenetically modified bases containing genomic coordinates of each identified pair of adjacent epigenetically modified bases;
c) compressing information on epigenetic modification level value, iteratively for each pair of adjacent epigenetically modified bases among the determined set of pairs of adjacent epigenetically modified bases, by converting two values of epigenetic modification level associated with two epigenetically modified bases in each pair, into one single numerical value, said single value representing compressed epigenetic modification information for a pair of adjacent epigenetically modified bases and being indicative of epigenetic modification status in each pair of adjacent epigenetically modified bases;
d) saving
genomic coordinates of adjacent epigenetically modified bases for each pair in said determined set of pairs and
compressed epigenetic modification information in the form of one single numerical value for said each pair of adjacent epigenetically modified bases in said sample so as to generate a profile based on pairs of adjacent epigenetically modified bases of said tissue or said cell;
wherein each said pair of adjacent epigenetically modified bases is further localized at a distance less than a pre-defined threshold distance.

72. The method according to claim 71, wherein the epigenetic modification status is one among at least co-epigenetic modification status and non co-epigenetic modification status in a pair of adjacent epigenetically modified bases, preferably wherein epigenetic modification comprises one among methylation, hydroxymethylation, formylation or carboxylation, and the epigenetically modified base of the nucleic acid sequence is respectively a methylated base, a hydroxymethylated base, a formylated base, or a carboxylic acid containing base or a derivative thereof;
wherein the step c) preferably involves calculating epigenetic modification value difference between two epigenetically modified bases in each pair of said adjacent epigenetically modified bases;
wherein the step b) preferably comprises first a step of annotating said nucleic acid containing epigenetically modified base to a reference genome thereby determining genomic coordinates of each epigenetically modified base so as to generate a map of all epigenetically modified bases;
wherein step a) preferably comprises
either
providing said sample
extracting nucleic acid fragments from said sample
converting said extracted nucleic acid fragments
assessing epigenetic modification levels in said converted isolated nucleic acid so as to receive a plurality of values of epigenetic modification level measurements for said sample thereby providing digital data on values of epigenetic modification level measurements for epigenetically modified bases
or downloading data from publicly available databases.

73. The method according to claim 71, wherein the pre-defined threshold distance is less than 50 bp.

74. The method according to claim 72, wherein the pre-defined threshold distance is less than 50 bp.

75. A computer implemented method for constructing parametrized profile based on pairs of adjacent epigenetically modified bases of a known type of tissue or cell based on epigenetic modification information derived from epigenetically modified base-containing nucleic acid contained in a group of at least one sample, each sample relating to the same known type of said tissue or said cell, the method comprising:
a) for each sample in said group of at least one sample providing digital data on epigenetic modification level measurements for epigenetically modified bases contained in said nucleic acid in said sample relating to said tissue or said cell of a known type
b) determining a set of pairs of adjacent epigenetically modified bases in said nucleic acid contained in each sample in said group of at least one sample relating to said tissue or said cell of a known type, each pair of adjacent epigenetically modified bases consisting of two adjacent epigenetically modified bases localized within one nucleic acid molecule so as to generate a map of adjacent epigenetically modified bases containing genomic coordinates; then
c) for said group of at least one sample iteratively for each pair of adjacent epigenetically modified bases among the determined set of pairs of adjacent epigenetically modified bases, compressing information on epigenetic modification level value, by fitting a parametrized mathematical model to said digital data on values of epigenetic modification level measurements associated with each pair of adjacent epigenetically modified bases so as to determine one single model parameter, said single model parameter representing compressed epigenetic modification information for a pair of adjacent epigenetically modified bases and being indicative of epigenetic modification status in each pair of adjacent epigenetically modified bases for said group of at least one sample;
d) for said group of at least one sample saving
genomic coordinates of adjacent epigenetically modified bases for each pair in said determined set of pairs and
compressed epigenetic modification information in the form of one model parameter for said each pair of adjacent epigenetically modified bases in said group of at least one sample
so as to generate said parametrized profile based on pairs of adjacent epigenetically modified bases of a known type of tissue or cell;
preferably wherein the epigenetic modification status is one among at least co-epigenetic modification status and non co-epigenetic modification status in a pair of adjacent epigenetically modified bases;

76. The method according to claim 75, wherein the step c) comprises: for each pair of adjacent epigenetically modified bases separately, estimating a regression model such that:
$$MetLevel ∼ intercept + {β}_{i} * POS$$
$$POS = \{ \begin{matrix}0 & if CpG = first \\ 1 & if CpG = successive\end{matrix}$$
wherein intercept and βᵢ, are model parameters, the parameter POS being an exogenous variable taking values of 0 if the epigenetically modified base in a pair is a first one, or 1 if the epigenetically modified base in a pair is a successive one; preferably
wherein if βᵢ≠0 the non co-epigenetic modification status is further one among negative sign non co-epigenetic modification status for βᵢ<0 and positive sign non co-epigenetic modification status for βᵢ>0;
preferably wherein the statistical significance assessment of the value of the βᵢ parameter is performed using t-Student test to calculate and save the value of empirical probability (p-value).

77. The method according to claim 75, wherein the step c) comprises
a) determining, separately for each first and each successive epigenetically modified base in each same pair of adjacent epigenetically modified bases across said group of at least one sample, a confidence interval for the mean epigenetic modification level such that:
$$P ( \overline{X} - {t}_{\text{?}} \frac{S}{\sqrt{n}} < μ < \overline{X} + {t}_{\text{?}} \frac{S}{\sqrt{n}} ) = 1 - \text{?} ,$$
$$\text{?} \text{indicates text missing or illegible when filed}$$
wherein alfa is a predefined parameter, advantageously equal to 0.05, n—the number of samples in the set of samples, S—standard deviation from the epigenetic modification level in said group of samples for each epigenetically modified base in the pair, 1 1-alfa 12 the quantile of order 1-alfa/2 of student's t-distribution with n−1 degrees of freedom.
b) determining a model parameter being the distance pi between the confidence interval calculated for the first epigenetically modified base and the confidence interval calculated for the successive; preferably
wherein the selected measure of distance βᵢ is:
the Chebyshev measure
$${D}_{Chebyshev} ( x , y ) := {max}_{i} ( \vert {x}_{i} - {y}_{i} \vert )$$

78. The method according to claim 75, wherein the step c) comprises
a) determining, separately for each first and each successive epigenetically modified base in each same pair of adjacent epigenetically modified bases across said group of samples, mean or median level of epigenetic modification
b) determining a model parameter βᵢ being the absolute value of difference between mean levels of epigenetic modification of the epigenetically modified bases for the first epigenetically modified base and the successive epigenetically modified base; preferably
wherein it further comprises testing significance of said calculated difference between mean levels of epigenetic modification of the epigenetically modified bases using statistical tests such as t-test, ANOVA, Mann-Whitney U or Kruskal-Wallis to calculate and save the value of empirical probability (p-value) for each pair of adjacent epigenetically modified bases.

79. A computer implemented method for constructing a discriminative map of cells or tissues types based on pairs of adjacent epigenetically modified bases of a known cell type or tissue type within a specific application context, the method comprising:
a) providing at least two parametrized profiles based on pairs of adjacent epigenetically modified bases for at least two different known types of cells or tissues generated with the method according to claim 75, wherein the number and the known types of cells or tissues, for which the parametrized profiles are provided, giving a specific application context.
b) determining and comparing the epigenetic modification status contained in the model parameters for each pair of adjacent epigenetically modified bases present in all of said at least two parametrized profiles
c) extracting each pair to be a part of said discriminative map of cells or tissues types under construction if the epigenetic modification status in at least one parametrized profile for said pair is different from the epigenetic modification status in at least one among all other parametrized profiles.
d) saving genomic coordinates for extracted pairs of adjacent epigenetically modified bases so as to generate said discriminative map of cells or tissues types for a specific application context;
preferably in the step b) it is determined that
if βᵢ is approximately equal to 0, then the epigenetic modification level of both epigenetically modified bases in the pair is the same and the epigenetic modification status for said pair is co-epigenetic modification status or that
if the absolute value of the parameter βᵢ>0, then the epigenetic modification level of both epigenetically modified bases in the pair is different and the epigenetic modification status for said pair is non co-epigenetic modification status; more preferably in the step b) if the parameter p-value is available and the p-value is higher than a statistical significance threshold then it is determined that Pi is equal to 0 then the epigenetic modification level of both epigenetically modified bases in the pair is the same and the epigenetic modification status for said pair is co-epigenetic modification status or in the step b) the non co-epigenetic modification status is determined for I βᵢ I>β threshold value, wherein the β threshold value being a value in the range from 0 to 1 excluding 0 and 1;
preferably in the step c) a pair of adjacent epigenetically modified bases is extracted to be a part of said discriminative map of cells or tissues types under construction if the epigenetic modification status in one parametrized profile for said pair is a non comethylation status and the epigenetic modification status in any other parametrized profiles is a comethylation status.

80. A computer implemented method for constructing a discriminative map of cells or tissues types based on pairs of adjacent epigenetically modified bases of a known cell type or tissue type within a specific application context, the method comprising:
a) providing at least two parametrized profiles based on pairs of adjacent epigenetically modified bases for at least two different known types of cells or tissues generated with the method according to claim 76, wherein the number and the known types of cells or tissues, for which the parametrized profiles are provided, giving a specific application context.
b) determining and comparing the epigenetic modification status contained in the model parameters for each pair of adjacent epigenetically modified bases present in all of said at least two parametrized profiles
c) extracting each pair to be a part of said discriminative map of cells or tissues types under construction if the epigenetic modification status in at least one parametrized profile for said pair is different from the epigenetic modification status in at least one among all other parametrized profiles.
d) saving genomic coordinates for extracted pairs of adjacent epigenetically modified bases so as to generate said discriminative map of cells or tissues types for a specific application context;
preferably in the step b) it is determined that
if βᵢ is approximately equal to 0, then the epigenetic modification level of both epigenetically modified bases in the pair is the same and the epigenetic modification status for said pair is co-epigenetic modification status or that
if the absolute value of the parameter βᵢ>0, then the epigenetic modification level of both epigenetically modified bases in the pair is different and the epigenetic modification status for said pair is non co-epigenetic modification status; more preferably in the step b) if the parameter p-value is available and the p-value is higher than a statistical significance threshold then it is determined that βᵢ is equal to 0 then the epigenetic modification level of both epigenetically modified bases in the pair is the same and the epigenetic modification status for said pair is co-epigenetic modification status or in the step b) the non co-epigenetic modification status is determined for I βᵢ I>β threshold value, wherein the β threshold value being a value in the range from 0 to 1 excluding 0 and 1;
preferably in the step c) a pair of adjacent epigenetically modified bases is extracted to be a part of said discriminative map of cells or tissues types under construction if the epigenetic modification status in one parametrized profile for said pair is a non comethylation status and the epigenetic modification status in any other parametrized profiles is a comethylation status.

81. A computer implemented method for constructing a discriminative map of cells or tissues types based on pairs of adjacent epigenetically modified bases of a known cell type or tissue type within a specific application context, the method comprising:
a) providing at least two parametrized profiles based on pairs of adjacent epigenetically modified bases for at least two different known types of cells or tissues generated with the method according to any of to claim 77, wherein the number and the known types of cells or tissues, for which the parametrized profiles are provided, giving a specific application context.
b) determining and comparing the epigenetic modification status contained in the model parameters for each pair of adjacent epigenetically modified bases present in all of said at least two parametrized profiles
c) extracting each pair to be a part of said discriminative map of cells or tissues types under construction if the epigenetic modification status in at least one parametrized profile for said pair is different from the epigenetic modification status in at least one among all other parametrized profiles.
d) saving genomic coordinates for extracted pairs of adjacent epigenetically modified bases so as to generate said discriminative map of cells or tissues types for a specific application context;
preferably in the step b) it is determined that
if βᵢ is approximately equal to 0, then the epigenetic modification level of both epigenetically modified bases in the pair is the same and the epigenetic modification status for said pair is co-epigenetic modification status or that
if the absolute value of the parameter βᵢ>0, then the epigenetic modification level of both epigenetically modified bases in the pair is different and the epigenetic modification status for said pair is non co-epigenetic modification status; more preferably in the step b) if the parameter p-value is available and the p-value is higher than a statistical significance threshold then it is determined that βᵢ is equal to 0 then the epigenetic modification level of both epigenetically modified bases in the pair is the same and the epigenetic modification status for said pair is co-epigenetic modification status or in the step b) the non co-epigenetic modification status is determined for I βᵢ I>β threshold value, wherein the β threshold value being a value in the range from 0 to 1 excluding 0 and 1;
preferably in the step c) a pair of adjacent epigenetically modified bases is extracted to be a part of said discriminative map of cells or tissues types under construction if the epigenetic modification status in one parametrized profile for said pair is a non comethylation status and the epigenetic modification status in any other parametrized profiles is a comethylation status.

82. A computer implemented method for constructing a discriminative map of cells or tissues types based on pairs of adjacent epigenetically modified bases of a known cell type or tissue type within a specific application context, the method comprising:
a) providing at least two parametrized profiles based on pairs of adjacent epigenetically modified bases for at least two different known types of cells or tissues generated with the method according to claim 78, wherein the number and the known types of cells or tissues, for which the parametrized profiles are provided, giving a specific application context.
b) determining and comparing the epigenetic modification status contained in the model parameters for each pair of adjacent epigenetically modified bases present in all of said at least two parametrized profiles
c) extracting each pair to be a part of said discriminative map of cells or tissues types under construction if the epigenetic modification status in at least one parametrized profile for said pair is different from the epigenetic modification status in at least one among all other parametrized profiles.
d) saving genomic coordinates for extracted pairs of adjacent epigenetically modified bases so as to generate said discriminative map of cells or tissues types for a specific application context;
preferably in the step b) it is determined that
if βᵢ is approximately equal to 0, then the epigenetic modification level of both epigenetically modified bases in the pair is the same and the epigenetic modification status for said pair is co-epigenetic modification status or that
if the absolute value of the parameter βᵢ>0, then the epigenetic modification level of both epigenetically modified bases in the pair is different and the epigenetic modification status for said pair is non co-epigenetic modification status; more preferably in the step b) if the parameter p-value is available and the p-value is higher than a statistical significance threshold then it is determined that βᵢ is equal to 0 then the epigenetic modification level of both epigenetically modified bases in the pair is the same and the epigenetic modification status for said pair is co-epigenetic modification status or in the step b) the non co-epigenetic modification status is determined for I βᵢ I>β threshold value, wherein the β threshold value being a value in the range from 0 to 1 excluding 0 and 1;
preferably in the step c) a pair of adjacent epigenetically modified bases is extracted to be a part of said discriminative map of cells or tissues types under construction if the epigenetic modification status in one parametrized profile for said pair is a non comethylation status and the epigenetic modification status in any other parametrized profiles is a comethylation status.

83. A computer implemented method for constructing cell or tissue type discriminative profile based on pairs of adjacent epigenetically modified bases of nucleic acid contained in a sample relating to a certain type of tissue or cell within a specific application context, the method comprising:
a) for said sample relating to a certain type of tissue or cell providing its profile based on pairs of adjacent epigenetically modified bases, said profile being generated with the use of the method according to claim 71,
b) providing a discriminative map of cells or tissues types based on pairs of adjacent epigenetically modified bases, said map being generated for said specific application context with the use of the method according to claim 79,
c) saving, only for pairs of adjacent epigenetically modified bases contained in said discriminative map of cell or tissues types,
genomic coordinates of adjacent epigenetically modified bases and
compressed epigenetic modification information in the form of one single numerical value relating to said adjacent epigenetically modified bases,
so as to generate said cell or tissue type discriminative profile based on pairs of adjacent epigenetically modified bases for said sample relating to a certain type of tissue or cell within said specific application context;
preferably method further comprises either:
providing a model for cell or tissue type classification comprising:
a) providing a training data set by
providing a first set of cell or tissue type discriminative profiles based on pairs of adjacent epigenetically modified bases within a specific application context for the first known type of the tissue or cell along with associated labels indicating the first known type of tissue or cell
providing at least a second set of cell or tissue type discriminative profile based on pairs of adjacent epigenetically modified bases within a specific application context for at least the second known type of the tissue or cell along with associated labels indicating the at least second known type of tissue or cell, the first and at least the second set of cell or tissue type discriminative profiles;
b) training the classification model with said training data set so as it is configured to classify the type of a cell or tissue relating to an input cell or tissue type discriminative profile;
and/or
classifying a tissue or cell of origin contained in a test sample by
a) inputting said discriminative profile based on pairs of adjacent epigenetically modified bases for said test sample into a trained classification model;
b) receiving a result of the classification that indicates the tissue or cell of origin contained in said test sample;
and/or
constructing reference discriminative profile of a known cell or tissue type based on pairs of adjacent epigenetically modified bases within a specific application context by
a) providing a set of discriminative profiles based on pairs of adjacent epigenetically modified bases within said specific application context for a group of at least one sample of the same known type of the tissue or cell;
b) for each pair of adjacent epigenetically modified bases in said discriminative profiles, for all samples in said group of at least one sample of the same cell or tissue type, calculating one among a mean or median value resulting from a set of compressed epigenetic modification information in the form of one single numerical value relating to said each pair for said all samples in said group of at least one samples;
c) saving
genomic coordinates of each pair of adjacent epigenetically modified bases and
said calculated mean value or median of the compressed epigenetic modification information in the form of one single numerical value for said each pair
so as to generate a reference discriminative profile of a known cell or tissue type based on pairs of adjacent epigenetically modified bases within a specific application context;
preferably further comprising a method for generating a library of reference discriminative profiles of cell or tissue types based on pairs of adjacent epigenetically modified bases of known types of tissue or cell for specific application, the method comprising:
a) providing a first reference discriminative profile of cell or tissue types based on pairs of adjacent epigenetically modified bases within a specific application context for a first known type of cell or tissue;
b) providing at least a second reference discriminative profile of cell or tissue types based on pairs of adjacent epigenetically modified bases within a specific application context for at least a second known type of cell or tissue, said first and at least second reference discriminative profiles of cell or tissue types;
c) storing said at least two reference discriminative profiles of cell or tissue types based on pairs of adjacent epigenetically modified bases so as to generate said library of reference discriminative profiles;
preferably further comprising a method for determining a tissue or cell of origin contained in a test sample, the method comprising:
a) providing a library of reference discriminative profiles of cell or tissue types based on pairs of adjacent epigenetically modified bases,
b) providing a querying tool;
c) providing for said test sample a discriminative profile based on pairs of adjacent epigenetically modified bases, said received discriminative profile
d) querying said library of reference discriminative profiles with the use of the querying tool and said discriminative profile of said test sample; and
e) receiving a result of the query that indicates the tissue or cell of origin contained in said test sample;
preferably further comprising a method for constructing reduced cell or tissue type discriminative profile based on pairs of adjacent epigenetically modified bases of known types of tissue or cell within a specific application context:
a) providing the classification model;
b) providing a training data set being a first and at least a second set of cell or tissue type discriminative profiles based on pairs of adjacent epigenetically modified bases for at least two different types of cell or tissue along with associated labels indicating the known type of tissue or cell;
c) eliminating unnecessary pairs of adjacent epigenetically modified bases in the training data set by further training said classification model with the use of a feature selection method;
d) saving reduced discriminative map;
e) saving said reduced trained model;
preferably further comprising a method for predicting a sample composition of a test sample, the method comprising:
a) providing a library of reference discriminative profiles of cell or tissue types based on pairs of adjacent epigenetically modified bases, the library being obtained by the method according to claim 28;
b) providing a deconvolution tool;
c) providing for said test sample a discriminative profile based on pairs of adjacent epigenetically modified bases, said received discriminative profile
d) performing deconvolution with the use of the deconvolution tool based on data contained in the library of reference discriminative profiles and contained in said discriminative profile of the test sample; and
e) receiving a result of the deconvolution that indicates the composition of said test sample.

84. A computer implemented method for constructing cell or tissue type discriminative profile based on pairs of adjacent epigenetically modified bases of nucleic acid contained in a sample relating to a certain type of tissue or cell within a specific application context, the method comprising:
a) for said sample relating to a certain type of tissue or cell providing its profile based on pairs of adjacent epigenetically modified bases, said profile being generated with the use of the method according to claim 72,
b) providing a discriminative map of cells or tissues types based on pairs of adjacent epigenetically modified bases, said map being generated for said specific application context with the use of the method according to claim 79,
c) saving, only for pairs of adjacent epigenetically modified bases contained in said discriminative map of cell or tissues types,
genomic coordinates of adjacent epigenetically modified bases and
compressed epigenetic modification information in the form of one single numerical value relating to said adjacent epigenetically modified bases,
so as to generate said cell or tissue type discriminative profile based on pairs of adjacent epigenetically modified bases for said sample relating to a certain type of tissue or cell within said specific application context;
preferably method further comprises either:
providing a model for cell or tissue type classification comprising:
a) providing a training data set by
providing a first set of cell or tissue type discriminative profiles based on pairs of adjacent epigenetically modified bases within a specific application context for the first known type of the tissue or cell along with associated labels indicating the first known type of tissue or cell
providing at least a second set of cell or tissue type discriminative profile based on pairs of adjacent epigenetically modified bases within a specific application context for at least the second known type of the tissue or cell along with associated labels indicating the at least second known type of tissue or cell, the first and at least the second set of cell or tissue type discriminative profiles;
b) training the classification model with said training data set so as it is configured to classify the type of a cell or tissue relating to an input cell or tissue type discriminative profile;
and/or
classifying a tissue or cell of origin contained in a test sample by
a) inputting said discriminative profile based on pairs of adjacent epigenetically modified bases for said test sample into a trained classification model;
b) receiving a result of the classification that indicates the tissue or cell of origin contained in said test sample;
and/or
constructing reference discriminative profile of a known cell or tissue type based on pairs of adjacent epigenetically modified bases within a specific application context by
a) providing a set of discriminative profiles based on pairs of adjacent epigenetically modified bases within said specific application context for a group of at least one sample of the same known type of the tissue or cell;
b) for each pair of adjacent epigenetically modified bases in said discriminative profiles, for all samples in said group of at least one sample of the same cell or tissue type, calculating one among a mean or median value resulting from a set of compressed epigenetic modification information in the form of one single numerical value relating to said each pair for said all samples in said group of at least one samples;
c) saving
genomic coordinates of each pair of adjacent epigenetically modified bases and
said calculated mean value or median of the compressed epigenetic modification information in the form of one single numerical value for said each pair
so as to generate a reference discriminative profile of a known cell or tissue type based on pairs of adjacent epigenetically modified bases within a specific application context;
preferably further comprising a method for generating a library of reference discriminative profiles of cell or tissue types based on pairs of adjacent epigenetically modified bases of known types of tissue or cell for specific application, the method comprising:
a) providing a first reference discriminative profile of cell or tissue types based on pairs of adjacent epigenetically modified bases within a specific application context for a first known type of cell or tissue;
b) providing at least a second reference discriminative profile of cell or tissue types based on pairs of adjacent epigenetically modified bases within a specific application context for at least a second known type of cell or tissue, said first and at least second reference discriminative profiles of cell or tissue types;
c) storing said at least two reference discriminative profiles of cell or tissue types based on pairs of adjacent epigenetically modified bases so as to generate said library of reference discriminative profiles;
preferably further comprising a method for determining a tissue or cell of origin contained in a test sample, the method comprising:
a) providing a library of reference discriminative profiles of cell or tissue types based on pairs of adjacent epigenetically modified bases,
b) providing a querying tool;
c) providing for said test sample a discriminative profile based on pairs of adjacent epigenetically modified bases, said received discriminative profile
d) querying said library of reference discriminative profiles with the use of the querying tool and said discriminative profile of said test sample; and
e) receiving a result of the query that indicates the tissue or cell of origin contained in said test sample;
preferably further comprising a method for constructing reduced cell or tissue type discriminative profile based on pairs of adjacent epigenetically modified bases of known types of tissue or cell within a specific application context:
a) providing the classification model;
b) providing a training data set being a first and at least a second set of cell or tissue type discriminative profiles based on pairs of adjacent epigenetically modified bases for at least two different types of cell or tissue along with associated labels indicating the known type of tissue or cell;
c) eliminating unnecessary pairs of adjacent epigenetically modified bases in the training data set by further training said classification model with the use of a feature selection method;
d) saving reduced discriminative map;
e) saving said reduced trained model;
preferably further comprising a method for predicting a sample composition of a test sample, the method comprising:
a) providing a library of reference discriminative profiles of cell or tissue types based on pairs of adjacent epigenetically modified bases, the library being obtained by the method according to claim 28;
b) providing a deconvolution tool;
c) providing for said test sample a discriminative profile based on pairs of adjacent epigenetically modified bases, said received discriminative profile
d) performing deconvolution with the use of the deconvolution tool based on data contained in the library of reference discriminative profiles and contained in said discriminative profile of the test sample; and
e) receiving a result of the deconvolution that indicates the composition of said test sample.

85. A computer implemented method for constructing cell or tissue type discriminative profile based on pairs of adjacent epigenetically modified bases of nucleic acid contained in a sample relating to a certain type of tissue or cell within a specific application context, the method comprising:
a) for said sample relating to a certain type of tissue or cell providing its profile based on pairs of adjacent epigenetically modified bases, said profile being generated with the use of the method according to claim 73,
b) providing a discriminative map of cells or tissues types based on pairs of adjacent epigenetically modified bases, said map being generated for said specific application context with the use of the method according to claim 79,
c) saving, only for pairs of adjacent epigenetically modified bases contained in said discriminative map of cell or tissues types,
genomic coordinates of adjacent epigenetically modified bases and
compressed epigenetic modification information in the form of one single numerical value relating to said adjacent epigenetically modified bases,
so as to generate said cell or tissue type discriminative profile based on pairs of adjacent epigenetically modified bases for said sample relating to a certain type of tissue or cell within said specific application context;
preferably method further comprises either:
providing a model for cell or tissue type classification comprising:
a) providing a training data set by
providing a first set of cell or tissue type discriminative profiles based on pairs of adjacent epigenetically modified bases within a specific application context for the first known type of the tissue or cell along with associated labels indicating the first known type of tissue or cell
providing at least a second set of cell or tissue type discriminative profile based on pairs of adjacent epigenetically modified bases within a specific application context for at least the second known type of the tissue or cell along with associated labels indicating the at least second known type of tissue or cell, the first and at least the second set of cell or tissue type discriminative profiles;
b) training the classification model with said training data set so as it is configured to classify the type of a cell or tissue relating to an input cell or tissue type discriminative profile;
and/or
classifying a tissue or cell of origin contained in a test sample by
a) inputting said discriminative profile based on pairs of adjacent epigenetically modified bases for said test sample into a trained classification model;
b) receiving a result of the classification that indicates the tissue or cell of origin contained in said test sample;
and/or
constructing reference discriminative profile of a known cell or tissue type based on pairs of adjacent epigenetically modified bases within a specific application context by
a) providing a set of discriminative profiles based on pairs of adjacent epigenetically modified bases within said specific application context for a group of at least one sample of the same known type of the tissue or cell;
b) for each pair of adjacent epigenetically modified bases in said discriminative profiles, for all samples in said group of at least one sample of the same cell or tissue type, calculating one among a mean or median value resulting from a set of compressed epigenetic modification information in the form of one single numerical value relating to said each pair for said all samples in said group of at least one samples;
c) saving
genomic coordinates of each pair of adjacent epigenetically modified bases and
said calculated mean value or median of the compressed epigenetic modification information in the form of one single numerical value for said each pair
so as to generate a reference discriminative profile of a known cell or tissue type based on pairs of adjacent epigenetically modified bases within a specific application context;
preferably further comprising a method for generating a library of reference discriminative profiles of cell or tissue types based on pairs of adjacent epigenetically modified bases of known types of tissue or cell for specific application, the method comprising:
a) providing a first reference discriminative profile of cell or tissue types based on pairs of adjacent epigenetically modified bases within a specific application context for a first known type of cell or tissue;
b) providing at least a second reference discriminative profile of cell or tissue types based on pairs of adjacent epigenetically modified bases within a specific application context for at least a second known type of cell or tissue, said first and at least second reference discriminative profiles of cell or tissue types;
c) storing said at least two reference discriminative profiles of cell or tissue types based on pairs of adjacent epigenetically modified bases so as to generate said library of reference discriminative profiles;
preferably further comprising a method for determining a tissue or cell of origin contained in a test sample, the method comprising:
a) providing a library of reference discriminative profiles of cell or tissue types based on pairs of adjacent epigenetically modified bases,
b) providing a querying tool;
c) providing for said test sample a discriminative profile based on pairs of adjacent epigenetically modified bases, said received discriminative profile
d) querying said library of reference discriminative profiles with the use of the querying tool and said discriminative profile of said test sample; and
e) receiving a result of the query that indicates the tissue or cell of origin contained in said test sample;
preferably further comprising a method for constructing reduced cell or tissue type discriminative profile based on pairs of adjacent epigenetically modified bases of known types of tissue or cell within a specific application context:
a) providing the classification model;
b) providing a training data set being a first and at least a second set of cell or tissue type discriminative profiles based on pairs of adjacent epigenetically modified bases for at least two different types of cell or tissue along with associated labels indicating the known type of tissue or cell;
c) eliminating unnecessary pairs of adjacent epigenetically modified bases in the training data set by further training said classification model with the use of a feature selection method;
d) saving reduced discriminative map;
e) saving said reduced trained model;
preferably further comprising a method for predicting a sample composition of a test sample, the method comprising:
a) providing a library of reference discriminative profiles of cell or tissue types based on pairs of adjacent epigenetically modified bases, the library being obtained by the method according to claim 28;
b) providing a deconvolution tool;
c) providing for said test sample a discriminative profile based on pairs of adjacent epigenetically modified bases, said received discriminative profile
d) performing deconvolution with the use of the deconvolution tool based on data contained in the library of reference discriminative profiles and contained in said discriminative profile of the test sample; and
e) receiving a result of the deconvolution that indicates the composition of said test sample.

86. A computer implemented method for constructing cell or tissue type discriminative profile based on pairs of adjacent epigenetically modified bases of nucleic acid contained in a sample relating to a certain type of tissue or cell within a specific application context, the method comprising:
a) for said sample relating to a certain type of tissue or cell providing its profile based on pairs of adjacent epigenetically modified bases, said profile being generated with the use of the method according to claim 74,
b) providing a discriminative map of cells or tissues types based on pairs of adjacent epigenetically modified bases, said map being generated for said specific application context with the use of the method according to claim 79,
c) saving, only for pairs of adjacent epigenetically modified bases contained in said discriminative map of cell or tissues types,
genomic coordinates of adjacent epigenetically modified bases and
compressed epigenetic modification information in the form of one single numerical value relating to said adjacent epigenetically modified bases,
so as to generate said cell or tissue type discriminative profile based on pairs of adjacent epigenetically modified bases for said sample relating to a certain type of tissue or cell within said specific application context;
preferably method further comprises either:
providing a model for cell or tissue type classification comprising:
a) providing a training data set by
providing a first set of cell or tissue type discriminative profiles based on pairs of adjacent epigenetically modified bases within a specific application context for the first known type of the tissue or cell along with associated labels indicating the first known type of tissue or cell
providing at least a second set of cell or tissue type discriminative profile based on pairs of adjacent epigenetically modified bases within a specific application context for at least the second known type of the tissue or cell along with associated labels indicating the at least second known type of tissue or cell, the first and at least the second set of cell or tissue type discriminative profiles;
b) training the classification model with said training data set so as it is configured to classify the type of a cell or tissue relating to an input cell or tissue type discriminative profile;
and/or
classifying a tissue or cell of origin contained in a test sample by
a) inputting said discriminative profile based on pairs of adjacent epigenetically modified bases for said test sample into a trained classification model;
b) receiving a result of the classification that indicates the tissue or cell of origin contained in said test sample;
and/or
constructing reference discriminative profile of a known cell or tissue type based on pairs of adjacent epigenetically modified bases within a specific application context by
a) providing a set of discriminative profiles based on pairs of adjacent epigenetically modified bases within said specific application context for a group of at least one sample of the same known type of the tissue or cell;
b) for each pair of adjacent epigenetically modified bases in said discriminative profiles, for all samples in said group of at least one sample of the same cell or tissue type, calculating one among a mean or median value resulting from a set of compressed epigenetic modification information in the form of one single numerical value relating to said each pair for said all samples in said group of at least one samples;
c) saving
genomic coordinates of each pair of adjacent epigenetically modified bases and
said calculated mean value or median of the compressed epigenetic modification information in the form of one single numerical value for said each pair
so as to generate a reference discriminative profile of a known cell or tissue type based on pairs of adjacent epigenetically modified bases within a specific application context;
preferably further comprising a method for generating a library of reference discriminative profiles of cell or tissue types based on pairs of adjacent epigenetically modified bases of known types of tissue or cell for specific application, the method comprising:
a) providing a first reference discriminative profile of cell or tissue types based on pairs of adjacent epigenetically modified bases within a specific application context for a first known type of cell or tissue;
b) providing at least a second reference discriminative profile of cell or tissue types based on pairs of adjacent epigenetically modified bases within a specific application context for at least a second known type of cell or tissue, said first and at least second reference discriminative profiles of cell or tissue types;
c) storing said at least two reference discriminative profiles of cell or tissue types based on pairs of adjacent epigenetically modified bases so as to generate said library of reference discriminative profiles;
preferably further comprising a method for determining a tissue or cell of origin contained in a test sample, the method comprising:
a) providing a library of reference discriminative profiles of cell or tissue types based on pairs of adjacent epigenetically modified bases,
b) providing a querying tool;
c) providing for said test sample a discriminative profile based on pairs of adjacent epigenetically modified bases, said received discriminative profile
d) querying said library of reference discriminative profiles with the use of the querying tool and said discriminative profile of said test sample; and
e) receiving a result of the query that indicates the tissue or cell of origin contained in said test sample;
preferably further comprising a method for constructing reduced cell or tissue type discriminative profile based on pairs of adjacent epigenetically modified bases of known types of tissue or cell within a specific application context:
a) providing the classification model;
b) providing a training data set being a first and at least a second set of cell or tissue type discriminative profiles based on pairs of adjacent epigenetically modified bases for at least two different types of cell or tissue along with associated labels indicating the known type of tissue or cell;
c) eliminating unnecessary pairs of adjacent epigenetically modified bases in the training data set by further training said classification model with the use of a feature selection method;
d) saving reduced discriminative map;
e) saving said reduced trained model;
preferably further comprising a method for predicting a sample composition of a test sample, the method comprising:
a) providing a library of reference discriminative profiles of cell or tissue types based on pairs of adjacent epigenetically modified bases, the library being obtained by the method according to claim 28;
b) providing a deconvolution tool;
c) providing for said test sample a discriminative profile based on pairs of adjacent epigenetically modified bases, said received discriminative profile
d) performing deconvolution with the use of the deconvolution tool based on data contained in the library of reference discriminative profiles and contained in said discriminative profile of the test sample; and
e) receiving a result of the deconvolution that indicates the composition of said test sample.

87. A computer implemented method of characterizing a test sample from a subject, comprising:
a) receiving methylation level data provided by measuring methylation level of consecutive nucleic acid sequence contained in said test sample from the subject;
b) providing for said test sample a CpG pair-based discriminative methylation profile based on methylation level data, wherein the CpG pair-based methylation discriminative profile comprises a differential methylation pairs (DMPs) selected based on specificity of methylation status in said DMPs in at least one sample type among at least one from another sample types, said profile containing genomic coordinates of said consecutive DMPs and a single value indicative of methylation status for said DMPs in said sample;
c) inputting said CpG pair-based methylation profile to a sample characterizing tool, in order to determine sample type, said tool outputting the result of sample characterization and using for characterization one or more pre-established methylation signatures, wherein each of the one or more pre-established methylation signatures correlates with sample type, and wherein each pre-established methylation signature comprises a set of differential methylation pairs DMP selected based on specificity of methylation status in said DMPs in at least one sample type among at least one from another sample types;
preferably, in the step a) measuring methylation level of consecutive nucleic acid sequence contained in said test sample from the subject comprises
providing said test sample
extracting nucleic acid fragments from said sample
converting said extracted nucleic acid fragments
assessing methylation levels in said converted isolated nucleic acid so as to receive a plurality of values of methylation level measurements for said sample thereby providing digital data on values of methylation level measurements for CpG sites;
preferably, the step b) comprises
A1) for said test sample relating to a certain type of tissue or cell providing its profile based on pairs of adjacent CpG sites, preferably by:
i) determining a set of pairs of adjacent CpG sites in said nucleic acid contained in said test sample, each pair of adjacent CpG sites consisting of two adjacent CpG sites localized within one nucleic acid molecule so as to generate a map of adjacent CpG sites containing genomic coordinates;
ii) compressing information on methylation level value, iteratively for each pair of adjacent CpG sites among the determined set of pairs of adjacent CpG sites, by converting two values of methylation level associated with two CpG sites in each pair, into one single numerical value, said single value representing compressed methylation information for a pair of adjacent CpG sites and being indicative of methylation status in each pair of adjacent CpG sites;
iii) saving
genomic coordinates of adjacent CpG sites for each pair in said determined set of pairs and
compressed methylation information in the form of one single numerical value for said each pair of adjacent CpG sites in said sample so as to generate a profile based on pairs of adjacent CpG sites of said tissue or said cell;
B1) providing a discriminative map of cells or tissues types based on pairs of adjacent CpG sites, said map being generated for said specific application context and containing only differential methylation pairs (DMPs) selected based on specificity of methylation status in said DMPs in at least one known sample type among at least one from another known sample types, wherein step B1) preferably comprises:
i) providing at least two parametrized profiles based on pairs of adjacent CpG sites for at least two different known types of cells or tissues generated with the method according to claim 84, wherein the number and the known types of cells or tissues, for which the parametrized profiles are provided, giving a specific application context.
ii) determining and comparing the methylation status contained in the model parameters for each pair of adjacent CpG sites present in all of said at least two parametrized profiles
iii) extracting each pair to be a part of said discriminative map of cells or tissues types under construction if the methylation status in at least one parametrized profile for said pair is different from the methylation status in at least one among all other parametrized profiles.
iiii) saving genomic coordinates for extracted pairs of adjacent CpG sites so as to generate said discriminative map of cells or tissues types for a specific application context, said extracted pairs being differential methylation pairs (DMPs) selected based on specificity of methylation status in said DMPs in at least one known sample type among at least one from another known sample types;
C1) saving, only for pairs of adjacent CpG sites contained in said discriminative map of cell or tissues types,
genomic coordinates of adjacent CpG sites and
compressed methylation information in the form of one single numerical value relating to said adjacent CpG sites,
so as to generate said cell or tissue type discriminative profile based on pairs of adjacent CpG sites for said sample relating to a certain type of tissue or cell within said specific application context;
wherein preferably the methylation status is one among at least comethylation status and non co-methylation status in a pair of adjacent CpG sites and wherein converting two values of methylation level associated with two CpG sites in each pair, into one single numerical value involves calculating methylation value difference between two CpG sites in each pair of said adjacent CpG sites.

88. The method according to claim 87, wherein the Bi) step of b) comprises the step v) of constructing parametrized profile based on pairs of adjacent CpG sites of a known type of tissue or cell based on methylation information derived from nucleic acid contained in a group of at least one sample, each sample relating to the same known type of said tissue or said cell, comprises:
a) for each sample in said group of at least one sample providing digital data on methylation level measurements for CpG sites contained in said nucleic acid in said sample relating to said tissue or said cell of a known type
b) determining a set of pairs of adjacent CpG sites in said nucleic acid contained in each sample in said group of at least one sample relating to said tissue or said cell of a known type, each pair of adjacent CpG sites consisting of two adjacent CpG sites localized within one nucleic acid molecule so as to generate a map of adjacent CpG sites containing genomic coordinates; then
c) for said group of at least one sample iteratively for each pair of adjacent CpG sites among the determined set of pairs of adjacent CpG sites, compressing information on methylation level value, by fitting a parametrized mathematical model to said digital data on values of methylation level measurements associated with each pair of adjacent CpG sites so as to determine one single model parameter, said single model parameter representing compressed methylation information for a pair of adjacent CpG sites and being indicative of methylation status in each pair of adjacent CpG sites for said group of at least one sample; wherein said fitting a parametrized mathematical model to said digital data preferably comprises: for each pair of adjacent CpG sites separately, estimating a regression model such that:
$$MetLevel ∼ intercept + {β}_{i} * POS$$
$$POS = \{ \begin{matrix}0 & if CpG = first \\ 1 & if CpG = successive\end{matrix}$$
wherein intercept and pi are model parameters, the parameter POS being an exogenous variable taking values of 0 if the CpG site in a pair is a first one, or 1 if the CpG site in a pair is a successive one;
d) for said group of at least one sample saving
genomic coordinates of adjacent CpG sites for each pair in said determined set of pairs and
compressed methylation information in the form of one model parameter for said each pair of adjacent CpG sites in said group of at least one sample
so as to generate said parametrized profile based on pairs of adjacent CpG sites of a known type of tissue or cell.

89. The method according to claim 87, wherein said sample characterizing tool is a software module comprising a model for cell or tissue type classifying being provided by the following steps:
a) providing a training data set by
providing a first set of cell or tissue type discriminative profiles based on pairs of adjacent CpG sites within a specific application context for the first known type of the tissue or cell along with associated labels indicating the first known type of tissue or cell
providing at least a second set of cell or tissue type discriminative profile based on pairs of adjacent CpG sites within a specific application context for at least the second known type of the tissue or cell along with associated labels indicating the at least second known type of tissue or cell;
b) training the classification model with said training data set so as it is configured to classify the type of a cell or tissue relating to an input cell or tissue type discriminative profile based on pairs of adjacent CpG sites, said tool being configured to output a result of the classification that indicates the tissue or cell of origin contained in said test sample; or
wherein said sample characterizing tool is a software module for determining a tissue or cell of origin contained in a test sample being provided by the following steps:
a) providing a library of reference discriminative profiles of cell or tissue types based on pairs of adjacent CpG sites, by:
providing a first reference discriminative profile of cell or tissue types based on pairs of adjacent CpG sites within a specific application context for a first known type of cell or tissue;
providing at least a second reference discriminative profile of cell or tissue types based on pairs of adjacent CpG sites within a specific application context for at least a second known type of cell or tissue;
storing said at least two reference discriminative profiles of cell or tissue types based on pairs of adjacent CpG sites so as to generate said library of reference discriminative profiles
b) providing a querying tool; said tool being configured to output a result of the query that indicates the tissue or cell of origin contained in said test sample;
wherein the reference discriminative profile of a known cell or tissue type based on pairs of adjacent CpG sites within a specific application context is preferably generated by:
providing a set of discriminative profiles based on pairs of adjacent CpG sites within said specific application context obtained for a group of at least one sample of the same known type of the tissue or cell;
for each pair of adjacent CpG sites in said discriminative profiles, for all samples in said group of at least one sample of the same cell or tissue type, calculating one among a mean or median value resulting from a set of compressed methylation information in the form of one single numerical value relating to said each pair for said all samples in said group of at least one samples;
saving
(i) genomic coordinates of each pair of adjacent CpG sites and
(ii) said calculated mean value or median of the compressed methylation information in the form of one single numerical value for said each pair so as to generate a reference discriminative profile of a known cell or tissue type based on pairs of adjacent CpG sites within a specific application context;
or wherein said sample characterizing tool is a software module for predicting a sample composition of the test sample, said tool being provided by the following steps:
providing a library of reference discriminative profiles of cell or tissue types based on pairs of adjacent CpG sites;
providing a deconvolution tool;
said tool being configured to output a result of the deconvolution that indicates the composition of said test sample.

90. The method according to claim 88, wherein the model is a reduced trained model for cell or tissue type classifying provided by further eliminating unnecessary pairs of adjacent CpG sites in the training data set by further training said classification model with the use of a feature selection method, then saving reduced discriminative map and saving said reduced trained model.

91. A computing platform for characterizing a test sample from a subject, comprising:
a computing device comprising a processor, a memory module, an operating system, and a computer program including instructions executable by the processor to create a sample characterizing application, the sample characterizing application comprising a data analysis module configured to:
a) receive methylation level data provided by measuring methylation level of consecutive nucleic acid sequence contained in said test sample from the subject;
b) provide for said test sample a CpG pair-based discriminative methylation profile based on methylation level data, wherein the CpG pair-based methylation discriminative profile comprises a differential methylation pairs (DMPs) selected based on specificity of methylation status in said DMPs in at least one sample type among at least one from another sample types, said profile containing genomic coordinates of said consecutive DMPs and a single value indicative of methylation status for said DMPs in said sample;
wherein said data analysis module further comprises a sample characterizing tool configured to determine sample type, said tool outputting the result of sample characterization and using for characterization one or more pre-established methylation signatures, wherein each of the one or more pre-established methylation signatures correlates with sample type, and wherein each pre-established methylation signature comprises a set of differential methylation pairs DMP selected based on specificity of methylation status in said DMPs in at least one sample type among at least one from another sample types.

92. The computing platform according to claim 91, wherein said sample characterizing tool is one among: a machine learning model for cell or tissue type classifying, a library of reference discriminative profiles based on DMPs along with a querying tool for typing cell or tissue contained in said test sample, a library of reference discriminative profiles based on DMPs along with a deconvolution tool for indicating composition of the test sample.
